(19) United States
(12) Patent Application Publication
(10) Pub. No.: US 2025/0388632 A1
(43) Pub. Date: Dec. 25, 2025

(54) ANNEXIN A1-DERIVED POLYPEPTIDE ANALOGUES

(71) Applicant: RESOTHER PHARMA A/S, Holte (DK)

(72) Inventor: Thomas Engelbrecht Nordkild JONASSEN, Holte (DK)

(21) Appl. No.: 18/839,672

(22) PCT Filed: Feb. 23, 2023
(86) PCT No.: PCT/EP2023/054519

§ 371 Date: Aug. 19, 2024

(30) Foreign Application Priority Data
Feb. 23, 2022 (EP) 22158253.9

(51) Int. Cl. C07K 14/47; A61K 38/00

(52) U.S. Cl. CPC C07K 14/47; A61K 38/00

(57) ABSTRACT

The present disclosure relates to polypeptides and polypeptide analogues derived from Annexin A1 as well as compositions comprising said polypeptides or polypeptide analogues for treatment of inflammatory and ischemic conditions.

### TECHNICAL FIELD

The present disclosure relates to polypeptides and polypeptide analogues derived from Annexin A1 as well as compositions comprising said polypeptides or polypeptide analogues for treatment of inflammatory and ischemic conditions.

### SEQUENCE LISTING

The present application includes a sequence listing, which has been submitted in XML format, hereby incorporated by reference, entitled THER0003PASEQ.XML, having a size of 136 kilobytes with a creation date of Mar. 31, 2025.

### BACKGROUND

The Annexin super-family consists of 13 calcium phospholipid binding proteins with significant biological and structural homology. Annexins are structurally divided into a highly conserved core domain and a variable N-terminal domain. Annexin A1 (ANXA1, 37 kDa-SEQ ID NO: 1) is an anti-inflammatory protein that inhibits extravasation of blood-borne polymorphonuclear leukocyte (PMN) into the surrounding tissue. The protein binds to the N-formyl peptide receptor (FPR) 2 or FPR-L1 receptor, where it initiates a cascade of signaling events. Following an inflammatory stimulus, migration of blood-borne polymorphonuclear leukocyte (PMN) into the surrounding tissue takes place. Transmigration or extravasation of PMN is regulated by mediators such as adhesion molecules, cytokines and proteases, which control the pro-inflammatory and anti-inflammatory processes. The disruptive potential of the PMN is high and potentially self-damaging. Thus, controlling extravasation of PMN and the inflammatory response is important.

For therapeutic purposes as an anti-inflammatory agent, the full Annexin A1 protein has numerous disadvantages relative to functional fragments or modified versions thereof. The large size of the protein makes it more difficult to deliver by techniques that are possible with a smaller polypeptide (e.g. transdermally or transmucosally). For use to treat inflammation of the eyes, a smaller molecule is expected to be better able to penetrate the corneal epithelium. Also, susceptibility to proteolytic degradation is a particular concern for all peptide pharmaceuticals, especially large ones and especially if oral delivery (preferred by many patients) is contemplated.

Some Annexin A1 derivatives lacking significant regions on the N-terminal side of the polypeptide have been shown to lack significant activity in some assays of inflammation and mediator release, whereas the full length N-terminus N-acetyl Annexin A1 (2-26) was deemed biologically active in several systems. A number of peptides primarily derived from the unique N-terminal portion of the Annexin A1 protein have been shown to possess anti-inflammatory properties. One of the most extensively studied Annexin A1 peptides is peptide Ac2-26, which mimics the 2nd to the 26th amino acids of the 54-amino acid N-terminal region. Like the Ac 1-188 fragment (and the native protein), it has an N-terminal acetylation to increase its stability, and possibly its half-life. It has been show that Annexin A1 and its N-terminal peptide (Ac2-26) exert the majority of their anti-inflammatory action through the FPR2/Lipoxin A4 (FPR2/AIx) receptor. In vivo the Ac2-26 peptide has been shown to exert an anti-inflammatory effect in models of myocardial ischaemia reperfusion (I/R), mesentery I/R, glycogen peritonitis and IL1 airpouch, where it was reported to significantly reduce the recruitment of neutrophils to the site of injury/inflammation. The anti-inflammatory properties of this peptide are not restricted to acute models of inflammation. In an arthritis model, intra-articular administration of the Ac2-26 peptide was shown to reduce disease severity through a reduction in neutrophil recruitment.

Shorter versions of the Ac2-26 peptide, such as peptides Ac2-12 and Ac2-6, have also been shown to elicit some degree of anti-inflammatory effects in acute models of inflammation. Longer polypeptides with anti-inflammatory effects, such as polypeptides corresponding to amino acid residues 2-48 and 11-48, have been disclosed (WO 2012/174397). Work conducted by a number of laboratories has shown that a peptide derived from a region completely independent of the N-terminal of the Annexin A1 protein, more precisely amino acids 247-253—in the third repeat of the core region of the protein-referred to as antiflammin-2 (AF2), also possesses anti-inflammatory properties.

### SUMMARY

The present disclosure relates to Annexin A1 polypeptide analogues, conjugates comprising Annexin A1 polypeptides and branched amino acid probes (BAPs), as well as compositions comprising Annexin A1 polypeptide analogues or their conjugates for treatment of ischemic and/or inflammatory conditions.

One aspect of the present disclosure relates to a polypeptide or polypeptide analogue comprising at least the sequence AMVSEFLKQAWFIENEEQEYVQTVKS (SEQ ID NO: 2), or a functional variant thereof, wherein said polypeptide consists of a polypeptide selected from the group consisting of SEQ ID NO:2 to SEQ ID NO:24, or a functional variant thereof.

Another aspect of the present disclosure relates to a polypeptide conjugate comprising said polypeptide or polypeptide analogue and one or more branched amino acid probes.

Another aspect of the present disclosure relates to a pharmaceutical composition comprising said polypeptide or polypeptide analogues.

A further aspect of the present disclosure relates to a polypeptide or polypeptide analogue for use in the treatment of an ischemic condition and/or an inflammatory condition.

### DESCRIPTION OF DRAWINGS

FIG. 1: In vitro phagocytosis by monocytes isolated from human blood. Results are depicted as mean±SEM from four experiments, each using monocytes from different healthy volunteers. The data show that Compound 1 (AnxA1 2-29 (V24L)), Compound 2 (AnxA1 2-34 (V24L)) and Compound 3 (AnxA1 2-39 (V24L)) induce phagocytosis to a higher degree than AnxA1 2-50 (2.5 to 3-fold increase compared to vehicle). For details see Example 1.

FIG. 2: In vitro chemotaxis response by polymorphonuclear leukocytes (PMNs). The data show that Compounds 1, 2 and 3 increase PMN chemotaxis compared to vehicle (1.6 to 3-fold increase). For details see Example 2.

### DETAILED DESCRIPTION

The present disclosure relates to polypeptide analogues of Annexin A1, conjugated forms of said polypeptide analogues, as well as compositions comprising polypeptide analogues of Annexin A1 or their conjugated forms. The disclosed polypeptide analogues, conjugates and compositions are particularly effective for use in the treatment of ischemic and inflammatory conditions.

Full length Annexin A1 (Homo sapiens) has the following sequence: >sp|P04083|ANXA1_HUMAN Annexin A1 OS═Homo sapiens GN=ANXA1 PE=1 SV=2

(SEQ ID NO: 1)
MAMVSEFLKQAWFIENEEQEYVQTVKSSKGGPGSAVSPYPTFNPSSDVAA
LHKAIMVKGVDEATIIDILTKRNNAQRQQIKAAYLQETGKPLDETLKKAL
TGHLEEVVLALLKTPAQFDADELRAAMKGLGTDEDTLIEILASRTNKEIR
DINRVYREELKRDLAKDITSDTSGDFRNALLSLAKGDRSEDFGVNEDLAD
SDARALYEAGERRKGTDVNVFNTILTTRSYPQLRRVFQKYTKYSKHDMNK
VLDLELKGDIEKCLTAIVKCATSKPAFFAEKLHQAMKGVGTRHKALIRIM
VSRSEIDMNDIKAFYQKMYGISLCQAILDETKGDYEKILVALCGGN.

It is a first aspect to provide a polypeptide or polypeptide analogue comprising at least the sequence AMVSEFLKQAWFIENEEQEYVQTVKS (SEQ ID NO: 2), or a functional variant thereof, wherein said polypeptide consists of a polypeptide selected from the group consisting of:

(SEQ ID NO: 2)
AMVSEFLKQAWFIENEEQEYVQTVKS
(SEQ ID NO: 3)
AMVSEFLKQAWFIENEEQEYVQTVKSS
(SEQ ID NO: 4)
AMVSEFLKQAWFIENEEQEYVQTVKSSK
(SEQ ID NO: 5)
AMVSEFLKQAWFIENEEQEYVQTVKSSKG
(SEQ ID NO: 6)
AMVSEFLKQAWFIENEEQEYVQTVKSSKGG
(SEQ ID NO: 7)
AMVSEFLKQAWFIENEEQEYVQTVKSSKGGP
(SEQ ID NO: 8)
AMVSEFLKQAWFIENEEQEYVQTVKSSKGGPG
(SEQ ID NO: 9)
AMVSEFLKQAWFIENEEQEYVQTVKSSKGGPGS
(SEQ ID NO: 10)
AMVSEFLKQAWFIENEEQEYVQTVKSSKGGPGSA
(SEQ ID NO: 11)
AMVSEFLKQAWFIENEEQEYVQTVKSSKGGPGSAV
(SEQ ID NO: 12)
AMVSEFLKQAWFIENEEQEYVQTVKSSKGGPGSAVS
(SEQ ID NO: 13)
AMVSEFLKQAWFIENEEQEYVQTVKSSKGGPGSAVSP
(SEQ ID NO: 14)
AMVSEFLKQAWFIENEEQEYVQTVKSSKGGPGSAVSPY
(SEQ ID NO: 15)
AMVSEFLKQAWFIENEEQEYVQTVKSSKGGPGSAVSPYP
(SEQ ID NO: 16)
AMVSEFLKQAWFIENEEQEYVQTVKSSKGGPGSAVSPYPT
(SEQ ID NO: 17)
AMVSEFLKQAWFIENEEQEYVQTVKSSKGGPGSAVSPYPTF
(SEQ ID NO: 18)
AMVSEFLKQAWFIENEEQEYVQTVKSSKGGPGSAVSPYPTFN
(SEQ ID NO: 19)
AMVSEFLKQAWFIENEEQEYVQTVKSSKGGPGSAVSPYPTFNP
(SEQ ID NO: 20)
AMVSEFLKQAWFIENEEQEYVQTVKSSKGGPGSAVSPYPTFNPS
(SEQ ID NO: 21)
AMVSEFLKQAWFIENEEQEYVQTVKSSKGGPGSAVSPYPTFNPSSD
(SEQ ID NO: 22)
AMVSEFLKQAWFIENEEQEYVQTVKSSKGGPGSAVSPYPTFNPSSDVAAL
H
(SEQ ID NO: 23)
AMVSEFLKQAWFIENEEQEYVQTVKSSKGGPGSAVSPYPTFNPSSDVAAL
HK
(SEQ ID NO: 24)
AMVSEFLKQAWFIENEEQEYVQTVKSSKGGPGSAVSPYPTFNPSSDVAAL
HKA,

or a functional variant thereof.

Also provided herewith is a polypeptide selected from the group consisting of SEQ ID NO: 2 to SEQ ID NO:24, or a functional variant thereof.

In preferred embodiments a functional variant does not encompass also functional fragments of the disclosed polypeptides or polypeptide analogues. Preferably the present polypeptides or polypeptide analogues have the length specified by the SEQ ID NO (i.e. number of consecutive amino acids).

Also disclosed herein are functional variants of a polypeptide or polypeptide analogue comprising at least the sequence AMVSEFLKQAWFIENEEQEYVQTVKS (SEQ ID NO: 2), or a functional variant thereof, wherein said polypeptide consists of a functional variant of a polypeptide selected from the group consisting of any one of SEQ ID NO:2 to SEQ ID NO:24.

A polypeptide or polypeptide analogue as defined herewith, including functional variants thereof, in one embodiment activates and/or stimulates one or more of Formyl Peptide Receptor 1 (FPR1), Formyl Peptide Receptor 2 (FPR2) and Formyl Peptide Receptor 3 (FPR3).

A functional polypeptide as defined herewith is in one embodiment a ligand and/or agonist of one or more of Formyl Peptide Receptor 1 (FPR1), Formyl Peptide Receptor 2 (FPR2) and Formyl Peptide Receptor 3 (FPR3).

The term “agonist” in the present context refers to a polypeptide as defined herein, capable of binding to, or in some embodiments, capable of binding to at least some extent and/or activating a receptor, or in some embodiments, activating a receptor to at least some extent. For example, a FPR2 agonist is thus capable of binding to and/or activating the FPR2.

An agonist may be an agonist of several different types of receptors, and thus capable of binding and/or activating several different types of receptors. Said agonist can also be a selective agonist which only binds and activates one type of receptor. The term “antagonist” in the present context refers to a substance capable of inhibiting the effect of a receptor agonist.

Full agonists bind (have affinity for) and activate a receptor, displaying full efficacy at that receptor. “Partial agonists” in the present context are peptides able to bind and activate a given receptor, but having only partial efficacy at the receptor relative to a full agonist. Partial agonists can act as antagonists when competing with a full agonist for receptor occupancy and producing a net decrease in the receptor activation compared to the effects or activation observed with the full agonist alone.

“Selective agonists” in the present context are compounds which are selective and therefore predominantly bind and activates one type of receptor. Thus a selective FPR2 agonist is selective for the FPR2.

Polypeptides or polypeptide analogues according to the present disclosure are in one embodiment capable of binding and activating to some extent one or several formyl peptide receptors and can have different binding affinities and/or different receptor activation efficacy for different receptors. Affinity refers to the number and size of intermolecular forces between a peptide ligand and its receptor, and residence time of the ligand at its receptor binding site; and receptor activation efficacy refers to the ability of the peptide ligand to produce a biological response upon binding to the target receptor and the quantitative magnitude of this response. In some embodiments, such differences in affinity and receptor activation efficacy are determined by receptor binding/activation studies which are conventional in the art, for instance by generating EC₅₀ and Emax values for stimulation of ligand binding in cells expressing one or several types of receptors as mentioned herein, or on tissues expressing the different types of receptors. High affinity means that a lower concentration of a ligand is needed to obtain a binding of 50% of the receptors compared to ligand peptides which have lower affinity; high receptor activation efficacy means that a lower concentration of the peptide is needed to obtain a 50% receptor activation response (low EC₅₀ value), compared to peptides which have lower affinity and/or receptor activity efficacy (higher EC₅₀ value).

In one embodiment, the polypeptides can have differing affinities and/or receptor activation efficacies for two or more of the receptors selected from FPR1, FPR2 and FPR3.

The receptor activation potency of polypeptide agonists can also be measured in p (A50) values which is a conventional method for determining the receptor activation efficacy of an agonist.

In a particular embodiment, a functional polypeptide has binding affinity and/or receptor efficacy for the Formyl Peptide Receptor 2 (FPR2). This may be tested using conventional methods, or as outlined in the examples.

In one particular embodiment, the polypeptide is capable of binding to and activating FPR2. In a further embodiment said peptide is a full agonist of FPR2.

In one embodiment a a polypeptide or polypeptide analogue comprising at least the sequence AMVSEFLKQAWFIENEEQEYVQTVKS (SEQ ID NO: 2), or a functional variant thereof, wherein said polypeptide consists of a polypeptide selected from the group consisting of any one of SEQ ID NO:2 to SEQ ID NO:24, or a functional variant thereof, is capable of one or more of
a) binding to one or more of the formyl peptide receptors, including FPR1, FPR2 and FPR3, and/or
b) activating and/or stimulating one or more of the formyl peptide receptors, including FPR1, FPR2 and FPR3, and/or
c) binding and/or activating FPR2, and/or
d) activating immune cells, and/or
e) activating leukocytes, such as phagocytic leukocytes such as monocytes
f) activating leukocytes, such as polymorphonuclear leukocytes (PMNs), such as neutrophils, and/or
g) activating neutrophils and/or monocytes, and/or
h) activating leukocytes' effector functions, such as one or more of inducing neutrophil chemotaxis, mobilization of neutrophil complement receptor 3 (CR3), and activation of the neutrophil NADPH-oxidase, and/or
i) inducing phagocytosis in leukocytes, such as in phagocytic leukocytes, such as in monocytes, and/or
j) inducing chemotaxis in leukocytes, such as polymorphonuclear leukocytes (PMNs), such as in neutrophils.

A variant of a polypeptide as defined herewith can in principle have one or more substitutions at one or more positions. Individual amino acid residues in the disclosed sequences can be substituted with any given proteinogenic or non-proteinogenic amino acid.

In one embodiment of the present disclosure a functional variant of a polypeptide consisting of a polypeptide selected from the group consisting of any one of SEQ ID NO: 2 to SEQ ID NO:24, has at least 75% sequence identity to any one of SEQ ID NO:2 to SEQ ID NO:24, such as at least 80% sequence identity, such as at least 85% sequence identity, such as at least 90% sequence identity, such as at least 95% sequence identity to any one of SEQ ID NO:2 to SEQ ID NO:24.

In one embodiment of the present disclosure a functional variant of a polypeptide as defined herein is a polypeptide selected from the group consisting of any one of SEQ ID NO: 2 to SEQ ID NO:24 having one or more amino acid substitutions. One amino acid substitution means that the amino acid differs between the original sequence and the variant sequence at one position.

In one embodiment of the present disclosure a functional variant of a polypeptide as defined herein is a polypeptide selected from the group consisting of any one of SEQ ID NO: 2 to SEQ ID NO:24 having one amino acid substitution.

In one embodiment of the present disclosure a functional variant of a polypeptide as defined herein is a polypeptide selected from the group consisting of any one of SEQ ID NO: 2 to SEQ ID NO:24 having two amino acid substitutions.

In one embodiment of the present disclosure a functional variant of a polypeptide as defined herein is a polypeptide selected from the group consisting of any one of SEQ ID NO: 2 to SEQ ID NO:24 having three amino acid substitutions.

In one embodiment of the present disclosure a functional variant of a polypeptide as defined herein is a polypeptide selected from the group consisting of any one of SEQ ID NO: 2 to SEQ ID NO:24 having four amino acid substitutions.

In one embodiment of the present disclosure a functional variant of a polypeptide as defined herein is a polypeptide selected from the group consisting of any one of SEQ ID NO: 2 to SEQ ID NO:24 having five amino acid substitutions.

In one embodiment said one or more amino acid substitutions are conservative amino acid substitutions. In one embodiment said one or more amino acid substitutions are non-conservative amino acid substitutions.

The genetic code specifies 20 standard amino acids naturally incorporated into polypeptides (proteinogenic): Ala, Arg, Asn, Asp, Cys, Gln, Glu, Gly, His, 11e, Leu, Lys, Met, Phe, Pro, Ser, Tyr, Thr, Trp, Val, and 2 which are incorporated into proteins by unique synthetic mechanisms: Sec (selenocysteine, or U) and Pyl (pyrrolysine, O). These are all L-stereoisomers.

Aside from the 22 standard or natural amino acids, there are many other non-naturally occurring amino acids (non-proteinogenic or non-standard). They are either not found in proteins, or are not produced directly and in isolation by standard cellular machinery.

Non-standard amino acids are usually formed through modifications to standard amino acids, such as post-translational modifications. Examples of unnatural amino acid residues are Abu, Aib, Nle (Norleucine), DOrn (D-ornithine, deguanylated arginine), Nal (beta-2-naphthyl-alanine), D-Nal (beta-2-naphthyl-D-alanine), DArg, DTrp, DPhe and DVal.

Any amino acids according to the present disclosure may be in the L- or D-configuration. If nothing is specified, reference to the L-isomeric form is preferably meant.

The term polypeptide also embraces post-translational modifications introduced by chemical or enzyme-catalyzed reactions, as are known in the art. Such post-translational modifications can be introduced prior to partitioning, if desired. Also, functional equivalents may comprise chemical modifications such as ubiquitination, labeling (e.g., with radionuclides, various enzymes, etc.), pegylation (derivatization with polyethylene glycol), or by insertion (or substitution by chemical synthesis) of amino acids, which do not normally occur in human proteins (e.g. ornithine).

Polypeptides as defined herein with N-terminal alkylations and C-terminal esterifications are also encompassed within the present disclosure. Functional equivalents also comprise glycosylated and covalent or aggregative conjugates formed with the same molecules, including dimers or unrelated chemical moieties. Such functional equivalents are prepared by linkage of functionalities to groups which are found in a fragment including at any one or both of the N- and C-termini, by means known in the art.

In one embodiment the alanine residue at position 10 of any one of SEQ ID NO: 2 to SEQ ID NO: 24 is substituted with any other standard or non-standard amino acid.

In one embodiment the alanine residue at position 10 of any one of SEQ ID NO: 2 to SEQ ID NO: 24 is substituted with an amino acid residue independently selected from the group consisting of leucine, aspartic acid, methionine, glutamic acid, isoleucine and arginine.

In one embodiment the residue at position 10 of any one of SEQ ID NO: 2 to SEQ ID NO: 24 is not alanine; i.e. is any amino acid except for alanine.

In one embodiment the valine residue at position 21 of any one of SEQ ID NO: 2 to SEQ ID NO: 24 is substituted with any other standard or non-standard amino acid.

In one embodiment the valine residue at position 21 of any one of SEQ ID NO: 2 to SEQ ID NO: 24 is substituted with an amino acid residue independently selected from the group consisting of leucine, aspartic acid, methionine, glutamic acid, isoleucine and lysine.

In one embodiment the residue at position 21 of any one of SEQ ID NO: 2 to SEQ ID NO: 24 is not valine, i.e. is any amino acid except for valine.

In one embodiment the alanine residue at position 10 and the valine residue at position 21 of any one of SEQ ID NO: 2 to SEQ ID NO: 24 are both substituted with an amino acid residue independently selected from the group consisting of leucine, aspartic acid, methionine, glutamic acid, isoleucine, arginine and lysine; such as the group consisting of leucine, aspartic acid and methionine; such as leucine. In one embodiment residue and residue 21 of any one of SEQ ID NO: 2 to SEQ ID NO: 24 are identical.

In one embodiment the valine residue at position 24 of any one of SEQ ID NO: 2 to SEQ ID NO: 24 is substituted with any other standard or non-standard amino acid.

In one embodiment the residue at position 24 of any one of SEQ ID NO: 2 to SEQ ID NO: 24 is not valine, i.e. is any amino acid except for valine.

In one embodiment the valine residue at position 24 of any one of SEQ ID NO: 2 to SEQ ID NO: 24 is substituted with an amino acid residue independently selected from the group consisting of glycine, alanine, serine, threonine, cysteine, leucine, isoleucine, methionine, proline, phenylalanine, tyrosine, tryptophan, aspartic acid, glutamic acid, asparagine, glutamine, histidine, lysine and arginine.

In a preferred embodiment the valine residue at position 24 of any one of SEQ ID NO: 2 to SEQ ID NO: 24 is substituted with leucine. In some embodiments the Val24Leu mutation increases protease stability.

In one embodiment the valine residue at position 35 of any one of SEQ ID NO: 2 to SEQ ID NO: 24 is substituted with any other standard or non-standard amino acid.

In one embodiment the valine residue at position 35 of any one of SEQ ID NO: 2 to SEQ ID NO: 24 is substituted with an amino acid residue independently selected from the group consisting of glycine, alanine, serine, threonine, cysteine, leucine, isoleucine, methionine, proline, phenylalanine, tyrosine, tryptophan, aspartic acid, glutamic acid, asparagine, glutamine, histidine, lysine and arginine.

In one embodiment the residue at position 35 of any one of SEQ ID NO: 2 to SEQ ID NO: 24 is lysine.

In one embodiment the residue at position 35 of any one of SEQ ID NO: 2 to SEQ ID NO: 24 is not valine, i.e. is any amino acid except for valine.

In one embodiment the leucine residue at position 50 of any one of SEQ ID NO: 2 to SEQ ID NO: 24 is substituted with any other standard or non-standard amino acid.

In one embodiment the leucine residue at position 50 of any one of SEQ ID NO: 23 to SEQ ID NO: 24 is substituted with an amino acid residue independently selected from the group consisting of glycine, alanine, serine, threonine, cysteine, valine, isoleucine, methionine, proline, phenylalanine, tyrosine, tryptophan, aspartic acid, glutamic acid, asparagine, glutamine, histidine, lysine and arginine.

In one embodiment the residue at position 50 of any one of SEQ ID NO: 23 to SEQ ID NO: 24 is any amino acid except for leucine.

In one embodiment there is provided a polypeptide selected from the group consisting of:

(SEQ ID NO: 25)
AMVSEFLKQX₁WFIENEEQEYX₂QTX₃KS,
(SEQ ID NO: 26)
AMVSEFLKQX₁WFIENEEQEYX₂QTX₃KSS,
(SEQ ID NO: 27)
AMVSEFLKQX₁WFIENEEQEYX₂QTX₃KSSK,
(SEQ ID NO: 28)
AMVSEFLKQX₁WFIENEEQEYX₂QTX₃KSSKG,
(SEQ ID NO: 29)
AMVSEFLKQX₁WFIENEEQEYX₂QTX₃KSSKGG,
(SEQ ID NO: 30)
AMVSEFLKQX₁WFIENEEQEYX₂QTX₃KSSKGGP,
(SEQ ID NO: 31)
AMVSEFLKQX₁WFIENEEQEYX₂QTX₃KSSKGGPG,
(SEQ ID NO: 32)
AMVSEFLKQX₁WFIENEEQEYX₂QTX₃KSSKGGPGS,
(SEQ ID NO: 33)
AMVSEFLKQX₁WFIENEEQEYX₂QTX₃KSSKGGPGSA,
(SEQ ID NO: 34)
AMVSEFLKQX₁WFIENEEQEYX₂QTX₃KSSKGGPGSAX₄,
(SEQ ID NO: 35)
AMVSEFLKQX₁WFIENEEQEYX₂QTX₃KSSKGGPGSAX₄S,
(SEQ ID NO: 36)
AMVSEFLKQX₁WFIENEEQEYX₂QTX₃KSSKGGPGSAX₄SP,
(SEQ ID NO: 37)
AMVSEFLKQX₁WFIENEEQEYX₂QTX₃KSSKGGPGSAX₄SPY,
(SEQ ID NO: 38)
AMVSEFLKQX₁WFIENEEQEYX₂QTX₃KSSKGGPGSAX₄SPYP,
(SEQ ID NO: 39)
AMVSEFLKQX₁WFIENEEQEYX₂QTX₃KSSKGGPGSAX₄SPYPT,
(SEQ ID NO: 40)
AMVSEFLKQX₁WFIENEEQEYX₂QTX₃KSSKGGPGSAX₄SPYPTF,
(SEQ ID NO: 41)
AMVSEFLKQX₁WFIENEEQEYX₂QTX₃KSSKGGPGSAX₄SPYPTFN,
(SEQ ID NO: 42)
AMVSEFLKQX₁WFIENEEQEYX₂QTX₃KSSKGGPGSAX₄SPYPTFNP,
(SEQ ID NO: 43)
AMVSEFLKQX₁WFIENEEQEYX₂QTX₃KSSKGGPGSAX₄SPYPTFNPS,
(SEQ ID NO: 44)
AMVSEFLKQX₁WFIENEEQEYX₂QTX₃KSSKGGPGSAX₄SPYPTFNPSS
D,
(SEQ ID NO: 45)
AMVSEFLKQX₁WFIENEEQEYX₂QTX₃KSSKGGPGSAX₄SPYPTFNPSSD
VAAX₅H,
(SEQ ID NO: 46)
AMVSEFLKQX₁WFIENEEQEYX₂QTX₃KSSKGGPGSAX₄SPYPTFNPSSD
VAAX₅HK,
and
(SEQ ID NO: 47)
AMVSEFLKQX₁WFIENEEQEYX₂QTX₃KSSKGGPGSAX₄SPYPTFNPSSD
VAAX₅HKA,

wherein X₁ is selected from the group consisting of alanine, leucine, aspartic acid, methionine, glutamic acid, isoleucine and arginine,
wherein X₂ is selected from the group consisting of valine, leucine, aspartic acid, methionine, glutamic acid, isoleucine and lysine.
wherein X₃ is selected from the group consisting of valine, glycine, alanine, serine, threonine, cysteine, leucine, isoleucine, methionine, proline, phenylalanine, tyrosine, tryptophan, aspartic acid, glutamic acid, asparagine, glutamine, histidine, lysine and arginine; preferably leucine,
wherein X₄ is selected from the group consisting of valine, glycine, alanine, serine, threonine, cysteine, leucine, isoleucine, methionine, proline, phenylalanine, tyrosine, tryptophan, aspartic acid, glutamic acid, asparagine, glutamine, histidine, lysine and arginine; preferably lysine, and
wherein X₅ is selected from the group consisting of leucine, glycine, alanine, serine, threonine, cysteine, valine, isoleucine, methionine, proline, phenylalanine, tyrosine, tryptophan, aspartic acid, glutamic acid, asparagine, glutamine, histidine, lysine and arginine,
or a functional variant thereof having one or more amino acid substitutions, such as having 1, 2, 3, 4 or 5 amino acid substitutions at any position other than X₁, X₂, X₃, X₄ and X₅.

In another embodiment there is provided a polypeptide selected from the group consisting of:

SEQ ID NO: 48)
AMVSEFLKQAWFIENEEQEYVQTLKS,
(SEQ ID NO: 49)
AMVSEFLKQAWFIENEEQEYVQTLKSS,
(SEQ ID NO: 50)
AMVSEFLKQAWFIENEEQEYVQTLKSSK,
(SEQ ID NO: 51)
AMVSEFLKQAWFIENEEQEYVQTLKSSKG,
(SEQ ID NO: 52)
AMVSEFLKQAWFIENEEQEYVQTLKSSKGG,
(SEQ ID NO: 53)
AMVSEFLKQAWFIENEEQEYVQTLKSSKGGP,
(SEQ ID NO: 54)
AMVSEFLKQAWFIENEEQEYVQTLKSSKGGPG,
(SEQ ID NO: 55)
AMVSEFLKQAWFIENEEQEYVQTLKSSKGGPGS,
(SEQ ID NO: 56)
AMVSEFLKQAWFIENEEQEYVQTLKSSKGGPGSA,
(SEQ ID NO: 57)
AMVSEFLKQAWFIENEEQEYVQTLKSSKGGPGSAV,
(SEQ ID NO: 58)
AMVSEFLKQAWFIENEEQEYVQTLKSSKGGPGSAVS,
(SEQ ID NO: 59)
AMVSEFLKQAWFIENEEQEYVQTLKSSKGGPGSAVSP,
(SEQ ID NO: 60)
AMVSEFLKQAWFIENEEQEYVQTLKSSKGGPGSAVSPY,
(SEQ ID NO: 61)
AMVSEFLKQAWFIENEEQEYVQTLKSSKGGPGSAVSPYP,
(SEQ ID NO: 62)
AMVSEFLKQAWFIENEEQEYVQTLKSSKGGPGSAVSPYPT,
(SEQ ID NO: 63)
AMVSEFLKQAWFIENEEQEYVQTLKSSKGGPGSAVSPYPTF,
(SEQ ID NO: 64)
AMVSEFLKQAWFIENEEQEYVQTLKSSKGGPGSAVSPYPTFN,
(SEQ ID NO: 65)
AMVSEFLKQAWFIENEEQEYVQTLKSSKGGPGSAVSPYPTFNP,
(SEQ ID NO: 66)
AMVSEFLKQAWFIENEEQEYVQTLKSSKGGPGSAVSPYPTFNPS,
(SEQ ID NO: 67)
AMVSEFLKQAWFIENEEQEYVQTLKSSKGGPGSAVSPYPTFNPSSD,
(SEQ ID NO: 68)
AMVSEFLKQAWFIENEEQEYVQTLKSSKGGPGSAVSPYPTFNPSSDVAAL
H,
(SEQ ID NO: 69)
AMVSEFLKQAWFIENEEQEYVQTLKSSKGGPGSAVSPYPTFNPSSDVAAL
HK,
and
(SEQ ID NO: 70)
AMVSEFLKQAWFIENEEQEYVQTLKSSKGGPGSAVSPYPTFNPSSDVAAL
HKA,

or a functional variant thereof.

In one embodiment of the present disclosure a functional variant of a polypeptide consisting of a polypeptide selected from the group consisting of any one of SEQ ID NO: 48 to SEQ ID NO:70, has at least 75% sequence identity to any one of SEQ ID NO: 48 to SEQ ID NO:70, such as at least 80% sequence identity, such as at least 85% sequence identity, such as at least 90% sequence identity, such as at least 95% sequence identity to any one of SEQ ID NO:48 to SEQ ID NO:70.

In one embodiment of the present disclosure a functional variant of a polypeptide as defined herein is a polypeptide selected from the group consisting of any one of SEQ ID NO: 48 to SEQ ID NO:70 having one or more amino acid substitutions. One amino acid substitution means that the amino acid differs between the original sequence and the variant sequence at one position.

In one embodiment of the present disclosure a functional variant of a polypeptide as defined herein is a polypeptide selected from the group consisting of any one of SEQ ID NO: 48 to SEQ ID NO:70 having one amino acid substitution.

In one embodiment of the present disclosure a functional variant of a polypeptide as defined herein is a polypeptide selected from the group consisting of any one of SEQ ID NO: 48 to SEQ ID NO:70 having two amino acid substitutions.

In one embodiment of the present disclosure a functional variant of a polypeptide as defined herein is a polypeptide selected from the group consisting of any one of SEQ ID NO: 48 to SEQ ID NO:70 having three amino acid substitutions.

In one embodiment of the present disclosure a functional variant of a polypeptide as defined herein is a polypeptide selected from the group consisting of any one of SEQ ID NO: 48 to SEQ ID NO:70 having four amino acid substitutions.

In one embodiment of the present disclosure a functional variant of a polypeptide as defined herein is a polypeptide selected from the group consisting of any one of SEQ ID NO: 48 to SEQ ID NO:70 having five amino acid substitutions.

In a particular embodiment there is provided a polypeptide or polypeptide analogue consisting of a peptide selected from the group consisting of:

(SEQ ID NO: 50)
AMVSEFLKQAWFIENEEQEYVQTLKSSK,
(SEQ ID NO: 51)
AMVSEFLKQAWFIENEEQEYVQTLKSSKG,
(SEQ ID NO: 52)
AMVSEFLKQAWFIENEEQEYVQTLKSSKGG,
(SEQ ID NO: 53)
AMVSEFLKQAWFIENEEQEYVQTLKSSKGGP,
(SEQ ID NO: 54)
AMVSEFLKQAWFIENEEQEYVQTLKSSKGGPG,
(SEQ ID NO: 55)
AMVSEFLKQAWFIENEEQEYVQTLKSSKGGPGS,
(SEQ ID NO: 56)
AMVSEFLKQAWFIENEEQEYVQTLKSSKGGPGSA,
(SEQ ID NO: 57)
AMVSEFLKQAWFIENEEQEYVQTLKSSKGGPGSAV,
(SEQ ID NO: 58)
AMVSEFLKQAWFIENEEQEYVQTLKSSKGGPGSAVS,
(SEQ ID NO: 59)
AMVSEFLKQAWFIENEEQEYVQTLKSSKGGPGSAVSP,
and
(SEQ ID NO: 60)
AMVSEFLKQAWFIENEEQEYVQTLKSSKGGPGSAVSPY,

or a functional variant thereof.

In one embodiment of the present disclosure a functional variant of a polypeptide as defined herein is a polypeptide selected from the group consisting of any one of SEQ ID NO: 50 to SEQ ID NO:60 having one or more amino acid substitutions. One amino acid substitution means that the amino acid differs between the original sequence and the variant sequence at one position.

In one embodiment of the present disclosure a functional variant of a polypeptide as defined herein is a polypeptide selected from the group consisting of any one of SEQ ID NO: 50 to SEQ ID NO:60 having one amino acid substitution.

In one embodiment of the present disclosure a functional variant of a polypeptide as defined herein is a polypeptide selected from the group consisting of any one of SEQ ID NO: 50 to SEQ ID NO:60 having two amino acid substitutions.

In one embodiment of the present disclosure a functional variant of a polypeptide as defined herein is a polypeptide selected from the group consisting of any one of SEQ ID NO: 48 to SEQ ID NO:70 having three amino acid substitutions.

In one embodiment of the present disclosure a functional variant of a polypeptide as defined herein is a polypeptide selected from the group consisting of any one of SEQ ID NO: 50 to SEQ ID NO:60 having four amino acid substitutions.

In one embodiment of the present disclosure a functional variant of a polypeptide as defined herein is a polypeptide selected from the group consisting of any one of SEQ ID NO: 50 to SEQ ID NO:60 having five amino acid substitutions.

In a preferred embodiment the polypeptide or polypeptide analogue is selected from the group of:

(SEQ ID NO: 50)
AMVSEFLKQAWFIENEEQEYVQTLKSSK,
(SEQ ID NO: 55)
AMVSEFLKQAWFIENEEQEYVQTLKSSKGGPGS,
and
(SEQ ID NO: 60)
AMVSEFLKQAWFIENEEQEYVQTLKSSKGGPGSAVSPY,

or a functional variant thereof.

In one embodiment of the present disclosure a functional variant of a polypeptide as defined herein is a polypeptide selected from the group consisting of any one of SEQ ID NO: 50, SEQ ID NO:55 and SEQ ID NO:60 having one or more amino acid substitutions. One amino acid substitution means that the amino acid differs between the original sequence and the variant sequence at one position.

In one embodiment of the present disclosure a functional variant of a polypeptide as defined herein is a polypeptide selected from the group consisting of any one of SEQ ID NO: 50, SEQ ID NO:55 and SEQ ID NO:60 having one amino acid substitution.

In one embodiment of the present disclosure a functional variant of a polypeptide as defined herein is a polypeptide selected from the group consisting of any one of SEQ ID NO: 50, SEQ ID NO:55 and SEQ ID NO:60 having two amino acid substitutions.

In one embodiment of the present disclosure a functional variant of a polypeptide as defined herein is a polypeptide selected from the group consisting of any one of SEQ ID NO: 50, SEQ ID NO:55 and SEQ ID NO:60 having three amino acid substitutions.

In one embodiment of the present disclosure a functional variant of a polypeptide as defined herein is a polypeptide selected from the group consisting of any one of SEQ ID NO: 50, SEQ ID NO:55 and SEQ ID NO:60 having four amino acid substitutions.

In one embodiment of the present disclosure a functional variant of a polypeptide as defined herein is a polypeptide selected from the group consisting of any one of SEQ ID NO: 50, SEQ ID NO:55 and SEQ ID NO:60 having five amino acid substitutions.

In one embodiment said one or more amino acid substitutions are conservative amino acid substitutions. In one embodiment said one or more amino acid substitutions are non-conservative amino acid substitutions.

In one embodiment of the present disclosure, the polypeptide or polypeptide analogue including any functional variants as defined herein is acetylated. Any suitable residue in the polypeptide backbone or side chains may be acetylated. In one embodiment, the polypeptide is acetylated at the N-terminus.

In one embodiment of the present disclosure, the polypeptide or polypeptide analogue including any functional variants as defined herein is acetylated (COCH₃ or Ac—) at the N-terminal amino acid residue (e.g. position 1, in one embodiment alanine).

In one embodiment of the present disclosure, the polypeptide or polypeptide analogue including any functional variants as defined herein is amidated (—NH₂) at the C-terminus.

In one embodiment of the present disclosure, the polypeptide, polypeptide analogue or functional variant thereof
a. binds to one or more of the formyl peptide receptors, including Formyl Peptide Receptor 1 (FPR1), Formyl Peptide Receptor 2 (FPR2) and Formyl Peptide Receptor 3 (FPR3), and/or
b. activates and/or stimulates one or more of the formyl peptide receptors, including Formyl Peptide Receptor 1 (FPR1), Formyl Peptide Receptor 2 (FPR2) and Formyl Peptide Receptor 3 (FPR3), and/or
c. is a ligand and/or agonist of one or more of the formyl peptide receptors, including Formyl Peptide Receptor 1 (FPR1), Formyl Peptide Receptor 2 (FPR2) and Formyl Peptide Receptor 3 (FPR3), and/or
d. binds, activates and/or is an agonist for FPR2, and/or
e. activates immune cells, and/or
f. activates leukocytes, such as phagocytic leukocytes, such as monocytes, and/or
g. activates leukocytes, such as polymorphonuclear leukocytes (PMNs), such as neutrophils, and/or
h. activates neutrophils and/or monocytes, and/or
i. activates leukocytes' effector functions, such as one or more of inducing neutrophil chemotaxis, mobilization of neutrophil complement receptor 3 (CR3), and activation of the neutrophil NADPH-oxidase, and/or
j. induces phagocytosis in leukocytes, such as in phagocytic leukocytes, such as in monocytes, and/or
k. induces chemotaxis in leukocytes, such as polymorphonuclear leukocytes (PMNs), such as neutrophils.

### Branched Amino Acid Probes (BAPs)

It is also an aspect of the present disclosure to provide a polypeptide or polypeptide analogue of the present disclosure which is modified by addition of one or more ‘branched amino acid probes’. Branched amino acid probes (BAP) are disclosed in WO 2015/162485. In one embodiment the polypeptide or polypeptide analogue of the present disclosure are conjugates comprising said polypeptide and one or more branched amino acid probes.

In some embodiments, the polypeptide or polypeptide analogue conjugates as provided herein have certain improved properties compared to the corresponding native or unconjugated polypeptide. In one embodiment the BAP conjugates provided herein have increased binding affinity and/or activation of one or more relevant receptors, such as FPRs. In another embodiment, the BAP conjugates provided herein are more stable, such as less susceptible to proteases. Still further, in one embodiment the BAP conjugates have higher solubility.

It is an aspect to provide a polypeptide conjugate comprising a polypeptide or polypeptide analogue comprising at least the sequence AMVSEFLKQAWFIENEEQEYVQTVKS (SEQ ID NO: 2), or a functional variant thereof, wherein said polypeptide consists of a polypeptide selected from the group consisting of SEQ ID NO:2 to SEQ ID NO:24, or a functional variant thereof, and one or more branched amino acid probes consisting of 2 to 9 amino acid residues, wherein the branch point of the branched amino acid probe is formed by an amide bond between the s-amino group of one amino acid residue with the carboxyl group of another amino acid residue, all other bonds being regular peptide bonds, and wherein one or more alpha amino groups of the branched amino acid probe are optionally acetylated.

It is also an aspect to provide a polypeptide conjugate comprising
a polypeptide or polypeptide analogue comprising at least the sequence AMVSEFLKQAWFIENEEQEYVQTVKS (SEQ ID NO: 2), or a functional variant thereof, wherein said polypeptide consists of a polypeptide selected from the group consisting of SEQ ID NO:2 to SEQ ID NO:24, or a functional variant thereof,
and one or more branched amino acid probes,
wherein said branched amino acid probe comprises a first amino alkyl amino acid residue,
said first amino alkyl amino acid residue (AAA) optionally being covalently linked to a second amino alkyl amino acid residue, or to a second and a third amino alkyl amino acid residue, to form a linear chain of 2 or 3 amino alkyl amino acid residues,
wherein the side chain of one or more of said first, second and/or third amino alkyl amino acid residues are each modified by attaching to the side chain amino group a molecule independently selected from the group consisting of: AAAq-AAA; (aa3)p-AAAq; AAAq-(aa3)p; [(aa3)-AAA]p; and [AAA-(aa3)]p;
wherein q is a number selected from 0, 1, 2 and 3; p is a number selected from 1, 2 and 3; AAA is an amino alkyl amino acid residue; and (aa3) is an amino acid residue independently selected from Arg, His, Gly and Ala,
wherein said first amino alkyl amino acid residue is covalently linked to the N-terminus of said polypeptide or polypeptide analogue, covalently linked to the C-terminus of said polypeptide or polypeptide analogue, and/or covalently linked to the side chain amino group of an amino alkyl amino acid residue within said polypeptide or polypeptide analogue,
with the proviso that said branched amino acid probe consists of 2 to 9 amino acid residues.

It is a further aspect to provide a polypeptide conjugate comprising
a polypeptide selected from the group consisting of SEQ ID NO:2 to SEQ ID NO:24, SEQ ID NO:25 to SEQ ID NO:47, and SEQ ID NO:48 to SEQ ID NO:70, or a functional variant thereof, and
one or more branched amino acid probes consisting of 2 to 9 amino acid residues, wherein the branch point of the branched amino acid probe is formed by an amide bond between the s-amino group of one amino acid residue with the carboxyl group of another amino acid residue, all other bonds being regular peptide bonds, and wherein one or more alpha amino groups of the branched amino acid probe are optionally acetylated.

It is also an aspect to provide a polypeptide conjugate comprising
a polypeptide selected from the group consisting of SEQ ID NO:2 to SEQ ID NO:24, SEQ ID NO:25 to SEQ ID NO:47, and SEQ ID NO:48 to SEQ ID NO:70, or a functional variant thereof, and
one or more branched amino acid probes,
wherein said branched amino acid probe comprises a first amino alkyl amino acid residue,
said first amino alkyl amino acid residue (AAA) optionally being covalently linked to a second amino alkyl amino acid residue, or to a second and a third amino alkyl amino acid residue, to form a linear chain of 2 or 3 amino alkyl amino acid residues, wherein the side chain of one or more of said first, second and/or third amino alkyl amino acid residues are each modified by attaching to the side chain amino group a molecule independently selected from the group consisting of: AAAq-AAA; (aa3)p-AAAq; AAAq-(aa3)p; [(aa3)-AAA]p; and [AAA-(aa3)]p;
wherein q is a number selected from 0, 1, 2 and 3; p is a number selected from 1, 2 and 3; AAA is an amino alkyl amino acid residue; and (aa3) is an amino acid residue independently selected from Arg, His, Gly and Ala,
wherein said first amino alkyl amino acid residue is covalently linked to the N-terminus of said polypeptide or polypeptide analogue, covalently linked to the C-terminus of said polypeptide or polypeptide analogue, and/or covalently linked to the side chain amino group of an amino alkyl amino acid residue within said polypeptide or polypeptide analogue,
with the proviso that said branched amino acid probe consists of 2 to 9 amino acid residues.

In one embodiment the N-terminal amino acid residue of the molecule is acetylated at the alpha amino group.

In one embodiment said first amino alkyl amino acid residue is linked by a peptide bond (amide) formed by a reaction of the carboxylic acid, or a derivative thereof, of said first amino alkyl amino acid with the alpha amino group of the N-terminal amino acid residue of said polypeptide; linked by a peptide bond to the C-terminal amino acid residue of said polypeptide formed by reacting the alpha amino group of said amino alkyl amino acid residue with the carboxylic acid, or derivative thereof, of said C-terminal amino acid residue; and/or linked to an amino alkyl amino acid residue within said polypeptide by an amide formed by a reaction of the carboxylic acid, or a derivative thereof, of said first amino alkyl amino acid residue with the alkyl amino group of the amino alkyl amino acid residue.

As defined herewith an amino acid residue being covalently linked to further amino acid residues and/or a peptide in one embodiment means that a peptide bond is present. In another embodiment an amino acid residue being covalently linked to the side chain amino group of an amino alkyl amino acid residue within said polypeptide means that an amide bond is present.

A peptide bond (amide bond) is a covalent chemical bond formed between two molecules when the carboxyl group of one molecule reacts with the amino group of the other molecule, causing the release of a molecule of H₂O. The process usually occurs between amino acids.

If the branched amino acid probe is to be covalently linked to the N-terminus of said polypeptide, the N-terminal amino alkyl amino acid residue of the backbone of the branched amino acid probe is preferably acetylated.

If the branched amino acid probe is to be covalently linked to the side chain amino group of an amino alkyl amino acid residue within said polypeptide, the N-terminal amino alkyl amino acid residue of the backbone of the branched amino acid probe is preferably acetylated.

If the branched amino acid probe is to be covalently linked to the C-terminus of said polypeptide, the C-terminal amino alkyl amino acid residue of the backbone of the branched amino acid probe is preferably a carboxylic acid, an aldehyde, an ester, or an amide, such as a primary amide; most preferably amidated.

The amino alkyl amino acid residues (or AAA) and the amino acid residues (aa₃) according to the disclosure may each be the same (identical) or different (non-identical).

Amino alkyl amino acid residue According to the present disclosure, each branched amino acid probe comprises at least one, such as two, such as three amino alkyl amino acid residue(s).

According to the present disclosure an ‘amino alkyl amino acid residue’ (or AAA) is an amino acid having the conventional amine (—NH₂) and carboxylic acid (—COOH) functional groups, and a side chain covalently linked to the first (alpha-) carbon atom, wherein the side-chain comprises an amino alkyl group (—CₙH₂ₙNH₂).

Thus an amino alkyl amino acid residue (or AAA) is an amino acid with a side chain comprising or consisting of an amino alkyl group (—CₙH₂ₙNH₂), in one embodiment denoted a side chain amino alkyl group.

In one embodiment the side chain alkyl group is derived from the group consisting of methyl (CH₃—), ethyl (C₂H₅—), propyl (C₃H₇—), butyl (C₄H₉—), pentyl (C₅H₁₁—), hexyl (C₆H₁₃), heptyl (C₇H₁₅—), octyl (C₈H₁₇—), nonyl (C₉H₁₉—), decyl (C₁₀H₂₁—), undecyl (C₁₁H₂₃—) and dodecyl (C₁₂H₂₅—). When an alkyl residue having a specific number of carbons is named, all geometric isomers having that number of carbons are intended to be encompassed; thus, for example, “butyl” is meant to include n-butyl, sec-butyl, isobutyl and t-butyl.

In one embodiment the side chain amino group (NH₂) of said amino alkyl amino acid residue is the amine of methylamine, the amine of ethylamine, the amine of propylamine, the amine of n-butylamine, the amine of pentylamine, the amine of n-hexylamine, the amine of heptylamine, the amine of octylamine, the amine of nonylamine, the amine of decylamine, the amine of undecylamine or the amine of dodecylamine.

In one embodiment the side chain amino alkyl group is selected from the group consisting of methylamine (—CH₂NH₂), ethylamine (—C₂H₄NH₂), propylamine (—C₃H₆NH₂), n-butylamine (—C₄H₈NH₂), pentylamine (—C₅H₁₀NH₂), n-hexylamine (—C₆H₁₂NH₂), heptylamine (—C₇H₁₄NH₂), octylamine (—C₈H₁₆NH₂), nonylamine (—C₉H₁₈NH₂), decylamine (—C₁₀H₂₀NH₂), undecylamine (—C₁₁H₂₂NH₂) and dodecylamine (—C₁₂H₂₄NH₂).

In one embodiment of the present disclosure the side chain amino group (NH₂) of said first, second and/or third amino alkyl amino acid residues comprised in each BAP are each modified by attaching a molecule thereto.

In one embodiment the side chain amino group of said amino alkyl amino acid residue comprised in each BAP is selected from the group consisting of
the β (beta) amino group (1 methylene in the side chain; methylamine);
the γ (gamma) amino group (2 methylenes in the side chain, ethylamine);
the δ (delta) amino group (3 methylenes in the side chain, propylamine); =ornithine
the ε (epsilon) amino group (4 methylenes in the side chain; n-butylamine); =lysine
the ζ (zeta) amino group (5 methylenes in the side chain; pentylamine);
the η (eta) amino group (6 methylenes in the side chain; n-hexylamine);
the θ (theta) amino group (7 methylenes in the side chain; heptylamine);
the ι (iota) amino group (8 methylenes in the side chain; octylamine);
the κ (kappa) amino group (9 methylenes in the side chain; nonylamine);
the λ (lambda) amino group (10 methylenes in the side chain; decylamine);
the μ (mu) amino group (11 methylenes in the side chain; undecylamine); and
the v (nu) amino group (12 methylenes in the side chain; dodecylamine).

For example, the s-amino group is covalently linked to the fifth carbon beginning from (including) the α-carbon, which α-carbon is covalently linked to the carboxyl (C═OOH) group.

An amino alkyl amino acid residue wherein the side chain is n-butylamine and the side chain amino group is the ε (epsilon) amino group is lysine (Lys, K).

Likewise, the δ-amino group is covalently linked to the fourth carbon beginning from the α-carbon.

An amino alkyl amino acid residue wherein the side chain is propylamine and the side chain amino group is the δ (delta) amino group is ornithine (Orn).

Ornithine is formed in cells by deguanidation of arginine. While it is not used in proteinogenesis in vivo it is a participant in several enzyme pathways and appears to play a role in nitrogen balance in vivo as it can be gaunidated enzymatically to form arginine.

Any amino acid according to the present disclosure may be in the L- or D-configuration. If nothing is specified, reference to the L-isomeric form is preferably meant.

It follows that the amino alkyl amino acid residues in one embodiment are individually in the L- or D-configuration. In one embodiment the amino alkyl amino acid residues are in the L-configuration.

In one embodiment the amino alkyl amino acid residues comprised in the branched amino acid probe are individually selected from the group consisting of lysine and ornithine.

In one embodiment the amino alkyl amino acid residues are selected from the group consisting of lysine and D-lysine. In a particular embodiment the amino alkyl amino acid residues are lysine residues.

In one embodiment the amino alkyl amino acid residues are selected from the group consisting ornithine and D-ornithine.

In one embodiment of the present disclosure there is provided a polypeptide or polypeptide analogue as defined herewith and one or more branched amino acid probes,
wherein said branched amino acid probe comprises a first amino acid residue selected from lysine and ornithine,
said first amino acid residue optionally being covalently linked to a second, or to a second and a third amino acid residue selected from lysine or ornithine, to form a linear chain of 2 or 3 lysine or ornithine residues,
wherein the side chain of one or more of said first, second and/or third lysine or ornithine residues are modified by attaching to the δ-amino group (ornithine) or the ε-amino group (lysine) a molecule independently selected from the group consisting of

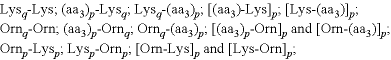

wherein q is a number selected from 0, 1, 2 and 3; p is a number selected from 1, 2 and 3; and (aa₃) is an amino acid residue independently selected from Arg, His, Gly and Ala,
wherein said first lysine or ornithine residue is covalently linked to the N-terminus of said polypeptide or polypeptide analogue, covalently linked to the C-terminus of said polypeptide or polypeptide analogue, and/or covalently linked to the s-amino group of a lysine residue or the δ-amino group of an ornithine residue within said polypeptide or polypeptide analogue,
with the proviso that said branched amino acid probe consists of 2 to 9 amino acid residues.

In one embodiment there is provided a polypeptide or polypeptide analogue as defined herewith and one or more branched amino acid probes,
wherein said branched amino acid probe comprises a first lysine residue, said first lysine residue optionally being covalently linked to a second, or to a second and a third lysine residue, to form a linear chain of 2 or 3 lysine residues,
wherein the side chain of one or more of said first, second and/or third lysine residues are modified by attaching to the s-amino group of said lysine a molecule independently selected from the group consisting of Lys_{q}-Lys; (aa₃)ₚ-Lys_{q}; Lys_{q}-(aa₃)ₚ; [(aa₃)-Lys]ₚ; [Lys-(aa₃)]ₚ; wherein q is a number selected from 0, 1, 2 and 3; p is a number selected from 1, 2 and 3; and (aa₃) is an amino acid residue independently selected from Arg, His, Gly and Ala,
wherein said first lysine residue is covalently linked to the N-terminus of said polypeptide or polypeptide analogue, covalently linked to the C-terminus of said polypeptide or polypeptide analogue, and/or covalently linked to the s-amino group of a lysine or δ-amino group of an ornithine residue within said polypeptide or polypeptide analogue,
with the proviso that said branched amino acid probe consists of 2 to 9 amino acid residues.

### Branching the Probe

A branched amino acid probe (BAP) according to the present disclosure in one embodiment consists of 2 to 9 amino acid residues.

In one embodiment of the present disclosure each of said one or more branched amino acid probe consist of from 2 to 3 amino acid residues, such as from 3 to 4 amino acid residues, for example from 4 to 5 amino acid residues, such as from 5 to 6 amino acid residues, for example from 6 to 7 amino acid residues, such as from 7 to 8 amino acid residues, for example from 8 to 9 amino acid residues.

In one embodiment of the present disclosure each of said one or more branched amino acid probe consist of 2 amino acid residues, such as 3 amino acid residues, for example 4 amino acid residues, such as 5 amino acid residues, for example 6 amino acid residues, such as 7 amino acid residues, for example 8 amino acid residues, such as 9 amino acid residues. In a particular embodiment of the present disclosure each of said one or more branched amino acid probes consists of 3 amino acid residues.

In one embodiment of the present disclosure the branched amino acid probe comprises a first amino alkyl amino acid residue (also denoted AAA₁), which first amino alkyl amino acid residue is connected to the polypeptide or polypeptide analogue as defined herein, to provide a polypeptide conjugate according to the present disclosure.

In one embodiment of the present disclosure the first amino alkyl amino acid of (each of) the one or more branched amino acid probe(s) is covalently linked to the N-terminus of the polypeptide or polypeptide analogue of the present disclosure, covalently linked to the C-terminus of said polypeptide, and/or covalently linked to the side chain amino group of an amino alkyl amino acid residue within said polypeptide.

In one embodiment of the present disclosure the branched amino acid probe comprises a first amino alkyl amino acid residue. In one embodiment the side chain of said first amino alkyl amino acid residue is modified by attaching to the side chain amino group a molecule as defined herein.

In one embodiment of the present disclosure the first amino alkyl amino acid of the branched amino acid probe is acetylated at the alpha amino group. In one embodiment of the present disclosure the N-terminus of the first amino alkyl amino acid residue of the branched amino acid probe is acetylated.

In one embodiment of the present disclosure the N-terminus of the first amino alkyl amino acid residue of the branched amino acid probe is acetylated when the branched amino acid probe comprising said first amino alkyl amino acid residue is covalently linked to the N-terminus of the polypeptide as defined herein; or when the branched amino acid probe comprising said first amino alkyl amino acid residue is covalently linked to the side chain amino group of an amino alkyl amino acid residue within said polypeptide.

In one embodiment of the present disclosure the C-terminus of the first amino alkyl amino acid residue of the branched amino acid probe is a carboxylic acid, an aldehyde, an ester, or an amide, such as a primary amide (CONH₂). In a preferred embodiment of the present disclosure the C-terminus of the first amino alkyl amino acid residue is amidated.

In one embodiment of the present disclosure the C-terminus of the first amino alkyl amino acid residue of the branched amino acid probe is an amide when the branched amino acid probe comprising said first amino alkyl amino acid residue is covalently linked to the C-terminus of the polypeptide as defined herein.

In one embodiment of the present disclosure said first amino alkyl amino acid residue is covalently linked to a second amino alkyl amino acid residue to form a linear chain of 2 amino alkyl amino acid residues. In one embodiment of the present disclosure the alpha-amino group of the second amino alkyl amino acid residue of the branched amino acid probe is acetylated. In one embodiment the N-terminus of the second amino alkyl amino acid residue of the branched amino acid probe is acetylated.

In one embodiment of the present disclosure the C-terminus of the second amino alkyl amino acid residue of the branched amino acid probe is a carboxylic acid, an aldehyde, an ester, or an amide, such as a primary amide (CONH₂). In a preferred embodiment of the present disclosure the C-terminus of the second amino alkyl amino acid residue is amidated.

In one embodiment of the present disclosure said first amino alkyl amino acid residue is covalently linked to a second and (covalently linked to) a third amino alkyl amino acid residue to form a linear chain of 3 amino alkyl amino acid residues.

In one embodiment of the present disclosure the alpha-amino group of the third amino alkyl amino acid residue of the branched amino acid probe is acetylated. In one embodiment of the present disclosure the N-terminus of the third amino alkyl amino acid residue of the branched amino acid probe is acetylated.

In one embodiment of the present disclosure the C-terminal of the third amino alkyl amino acid residue of the branched amino acid probe is a carboxylic acid, an aldehyde, an ester, or an amide, such as a primary amide (CONH₂). In a preferred embodiment of the present disclosure the C-terminus of the third amino alkyl amino acid residue is amidated.

In one embodiment of the present disclosure the first amino alkyl amino acid residue have both the second and third amino alkyl amino acid residues attached at its amine group. In one embodiment of the present disclosure the first amino alkyl amino acid residue have both the second and third amino alkyl amino acid residues covalently linked to its carboxylic acid group. In one embodiment the first amino alkyl amino acid residue has the second amino alkyl amino acid residue attached at its amine group and the third amino alkyl amino acid residue attached at its carboxylic acid group.

The second and third amino alkyl amino acid residues may be denoted AAA₂ and AAA₃, respectively.

In one embodiment of the present disclosure each of said first, second and/or third amino alkyl amino acid residues is an amino acid having a side chain amino alkyl group selected from the group consisting of methylamine (—CH₂NH₂), ethylamine (—C₂H₄NH₂), propylamine (—C₃H₆NH₂), n-butylamine (—C₄H₈NH₂), pentylamine (—C₅H₁₀NH₂), n-hexylamine (—C₆H₁₂NH₂), heptylamine (—C₇H₁₄NH₂), octylamine (—C₈H₁₆NH₂), nonylamine (—C₉H₁₈NH₂), decylamine (—C₁₀H₂₀NH₂), undecylamine (—C₁₁H₂₂NH₂) and dodecylamine (—C₁₂H₂₄NH₂).

In one embodiment of the present disclosure each of the first, second and/or third amino alkyl amino acid residues of the branched amino acid probe are individually selected from the group consisting of lysine, D-lysine, ornithine and D-ornithine.

In one embodiment of the present disclosure each of the first, second and third amino alkyl amino acid residues of the branched amino acid probe are lysine residues (including L-lysine and D-lysine).

In one embodiment of the present disclosure the first, the second or the third amino alkyl amino acid residues of the branched amino acid probe are acetylated at the alpha amino group (Ac-AAA) (COCH₃).

In one embodiment of the present disclosure, the first, the first and second, and the first, second and third amino alkyl amino acid residues of the branched amino acid probe are referred to as the amino alkyl amino acid backbone of the branched amino acid probe (AAA₁, AAA₁-2, AAA₁₋₃).

In one embodiment of the present disclosure the first, the first and second, and the first and second and third amino alkyl amino acid residues are each lysine residues. In one embodiment the first, the first and second, and the first, second and third lysine residues of the branched amino acid probe are referred to as the lysine backbone of the branched amino acid probe (Lys₁, Lys₁₋₂, Lys₁₋₃).

In one embodiment of the present disclosure the first lysine residue, or the second lysine residue, or the third lysine residue of the lysine backbone of the branched amino acid probe is acetylated at the alpha-amino group (Ac-Lys).

In one embodiment of the present disclosure the side chain of one of said first, second and/or third amino alkyl amino acid residues are modified by attaching to the side chain amino group a molecule as defined herein.

In one embodiment of the present disclosure the branched amino acid probe comprises a first amino alkyl amino acid residue, wherein the side chain of said first amino alkyl amino acid residue is modified by attaching to the side chain amino group a molecule as defined herein.

In one embodiment of the present disclosure the branched amino acid probe comprises a first and a second amino alkyl amino acid residue, wherein the side chain of said first amino alkyl amino acid residue is modified by attaching to the side chain amino group a molecule as defined herein.

In one embodiment of the present disclosure the branched amino acid probe comprises a first and a second amino alkyl amino acid residue, wherein the side chain of said second amino alkyl amino acid residue is modified by attaching to the side chain amino group a molecule as defined herein.

In one embodiment of the present disclosure the branched amino acid probe comprises a first and a second amino alkyl amino acid residue, wherein the side chains of said first and second amino alkyl amino acid residue are modified by attaching to the side chain amino group a molecule as defined herein.

In one embodiment of the present disclosure the side chain of two of said first, second and/or third amino alkyl amino acid residues are modified by attaching to the side chain amino group a molecule as defined herein.

In one embodiment of the present disclosure the side chain of all three of the first, second and third amino alkyl amino acid residues are modified by attaching to the side chain amino group a molecule as defined herein.

In one embodiment of the present disclosure the side chain of i) the first amino alkyl amino acid residue, ii) the second amino alkyl amino acid residue, iii) the third amino alkyl amino acid residue, iv) the first and the second amino alkyl amino acid residues, v) the first and the third amino alkyl amino acid residues, vi) the second and the third amino alkyl amino acid residues, or vii) the first, the second and the third amino alkyl amino acid residues, are modified by attaching to the side chain amino group a molecule as defined herein.

In one embodiment of the present disclosure the first lysine residue, or the second lysine residue, or the third lysine residue, or the first and the second lysine residues, or the first and the third lysine residues, or the second and the third lysine residues, or the first, the second and the third lysine residues of the lysine backbone of the branched amino acid are modified by attaching a molecule to the s-amino group.

In one embodiment of the present disclosure the side chain of one or more of each of said first, second and/or third amino alkyl amino acid residues is modified by attaching to the side chain amino group a molecule independently selected from the group consisting of AAA_{q}-AAA; (aa₃)ₚ-AAA_{q}; AAA_{q}-(aa₃)ₚ; [(aa₃)-AAA]ₚ and [AAA-(aa₃)]ₚ; wherein q is a number selected from 0, 1, 2 and 3; p is a number selected from 1, 2 and 3; AAA is an amino alkyl amino acid residue; and (aa₃) is an amino acid residue independently selected from Arg, His, Gly and Ala. In one embodiment the N-terminal AAA or (aa) 3 of the molecule is acetylated at the alpha amino group.

In one embodiment of the present disclosure the side chain of one or more of each of said first, second and/or third amino alkyl amino acid residues is modified by attaching to the side chain amino group a molecule independently selected from the group consisting of

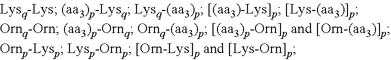

wherein q is a number selected from 0, 1, 2 and 3; p is a number selected from 1, 2 and 3; and (aa₃) is an amino acid residue independently selected from Arg, His, Gly and Ala. In one embodiment the N-terminal Lys, Orn or (aa)₃ of the molecule is acetylated at the alpha amino group.

In one embodiment of the present disclosure the side chain of one or more of each of said first, second and/or third amino alkyl amino acid residues is modified by attaching to the side chain amino group a molecule independently selected from the group consisting of Lys_{q}-Lys; (aa₃)ₚ-LyS_{q}; LyS_{q}-(aa₃)ₚ; [(aa₃)-Lys], and [Lys-(aa₃)]ₚ; wherein q is a number selected from 0, 1, 2 and 3; p is a number selected from 1, 2 and 3; Lys is a lysine residue selected from L-Lys and D-Lys; and (aa₃) is an amino acid residue independently selected from Arg, His, Gly and Ala. In one embodiment the N-terminal Lys or (aa)₃ of the molecule is acetylated at the alpha amino group.

In one embodiment of the present disclosure the side chain of one or more of each of said first, second and/or third lysine residues of the lysine backbone is modified by attaching to the ε-amino group of the side chain a molecule independently selected from the group consisting of Lys_{q}-Lys; (aa₃)ₚ-LyS_{q}; LyS_{q}-(aa₃)ₚ; [(aa₃)-Lys]ₚ and [Lys-(aa₃)]ₚ; wherein q is a number selected from 0, 1, 2 and 3; p is a number selected from 1, 2 and 3; Lys is a lysine residue selected from L-Lys and D-Lys; and (aa₃) is an amino acid residue independently selected from Arg, His, Gly and Ala. In one embodiment the N-terminal Lys or (aa)₃ of the molecule is acetylated at the alpha amino group.

In one embodiment of the present disclosure the side chain of one or more of each of said first, second and/or third lysine residues of the lysine backbone are modified by attaching to the ε-amino group of the side chain a molecule being Lys_{q}-Lys; wherein q is a number selected from 0, 1, 2 and 3. In one embodiment the N-terminal Lys of the molecule is acetylated at the alpha amino group.

In one embodiment of the present disclosure the molecule to be covalently linked to the ε-amino group of the one or more lysine residues of the lysine backbone of the branched amino acid probe are independently selected from the group consisting of Lys_{q}-Lys; (aa₃)ₚ-Lys_{q}; LyS_{q}-(aa₃)ₚ; [(aa₃)-Lys]ₚ and [Lys-(aa₃)]ₚ, wherein q is a number selected from 0, 1, 2 and 3; p is a number selected from 1, 2 and 3, and (aa₃) is an amino acid residue independently selected from Arg, His, Gly and Ala. In one embodiment the N-terminal Lys or (aa)₃ of the molecule is acetylated at the alpha amino group.

It follows that in one embodiment of the present disclosure the first lysine residue, or the second lysine residue, or the third lysine residue, or the first and the second lysine residues, or the first and the third lysine residues, or the second and the third lysine residues, or the first, the second and the third lysine residues of the branched amino acid probe are each modified by attaching to the s-amino group(s) a molecule independently selected from the group consisting of Lys_{q}-Lys; (aa₃)ₚ-Lys_{q}; Lys_{q}-(aa₃)ₚ; [(aa₃)-Lys]ₚ and [Lys-(aa₃)]ₚ, wherein q is a number selected from 0, 1, 2 and 3; p is a number selected from 1, 2 and 3, and (aa₃) is an amino acid residue independently selected from Arg, His, Gly and Ala. In one embodiment the N-terminal Lys or (aa)₃ of the molecule is acetylated at the alpha amino group.

In a particular embodiment of the present disclosure (aa₃) is an amino acid residue independently selected from Gly and Ala. In a further embodiment, (aa₃) is Gly.

In one embodiment of the present disclosure the molecules to be covalently linked to the side chain amino group(s) of said first, second and/or third alkyl amine amino acid residue are acetylated at the alpha amino group of the N-terminal amino acid residue.

In one embodiment of the present disclosure the molecules are independently selected from the group consisting of Ac-AAA_{q}-AAA; Ac-(aa₃)ₚ-AAA_{q}; Ac-AAA_{q}-(aa₃)ₚ; Ac-[(aa₃)-AAA]ₚ and Ac-[AAA-(aa₃)]ₚ; and/or AAA_{q}-AAA; (aa₃)ₚ-AAA_{q}; AAA_{q}-(aa₃)ₚ; [(aa₃)-AAA]ₚ and [AAA-(aa₃)]ₚ.

In one embodiment of the present disclosure the molecules are independently selected from the group consisting of Ac-Orn_{q}-Orn; Ac-(aa₃)ₚ-Orn_{q}; Ac-Orn_{q}-(aa₃)ₚ; Ac-[(aa₃)-Orn]ₚ; Ac-[Orn-(aa₃)]ₚ; Ac-Ornₚ-Lysₚ; Ac-Lysₚ-Ornₚ; Ac-[Orn-Lys]ₚ and Ac-[Lys-Orn]ₚ; and/or Orn_{q}-Orn; (aa₃)ₚ-Orn_{q}; Orn_{q}-(aa₃)ₚ; [(aa₃)-Orn]ₚ and [Orn-(aa₃)]ₚ; Ornₚ-Lysₚ; Lysₚ-Ornₚ; [Orn-Lys], and [Lys-Orn]ₚ.

It follows that the molecules are in one embodiment of the present disclosure independently selected from the group consisting of Ac-Lys_{q}-Lys; Ac-(aa₃)ₚ-Lys_{q}; Ac-Lys_{q}-(aa₃)ₚ; Ac-[(aa₃)-Lys]ₚ and Ac-[Lys-(aa₃)]ₚ; and/or Lys_{q}-Lys; (aa₃)ₚ-Lys_{q}; LyS_{q}-(aa₃)ₚ; [(aa₃)-Lys]ₚ and [Lys-(aa₃)]ₚ.

In a particular embodiment of the present disclosure the molecule to be covalently linked to the side chain amino group(s) is Ac-AAA_{q}-AAA or AAA_{q}-AAA, wherein q is a number selected from 0, 1, 2 and 3.

It follows that in one embodiment of the present disclosure the branched amino acid probe consists of 2 to 9 amino alkyl amino acid residues. In one embodiment said 2 to 9 amino alkyl amino acid residues are individually selected from the group consisting of lysine, D-lysine, ornithine and D-ornithine.

In a particular embodiment of the present disclosure the molecule to be covalently linked to the side chain amino group(s) is Ac-Lys_{q}-Lys or Lys_{q}-Lys, wherein q is a number selected from 0, 1, 2 and 3.

It follows that in one embodiment of the present disclosure the branched amino acid probe consists of 2 to 9 lysine residues.

In one embodiment of the present disclosure the branched amino acid probe comprises a maximum of 1, 2, 3 or 4 amino acids selected from Arg, His, Gly and Ala (aa₃), wherein the remaining amino acids are amino alkyl amino acid residues. In another embodiment, the branched amino acid probe comprises a maximum of 1 Arg residue, and/or comprises a maximum of 1 His residue, and/or comprises a maximum of 1 Gly residue, and/or comprises a maximum of 1 Ala residue.

In one embodiment of the present disclosure the molecule to be covalently linked to the side chain amino group(s) of one or more of the first, second and/or third amino alkyl amino acid residues is selected from the group consisting of AAA, Ac-AAA, AAA-AAA, Ac-AAA-AAA, AAA-AAA-AAA, Ac-AAA-AAA-AAA, AAA-AAA-AAA-AAA, Ac-AAA-AAA-AAA-AAA, AAA-Gly-AAA, Ac-AAA-Gly-AAA, AAA-AAA-Gly, Ac-AAA-AAA-Gly, AAA-Gly, Ac-AAA-Gly, AAA-Ala-AAA, Ac-AAA-Ala-AAA, AAA-AAA-Ala, Ac-AAA-AAA-Ala, AAA-Ala, Ac-AAA-Ala, AAA-His-AAA, Ac-AAA-His-AAA, AAA-AAA-His, Ac-AAA-AAA-His, AAA-His, Ac-AAA-His, AAA-Arg-AAA, Ac-AAA-Arg-AAA, AAA-AAA-Arg, Ac-AAA-AAA-Arg, AAA-Arg and Ac-AAA-Arg; wherein AAA is an amino alkyl amino acid residue as specified herein. The above-mentioned AAA, Gly, Ala, His and Arg amino acid residues may each be in the L- or D-conformation.

In one embodiment of the present disclosure the molecule to be covalently linked to the side chain amino group(s) of one or more of the first, second and/or third amino alkyl amino acid residues is selected from the group consisting of Lys, Ac-Lys, Lys-Lys, Ac-Lys-Lys, Lys-Lys-Lys, Ac-Lys-Lys-Lys, Lys-Lys-Lys-Lys, Ac-Lys-Lys-Lys-Lys, Lys-Gly-Lys, Ac-Lys-Gly-Lys, Lys-Lys-Gly, Ac-Lys-Lys-Gly, Lys-Gly, Ac-Lys-Gly, Lys-Ala-Lys, Ac-Lys-Ala-Lys, Lys-Lys-Ala, Ac-Lys-Lys-Ala, Lys-Ala, Ac-Lys-Ala, Lys-His-Lys, Ac-Lys-His-Lys, Lys-Lys-His, Ac-Lys-Lys-His, Lys-His, Ac-Lys-His, Lys-Arg-Lys, Ac-Lys-Arg-Lys, Lys-Lys-Arg, Ac-Lys-Lys-Arg, Lys-Arg and Ac-Lys-Arg.

In a particular embodiment of the present disclosure the molecule to be covalently linked to the s-amino group(s) of one or more of the first, second and/or third lysine residues is selected from the group consisting of Lys, Ac-Lys, Lys-Lys, Ac-Lys-Lys, Lys-Lys-Lys, Ac-Lys-Lys-Lys, Lys-Lys-Lys-Lys, Ac-Lys-Lys-Lys-Lys, Lys-Gly-Lys, Ac-Lys-Gly-Lys, Lys-Lys-Gly, Ac-Lys-Lys-Gly, Lys-Gly, Ac-Lys-Gly, Lys-Ala-Lys, Ac-Lys-Ala-Lys, Lys-Lys-Ala, Ac-Lys-Lys-Ala, Lys-Ala, Ac-Lys-Ala, Lys-His-Lys, Ac-Lys-His-Lys, Lys-Lys-His, Ac-Lys-Lys-His, Lys-His, Ac-Lys-His, Lys-Arg-Lys, Ac-Lys-Arg-Lys, Lys-Lys-Arg, Ac-Lys-Lys-Arg, Lys-Arg and Ac-Lys-Arg.

In a particular embodiment of the present disclosure the branched amino acid probe comprise or consist of a first lysine residue selected from Lys and D-Lys, said first lysine residue being optionally N-terminally acetylated or C-terminally amidated, wherein said first lysine residue is modified by attaching to the s-amino group of said first lysine residue a molecule selected from the group consisting of Lys, Ac-Lys, Lys-Lys, Ac-Lys-Lys, Lys-Lys-Lys, Ac-Lys-Lys-Lys, Lys-Lys-Lys-Lys, Ac-Lys-Lys-Lys-Lys, Lys-Gly-Lys, Ac-Lys-Gly-Lys, Lys-Lys-Gly, Ac-Lys-Lys-Gly, Lys-Gly, Ac-Lys-Gly, Lys-Ala-Lys, Ac-Lys-Ala-Lys, Lys-Lys-Ala, Ac-Lys-Lys-Ala, Lys-Ala, Ac-Lys-Ala, Lys-His-Lys, Ac-Lys-His-Lys, Lys-Lys-His, Ac-Lys-Lys-His, Lys-His, Ac-Lys-His, Lys-Arg-Lys, Ac-Lys-Arg-Lys, Lys-Lys-Arg, Ac-Lys-Lys-Arg, Lys-Arg and Ac-Lys-Arg.

In a particular embodiment of the present disclosure the branched amino acid probe comprise or consist of a first and a second lysine residue each selected from Lys and D-Lys, said second lysine residue being optionally N-terminally acetylated or C-terminally amidated, wherein i) said first lysine residue, ii) said second lysine residue, or iii) said first and second residue are each modified by attaching to the s-amino group of said lysine residue a molecule selected from the group consisting of Lys, Ac-Lys, Lys-Lys, Ac-Lys-Lys, Lys-Lys-Lys, Ac-Lys-Lys-Lys, Lys-Lys-Lys-Lys, Ac-Lys-Lys-Lys-Lys, Lys-Gly-Lys, Ac-Lys-Gly-Lys, Lys-Lys-Gly, Ac-Lys-Lys-Gly, Lys-Gly, Ac-Lys-Gly, Lys-Ala-Lys, Ac-Lys-Ala-Lys, Lys-Lys-Ala, Ac-Lys-Lys-Ala, Lys-Ala, Ac-Lys-Ala, Lys-His-Lys, Ac-Lys-His-Lys, Lys-Lys-His, Ac-Lys-Lys-His, Lys-His, Ac-Lys-His, Lys-Arg-Lys, Ac-Lys-Arg-Lys, Lys-Lys-Arg, Ac-Lys-Lys-Arg, Lys-Arg and Ac-Lys-Arg.

In a particular embodiment of the present disclosure the branched amino acid probe comprise or consist of a first, a second and a third lysine residue each selected from Lys and D-Lys, said third lysine residue being optionally N-terminally acetylated or C-terminally amidated, wherein i) said first lysine residue, ii) said second lysine residue, iii) said third lysine residue, iv) said first and second lysine residue, v) said first and third lysine residue, vi) said second and third lysine residue, or vii) said first, second and third lysine residues are each modified by attaching to the s-amino group of said lysine residue a molecule selected from the group consisting of Lys, Ac-Lys, Lys-Lys, Ac-Lys-Lys, Lys-Lys-Lys, Ac-Lys-Lys-Lys, Lys-Lys-Lys-Lys, Ac-Lys-Lys-Lys-Lys, Lys-Gly-Lys, Ac-Lys-Gly-Lys, Lys-Lys-Gly, Ac-Lys-Lys-Gly, Lys-Gly, Ac-Lys-Gly, Lys-Ala-Lys, Ac-Lys-Ala-Lys, Lys-Lys-Ala, Ac-Lys-Lys-Ala, Lys-Ala, Ac-Lys-Ala, Lys-His-Lys, Ac-Lys-His-Lys, Lys-Lys-His, Ac-Lys-Lys-His, Lys-His, Ac-Lys-His, Lys-Arg-Lys, Ac-Lys-Arg-Lys, Lys-Lys-Arg, Ac-Lys-Lys-Arg, Lys-Arg and Ac-Lys-Arg.

In one embodiment of the present disclosure the branched amino acid probe comprises or consists of the formula: Ac—(Ac-Lys-Lys)Lys₁-(identical to Ac—(Ac-Lys-Lys)Lys-), wherein Lys₁ is the first lysine residue, which is acetylated, covalently linked to the N-terminus of a polypeptide as defined herein, and (Ac-Lys-Lys) is the molecule covalently linked to the s-amino group of said first lysine residue Lys₁.

In one embodiment of the present disclosure Ac—(Ac-Lys-Lys)Lys- is covalently linked to the N-terminal of the polypeptide as defined herein and/or to the side chain amino group of an amino alkyl amino acid residue within said polypeptide.

In one embodiment of the present disclosure the branched amino acid probe comprises or consists of the formula: Ac—(Ac-Lys)Lys₁-.

In one embodiment of the present disclosure the branched amino acid probe comprises or consists of the formula: (Ac-Lys-Lys)Lys₁-NH₂ (identical to (Ac-Lys-Lys)Lys-NH₂), wherein Lys₁ is the first lysine residue, which is amidated (—NH₂) at the C-terminal, and (Ac-Lys-Lys) is the molecule attached to the s-amino group of said first lysine residue Lys₁. In one embodiment of the present disclosure (Ac-Lys-Lys)Lys₁-NH₂ is attached to the C-terminal of the polypeptide as defined herein.

In one embodiment of the present disclosure the branched amino acid probe comprises or consists of a formula selected from the group consisting of (AAA) AAA₁-, (AAA-AAA) AAA₁-, (AAA-AAA-AAA) AAA₁-, (AAA-AAA-AAA-AAA) AAA₁-, (AAA-Gly-AAA) AAA₁-, (AAA-AAA-Gly) AAA₁-, (AAA-Gly) AAA₁-, (AAA-Ala-AAA) AAA₁-, (AAA-AAA-Ala) AAA₁-, (AAA-Ala) AAA₁-, (AAA-His-AAA) AAA₁-, (AAA-AAA-His) AAA₁-, (AAA-His) AAA₁-, (AAA-Arg-AAA) AAA₁-, (AAA-AAA-Arg) AAA₁-, and (AAA-Arg) AAA₁-. In one embodiment of the present disclosure said first amino alkyl amino acid reside (AAA₁-) is N-terminally acetylated or C-terminally amidated.

In one embodiment of the present disclosure the branched amino acid probe comprises or consists of a formula selected from the group consisting of (Lys)Lys₁-, (Lys-Lys)Lys₁-, (Lys-Lys-Lys)Lys₁-, (Lys-Lys-Lys-Lys)Lys₁-, (Lys-Gly-Lys)Lys₁-, (Lys-Lys-Gly) Lys₁-, (Lys-Gly) Lys₁-, (Lys-Ala-Lys)Lys₁-, (Lys-Lys-Ala) Lys₁-, (Lys-Ala) Lys₁-, (Lys-His-Lys)Lys₁-, (Lys-Lys-His)Lys₁-, (Lys-His)Lys₁-, (Lys-Arg-Lys)Lys₁-, (Lys-Lys-Arg) Lys₁-, and (Lys-Arg) Lys₁-. In one embodiment of the present disclosure said first lysine reside (Lys₁-) is N-terminally acetylated or C-terminally amidated.

In one embodiment of the present disclosure the branched amino acid probe comprises or consists of a formula selected from the group consisting of Ac—(Ac-Lys)Lys₁-, Ac—(Ac-Lys-Lys)Lys₁-, Ac—(Ac-Lys-Lys-Lys)Lys₁-, Ac—(Ac-Lys-Lys-Lys-Lys)Lys₁-, Ac—(Ac-Lys-Gly-Lys)Lys₁-, Ac—(Ac-Lys-Lys-Gly) Lys₁-, Ac—(Ac-Lys-Gly) Lys₁-, Ac—(Ac-Lys-Ala-Lys)Lys₁-, Ac—(Ac-Lys-Lys-Ala) Lys₁-, Ac—(Ac-Lys-Ala) Lys₁-, Ac—(Ac-Lys-His-Lys)Lys₁-, Ac—(Ac-Lys-Lys-His)Lys₁-, Ac—(Ac-Lys-His)Lys₁-, Ac—(Ac-Lys-Arg-Lys)Lys₁-, Ac—(Ac-Lys-Lys-Arg) Lys₁-, and Ac—(Ac-Lys-Arg) Lys₁-.

In one embodiment of the present disclosure the branched amino acid probe comprises or consists of a formula selected from the group consisting of (Ac-Lys)Lys₁-NH₂, (Ac-Lys-Lys)Lys₁-NH₂, (Ac-Lys-Lys-Lys)Lys₁-NH₂, (Ac-Lys-Lys-Lys-Lys)Lys₁-NH₂, (Ac-Lys-Gly-Lys)Lys₁-NH₂, (Ac-Lys-Lys-Gly) Lys₁-NH₂, (Ac-Lys-Gly) Lys₁-NH₂, (Ac-Lys-Ala-Lys)Lys₁-NH₂, (Ac-Lys-Lys-Ala) Lys₁-NH₂, (Ac-Lys-Ala) Lys₁-NH₂, (Ac-Lys-His-Lys)Lys₁-NH₂, (Ac-Lys-Lys-His)Lys₁-NH₂, (Ac-Lys-His)Lys₁-NH₂, (Ac-Lys-Arg-Lys)Lys₁-NH₂, (Ac-Lys-Lys-Arg) Lys₁-NH₂, and (Ac-Lys-Arg) Lys₁-NH₂.

More specifically, in one embodiment of the present disclosure the branched amino acid probe comprises or consists of a formula selected from the group consisting of Ac—(Ac-Lys)Lys₁-, Ac—(Ac-Lys-Lys)Lys₁-, Ac—(Ac-Lys-Lys-Lys)Lys₁-, Ac—(Ac-Lys-Lys-Lys-Lys)Lys₁-, Ac—(Ac-Lys-Gly-Lys)Lys₁-, Ac—(Ac-Lys-Lys-Gly) Lys₁- and Ac—(Ac-Lys-Gly) Lys₁-.

In one embodiment of the present disclosure the branched amino acid probe comprises or consists of the formula: Ac—(Ac-Lys)Lys₂-Lys₁-, wherein Lys₁ is the first lysine residue, Lys₂ is the second lysine residue which is acetylated and covalently linked to Lys₁ via a peptide bond, and (Ac-Lys) is the molecule covalently linked to the ε-amino group of said second lysine residue Lys₂.

In one embodiment of the present disclosure the branched amino acid probe comprises or consists of the formula: Ac-Lys₂-(Ac-Lys)Lys₁-, wherein the molecule (Ac-Lys) is covalently linked to the s-amino group of said first lysine residue Lys₁.

In one embodiment of the present disclosure the branched amino acid probe(s) is selected from the group consisting of

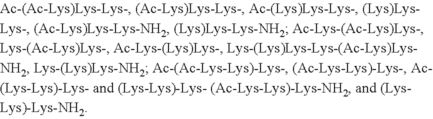

In one embodiment of the present disclosure the branched amino acid probe(s) is selected from the group consisting of Ac—(Ac-Lys)Lys-, Ac-(Lys)Lys-, (Ac-Lys)Lys-NH₂, (Lys)Lys-NH₂ and (Lys)Lys-.

In one embodiment of the present disclosure the branched amino acid probe is selected from the group consisting of Ac—(Ac-Lys)Lys₂-Lys₁-, Ac—(Ac-Lys-Lys)Lys₂-Lys₁-, Ac—(Ac-Lys-Gly) Lys₂-Lys₁-, Ac—(Ac-Lys-Lys-Lys)Lys₂-Lys₁-, Ac—(Ac-Lys-Lys-Lys-Lys)Lys₂-Lys₁-, Ac-Lys₂-(Ac-Lys)-Lys₁-, Ac-Lys₂-(Ac-Lys-Lys)-Lys₁-, Ac-Lys₂-(Ac-Lys-Gly)-Lys₁-, Ac-Lys₂-(Ac-Lys-Lys-Lys)-Lys₁-, Ac-Lys₂-(Ac-Lys-Lys-Lys-Lys)-Lys₁-, Ac—(Ac-Lys)Lys₂-(Ac-Lys-)-Lys₁-, Ac—(Ac-Lys)Lys₂-(Ac-Lys-Lys-)-Lys₁-, and Ac—(Ac-Lys-Lys)Lys₂-(Ac-Lys-Lys-)-Lys₁-.

More specifically, in one embodiment of the present disclosure the branched amino acid probe is selected from the group consisting of Ac—(Ac-Lys)Lys₂-Lys₁-, Ac—(Ac-Lys-Lys)Lys₂-Lys₁-, Ac—(Ac-Lys-Gly) Lys₂-Lys₁-, Ac-Lys₂-(Ac-Lys)-Lys₁-, Ac-Lys₂-(Ac-Lys-Lys)-Lys₁-, Ac-Lys₂-(Ac-Lys-Gly)-Lys₁-, Ac—(Ac-Lys)Lys₂-(Ac-Lys-)-Lys₁-, Ac—(Ac-Lys)Lys₂-(Ac-Lys-Lys-)-Lys₁-, and Ac—(Ac-Lys-Lys)Lys₂-(Ac-Lys-Lys-)-Lys₁-.

In one embodiment of the present disclosure the branched amino acid probe is selected from the group consisting of Ac-Lys₃-Lys₂-(Ac-Lys)Lys₁-, Ac-Lys₃-(Ac-Lys)Lys₂-Lys₁-, Ac—(Ac-Lys)Lys₃-Lys₂-Lys₁-, Ac-Lys₃-(Ac-Lys)Lys₂-(Ac-Lys)Lys₁-, Ac—(Ac-Lys)Lys₃-(Ac-Lys)Lys₂-Lys₁-, and Ac—(Ac-Lys)Lys₃-Lys₂-(Ac-Lys)Lys₁-.

In a particular embodiment of the present disclosure the branched amino acid probe is selected from the group consisting of Ac—(Ac-Lys)Lys₁-, Ac—(Ac-Lys-Lys)Lys₁-, Ac—(Ac-Lys-Lys-Lys)Lys₁-, Ac—(Ac-Lys-Lys-Lys-Lys)Lys₁-, Ac—(Ac-Lys-Gly-Lys)Lys₁-, Ac—(Ac-Lys-Lys-Gly) Lys₁-, Ac—(Ac-Lys-Gly) Lys₁-, Ac—(Ac-Lys)Lys₂-Lys₁-, Ac—(Ac-Lys-Lys)Lys₂-Lys₁-, Ac—(Ac-Lys-Gly) Lys₂-Lys₁-, Ac-Lys₂-(Ac-Lys)-Lys₁-, Ac-Lys₂-(Ac-Lys-Lys)-Lys₁-, Ac-Lys₂-(Ac-Lys-Gly)-Lys₁-, Ac—(Ac-Lys)Lys₂-(Ac-Lys-)-Lys₁-, Ac—(Ac-Lys)Lys₂-(Ac-Lys-Lys-)-Lys₁-, Ac—(Ac-Lys-Lys)Lys₂-(Ac-Lys-Lys-)-Lys₁-, Ac-Lys₃-Lys₂-(Ac-Lys)Lys₁-, Ac-Lys₃-(Ac-Lys)Lys₂-Lys₁-, Ac—(Ac-Lys)Lys₃-Lys₂-Lys₁-, Ac-Lys₃-(Ac-Lys)Lys₂-(Ac-Lys)Lys₁-, Ac—(Ac-Lys)Lys₃-(Ac-Lys)Lys₂-Lys₁-, and Ac—(Ac-Lys)Lys₃-Lys₂-(Ac-Lys)Lys₁. In one embodiment said branched amino acid probe is covalently linked to the N-terminal of the polypeptide as defined herein and/or to the side chain amino group of an amino alkyl amino acid residue within said polypeptide.

In a particular embodiment of the present disclosure the branched amino acid probe consists of 2 or 3 lysine residues (selected from Lys and D-Lys).

In a particular embodiment of the present disclosure the branched amino acid probe consists of 3 lysine residues. In another embodiment, the branched amino acid probe consists of 2 lysine residues.

In a particular embodiment of the present disclosure the branched amino acid probe consists of a first and a second lysine residue selected from Lys and D-Lys, wherein one or both of the first and second lysine residues are modified by attaching to the ε-amino group of said first and/or second lysine residue one lysine residue selected from Lys and D-Lys; wherein each of said lysine residues are optionally acetylated at the alpha amino group.

In a particular embodiment of the present disclosure the branched amino acid probe consists of a first lysine residue selected from Lys and D-Lys, wherein said first lysine residue is modified by attaching to the s-amino group of said first lysine residue two lysine residues selected from Lys and D-Lys; wherein each of said lysine residues are optionally acetylated at the alpha amino group.

### Linking the BAPs and the Polypeptides

According to the present disclosure, the first amino alkyl amino acid residue of each of the one or more branched amino acid probes is covalently linked to the N-terminus of a polypeptide or polypeptide analogue as defined herein, covalently linked to the C-terminus of said polypeptide or polypeptide analogue, and/or covalently linked to the side chain amino group of an amino alkyl amino acid residue within said polypeptide or polypeptide analogue.

Attaching one or more branched amino acid probes to a polypeptide or polypeptide analogue as defined herein yields a polypeptide/BAP-conjugate, such as polypeptide analogue/BAP-conjugate.

The term covalently linked to the N-terminus of said polypeptide or polypeptide analogue means that the first amino alkyl amino acid residue of the branched amino acid probe is covalently linked to the alpha amino group of the most N-terminal amino acid residue of the polypeptide or polypeptide analogue.

The term covalently linked to the C-terminus of said polypeptide or polypeptide analogue means that the alpha amino group of the first amino alkyl amino acid residue of the branched amino acid probe is covalently linked to the most C-terminal amino acid residue of the polypeptide or polypeptide analogue.

Furthermore, it is understood that a branched amino acid probe in one embodiment of the present disclosure is covalently linked to the side chain amino group of an amino alkyl amino acid residue within said polypeptide or polypeptide analogue.

In one particular embodiment of the present disclosure said amino alkyl amino acid residue within said polypeptide or polypeptide analogue sequence is selected from the group consisting of an ornithine residue and a lysine residue. In one particular embodiment of the present disclosure said amino alkyl amino acid residue within said peptide sequence is a lysine residue.

In one embodiment of the present disclosure the first amino alkyl amino acid residue of the branched amino acid probe is covalently linked to the δ-amino group of an ornithine residue within said polypeptide or polypeptide analogue or the s-amino group of a lysine residue within said polypeptide or polypeptide analogue.

In one embodiment of the present disclosure the first amino alkyl amino acid residue of the branched amino acid probe is covalently linked to the s-amino group of a lysine residue within said polypeptide or polypeptide analogue.

It is understood that an amino alkyl amino acid residue within said peptide sequence means that the amino alkyl amino acid residue does not form part of the branched amino acid probe per se, but is a residue occurring within the existing amino acid sequence of the polypeptide or polypeptide analogue. Said amino alkyl amino acid residue can be positioned at any position of the polypeptide or polypeptide analogue.

According to the present disclosure, a polypeptide analogue comprising one or more branched amino acid probes means that the polypeptide analogue in one embodiment comprises 1 branched amino acid probe, such as 2 branched amino acid probes, for example 3 branched amino acid probes, such as 4 branched amino acid probes, for example 5 branched amino acid probes, such as 6 branched amino acid probes.

The polypeptide analogue of the present disclosure can comprise any number of branched amino acid probes provided they can be covalently linked to the peptide (N-terminally, C-terminally and/or one or more amino alkyl amino acid residues within said polypeptide).

In one embodiment of the present disclosure the polypeptide analogue comprises 1 branched amino acid probe.

In one embodiment of the present disclosure the polypeptide analogue comprises 1 branched amino acid probe, which branched amino acid probe is covalently bound to the N-terminus of the polypeptide.

In one embodiment the of the present disclosure the polypeptide analogue comprises 1 branched amino acid probe, which branched amino acid probe is covalently bound to the C-terminus of the polypeptide.

In one embodiment of the present disclosure the polypeptide analogue comprises 1 branched amino acid probe, which branched amino acid probe is covalently linked to the side chain amino group of an amino alkyl amino acid residue within said polypeptide.

In one embodiment of the present disclosure the polypeptide analogue comprises more than one (two or more) branched amino acid probe(s). In the embodiments wherein the polypeptide analogue comprises more than one branched amino acid probe it is understood that the more than one branched amino acid probes may individually be the same (identical) or different (non-identical).

In one embodiment of the present disclosure the polypeptide analogue comprises 2 branched amino acid probes.

In one embodiment of the present disclosure the polypeptide analogue comprises 2 branched amino acid probes, wherein one branched amino acid probe is covalently bound to the N-terminus of the polypeptide and another branched amino acid probe is covalently bound to the C-terminus of the polypeptide.

In one embodiment of the present disclosure the polypeptide analogue comprises 2 branched amino acid probes, wherein one branched amino acid probe is covalently bound to the N-terminus of the polypeptide and another branched amino acid probe is covalently linked to the side chain amino group of an amino alkyl amino acid residue within said polypeptide.

In one embodiment of the present disclosure the polypeptide analogue comprises 2 branched amino acid probes, wherein one branched amino acid probe is covalently bound to the C-terminus of the polypeptide and another branched amino acid probe is covalently linked to the side chain amino group of an amino alkyl amino acid residue within said polypeptide.

In one embodiment of the present disclosure the polypeptide analogue comprises 2 branched amino acid probes, wherein each of the two branched amino acid probes are covalently linked to the side chain amino group of different (or separate) amino alkyl amino acid residues within said polypeptide.

In one embodiment of the present disclosure the polypeptide analogue comprises 3 branched amino acid probes.

In one embodiment of the present disclosure the polypeptide analogue comprises 3 branched amino acid probes, wherein the first branched amino acid probe is covalently bound to the N-terminus of the polypeptide, the second branched amino acid probe is covalently bound to the C-terminus of the polypeptide and the third branched amino acid probe is covalently linked to the side chain amino group of an amino alkyl amino acid residue within said polypeptide.

In one embodiment of the present disclosure the polypeptide analogue comprises 3 branched amino acid probes, wherein the first branched amino acid probe is covalently bound to the N-terminus of the polypeptide, and the second and third branched amino acid probes are each covalently linked to the side chain amino group of different amino alkyl amino acid residues within said polypeptide.

In one embodiment of the present disclosure the polypeptide analogue comprises 3 branched amino acid probes, wherein the first branched amino acid probe is covalently bound to the C-terminus of the polypeptide, and the second and third branched amino acid probes are each covalently linked to the side chain amino group of different amino alkyl amino acid residues within said polypeptide.

### Method of Treatment

The present disclosure in one embodiment relates to polypeptide analogues of Annexin A1, conjugated forms of said polypeptide analogues, as well as compositions comprising polypeptide analogues of Annexin A1 or their conjugated forms for use as medicaments and for the treatment of an ischemic condition and/or an inflammatory condition.

It is an aspect of the present disclosure to provide a polypeptide or polypeptide analogue selected from the group consisting of SEQ ID NO:2 to SEQ ID NO:24, SEQ ID NO: 25 to SEQ ID NO:47, and SEQ ID NO:48 to SEQ ID NO:70, or a functional variant thereof, for use as a medicament.

Furthermore, one aspect of the present disclosure relates to a polypeptide or polypeptide analogue selected from the group consisting of SEQ ID NO:2 to SEQ ID NO: 24, SEQ ID NO:25 to SEQ ID NO:47, and SEQ ID NO:48 to SEQ ID NO:70, or a functional variant thereof, for use in a method of treatment of an ischemic condition and/or an inflammatory condition.

In certain embodiments there is provided a polypeptide or polypeptide analogue selected from the group consisting of:

(SEQ ID NO: 50)
AMVSEFLKQAWFIENEEQEYVQTLKSSK,
(SEQ ID NO: 51)
AMVSEFLKQAWFIENEEQEYVQTLKSSKG,
(SEQ ID NO: 52)
AMVSEFLKQAWFIENEEQEYVQTLKSSKGG,
(SEQ ID NO: 53)
AMVSEFLKQAWFIENEEQEYVQTLKSSKGGP,
(SEQ ID NO: 54)
AMVSEFLKQAWFIENEEQEYVQTLKSSKGGPG,
(SEQ ID NO: 55)
AMVSEFLKQAWFIENEEQEYVQTLKSSKGGPGS,
(SEQ ID NO: 56)
AMVSEFLKQAWFIENEEQEYVQTLKSSKGGPGSA,
(SEQ ID NO: 57)
AMVSEFLKQAWFIENEEQEYVQTLKSSKGGPGSAV,
(SEQ ID NO: 58)
AMVSEFLKQAWFIENEEQEYVQTLKSSKGGPGSAVS,
(SEQ ID NO: 59)
AMVSEFLKQAWFIENEEQEYVQTLKSSKGGPGSAVSP,
and
(SEQ ID NO: 60)
AMVSEFLKQAWFIENEEQEYVQTLKSSKGGPGSAVSPY,

or a functional variant thereof,
for use in the treatment of an ischemic condition and/or an inflammatory condition.

In a preferred embodiment the polypeptide or polypeptide analogue is selected from the group of:

(SEQ ID NO: 50)
AMVSEFLKQAWFIENEEQEYVQTLKSSK,
(SEQ ID NO: 55)
AMVSEFLKQAWFIENEEQEYVQTLKSSKGGPGS,
and
(SEQ ID NO: 60)
AMVSEFLKQAWFIENEEQEYVQTLKSSKGGPGSAVSPY,

or a functional variant thereof,
for use in the treatment of an ischemic condition and/or an inflammatory condition.

Functional variants are disclosed herein elsewhere.

Furthermore, the present disclosure also relates to a polypeptide or polypeptide analogue selected from the group consisting of SEQ ID NO:2 to SEQ ID NO:24, SEQ ID NO: 25 to SEQ ID NO:47, and SEQ ID NO:48 to SEQ ID NO:70, or a functional variant thereof, for use in the manufacture of a medicament for the treatment of an ischemic condition and/or an inflammatory condition.

In another aspect, the present disclosure provides methods for treatment, prevention or alleviation of an ischemic condition and/or an inflammatory condition. Such methods according to the present disclosure in one embodiment comprise one or more steps of administration or release of an effective amount of a polypeptide or polypeptide analogue selected from the group consisting of SEQ ID NO:2 to SEQ ID NO:24, SEQ ID NO: 25 to SEQ ID NO:47, and SEQ ID NO:48 to SEQ ID NO:70, or a functional variant thereof, as disclosed herein, or a pharmaceutical composition comprising one or more such polypeptide or polypeptide analogue, to an individual in need thereof.

An individual in need as referred to herein, is in one embodiment an individual that benefits from the administration of a polypeptide or polypeptide analogue as disclosed herein or a pharmaceutical composition comprising said polypeptide or polypeptide analogue according to the present disclosure. Such an individual in one embodiment suffers from a disease or condition or is at risk of suffering therefrom. The individual is in one embodiment any human being, male or female, infant, middle-aged or old. The disorder to be treated or prevented in the individual in one embodiment relates to the age of the individual, the general health of the individual, the medications used for treating the individual and whether or not the individual has a prior history of suffering from diseases or disorders that may have or have induced the condition in the individual.

The terms “treatment” and “treating” as used herein refer to the management and care of a patient for the purpose of combating a condition, disease or disorder. The term is intended to include the full spectrum of treatments for a given condition from which the patient is suffering, such as administration of the polypeptide analogue of the present disclosure for the purpose of alleviating or relieving symptoms or complications, delaying the progression of the condition, partially arresting the clinical manifestations, disease or disorder; curing or eliminating the condition, disease or disorder; and/or preventing or reducing the risk of acquiring the condition, disease or disorder, wherein “preventing” or “prevention” is to be understood to refer to the management and care of a patient for the purpose of hindering the development of the condition, disease or disorder, and includes the administration of the active compounds to prevent or reduce the risk of the onset of symptoms or complications. The patient to be treated is preferably a mammal, in particular a human being. Treatment of animals, such as mice, rats, dogs, cats, cows, horses, sheep and pigs, is, however, also within the scope of the present disclosure. The patients to be treated according to the present disclosure can be of various ages, for example, adults, children, children under 16, children age 6-16, children age 2-16, children age 2 months to 6 years or children age 2 months to 5 years.

As used herein, reference to “inflammation” or “inflammatory response or disease or condition” refers to any inflammatory response or disease, including but not limited to inflammation of the eye, gout, gouty arthritis, rheumatoid arthritis, asthma, reperfusion injury or damage, stroke, myocardial infarction, septic shock, or an inflammatory skin disorder, such as psoriasis or eczema.

### Medical Indications

In one aspect the present disclosure provides a polypeptide or polypeptide analogue as disclosed herein for use in the treatment of an ischemic condition and/or an inflammatory condition.

In some embodiments of the present disclosure, said treatment is prophylactic, ameliorative or curative.

In some embodiments of the present disclosure, said ischemic condition and/or inflammatory condition is acute, subacute or chronic.

In one embodiment of the present disclosure there is provided a polypeptide or polypeptide analogue as disclosed herein for use in the treatment of an ischemic condition and/or an inflammatory condition in the tissue of one or more organs of a mammal.

In some embodiments of the present disclosure, said mammal is a human (Homo sapiens).

In some embodiments of the present disclosure, said organ is selected from the group consisting of kidney, liver, brain, heart, muscles, bone marrow, skin, skeleton, lungs, the respiratory tract, spleen, exocrine glands, bladder, endocrine glands, reproduction organs including the phallopian tubes, eye, ear, vascular system, the gastrointestinal tract including small intestines, colon, rectum, canalis analis and prostate gland.

In some embodiments of the present disclosure, said ischemic condition is secondary ischemia.

In some embodiments of the present disclosure, said ischemia is secondary ischemia and it is due to stroke, injury, septic shock, systemic hypotension, cardiac arrest due to heart attack, cardiac arrhythmia, atheromatous disease with thrombosis, embolism from the heart or from blood vessel from any organ, vasospasm, aortic aneurysm or aneurisms in other organs, coronary stenosis, myocardial infarction, angina pectoris, pericarditis, myocarditis, myxodemia, or endocarditis.

An aortic aneurysm is in one embodiment thoracic or abdominal or dissecting aortic aneurysm. Systemic hypotension is in one embodiment hypotension due to heart disease, hypotension due to systemic disease including infection or allergic reactions, or hypotension due to one or more toxic compound or poison(s) or drug(s).

In some embodiments of the present disclosure, said ischemic condition is myocardial ischemia.

In some embodiments of the present disclosure, said ischemic and/or inflammatory condition is associated with surgery, such as major surgery; or is associated with organ transplantation, such as solid organ transplantation.

In some embodiments of the present disclosure, said ischemic and/or inflammatory condition is selected from the group consisting of post-surgical systemic inflammatory response syndrome (SIRS) and post-surgical organ dysfunction; such as post-surgical renal failure including acute kidney injury (AKI), nephrotoxicity and/or chronic renal failure (CRF).

In some embodiments of the present disclosure, said ischemic and/or inflammatory condition is reperfusion injury.

In some embodiments of the present disclosure, said inflammatory disease is selected from the group consisting of arthropathy (joint disease), rheumatoid arthritis (RA), gout, inflammatory diseases of the gastrointestinal system, and multiple sclerosis.

In some embodiments of the present disclosure, said ischemic and/or inflammatory condition is inflammation of the liver. In one embodiment of the present disclosure, said inflammatory disease is Nonalcoholic Steatohepatitis (NASH).

In some embodiments of the present disclosure, said ischemic and/or inflammatory condition in the tissue of one or more organs is due to cardiac arrhythmia. In one embodiment of the present disclosure, said cardiac arrhythmia is the primary disease or secondary to another condition of the individual, including acute infections particularly those affecting the lungs, pulmonary embolism, hypotension, shock, anoxaemia and anaemia.

Cardiac arrhythmias include ventricular or supra ventricular tachyarrhythmias, atrioventricular block, sinus node disease, Wolff-Parkinson-White syndrome, Lenegres disease, Lev's disease any syndrome involving an abnormal myocardial connection between atrium and ventricle.

In some embodiments of the present disclosure, secondary ischemia can also be observed in connection with a range of other diseases and conditions, including but not limited to diabetes mellitus, hyperlipidaemia, thromboangiitis obliterans, Takayasu's syndrome, arteritis temporalis, mucocutaneous lymph node syndrome (Kawasaki disease), cardiovascular syphilis, connective tissue disorders such as Raynaud's disease, phlegmasia coerulae dolens, blood vessel trauma including iatrogene trauma such as cannulation, conditions with increased fasting levels of LDL-Cholesterol, triglycerid, and/or HDL-Cholesterol, retroperitoneal fibrosis, rheumatic diseases, systemic lupus erythematosus, polyarteritis nodosa, scleroderma, polymyositis, dermatomyositis, rheumatoid arthritis, neuromyopathic disorders such as progressive muscular dystrophy of Duchenne, Friedreich's ataxia, and myotonic dystrophy, anaphylaxis, serum sickness, hemolytic anaemia, allergy, and allergic agranulocytosis. In one embodiment the polypeptides and polypeptide analogues of the present disclosure are also useful in the treatment or prevention of said conditions.

In some embodiments of the present disclosure the condition to be treated is caused by a cancer or a by premalignant disorder having an impact on the organ, e.g. on the respiratory system including lung, bronchiole, upper airways, and/or on the heart and/or on the kidney and/or on the gastrointestinal system, including acute leukemia, chronic myelocytic leukemia, chronic lymphocytic leukemia, Hodgkin's disease, lymphosarcoma, myeloma, metastasizing carcinoma of any origin. In some embodiments of the present disclosure the polypeptides or polypeptide analogues disclosed herein are used in the treatment or prevention of said conditions.

In some embodiments of the present disclosure, the ischemic and/or inflammatory condition in the tissue of one or more organs is caused by a physical trauma including electromagnetic radiation.

One aspect of the present disclosure provides a method for treatment of an ischemic condition and/or an inflammatory condition comprising administering an effective amount of a polypeptide or polypeptide analogue as disclosed herein to an individual in need thereof.

### Surgery and Transplantation

Major surgical interventions including cardiothoracic surgery, abdominal surgery, surgery on the aorta and other major blood vessels, as well as organ transplantation such as lung or heart or combined lung and heart transplantation, liver transplantation or renal transplantation induce a systemic inflammatory response (SIR; or systemic inflammatory response syndrome SIRS) and is associated with post-surgical organ dysfunction including development of renal failure.

Renal failure is a consequence of the SIR and the reduced blood flow generated during the surgical intervention. The result is post-surgical acute kidney injury (AKI) which for a large fraction deteriorates into chronic renal failure. Currently no efficient treatment modality exists to prevent the development of renal failure. Post-surgical renal failure may be defined as a more than 25% reduction in Glomerular filtration rate (GFR) present 3 month after the surgical intervention.

Major cardiac surgery such as repair of one or more cardiac valves, cardiac artery bypass grafting (CABG), surgery on the aortic root, or aortic branch including the common carotic arteries, or combined cardiac surgery such as valve(s) replacement and CABG and/or aortic root surgery is associated with development of renal impairment that, when present, is associated with increased morbidity and mortality.

In some embodiments of the present disclosure, treatment with a polypeptide or polypeptide analogue disclosed herein reduces the degree of renal impairment. For example this is achieved by reducing the fall in GFR post-surgery; by reducing the degree of post-surgical increases in serum creatinine or cystatin C or the more immediate increases in urinary excretion of AKI markers NGAL, IL18 or KIM-1; and/or or by reducing the degree of post-surgical SIR (for example by reduced circulating levels of IL-6 and other pro-inflammatory markers).

Lung transplantation (LTX) is the ultimate treatment modality for end-stage lung disease. The major challenges associated with LTX are scarcity of donors, acute and chronic rejection of the transplanted lungs and side-effects of immune suppressive treatment including development of chronic renal failure (CRF).

While there has been a good development in the treatment of acute rejection by newer immunosuppressive drugs leading to fewer episodes of acute rejection within the first year, fewer organ losses, fewer side effects, fewer infections, and less invasive monitoring methods, the control of chronic organ rejection has not greatly improved and the half-life time in terms of how many years 50% of the patients survive has only marginally improved during the last 2 decades to around 7 years.

Side effects of the immunosuppressive treatment are dominated by 2 major challenges: Nephrotoxicity and post-transplant lymphoproliferative diseases (PTLD), where the latter can be considered as a consequence of the degree of immune-suppression needed to avoid chronic organ rejection-“too much” keeps the rejection on distance, but gives infections and PTLD, while giving “too little” puts the patients at an increased risk of rejecting the graft. Neprotoxicity and development of CRF is despite of extensive research during the last 30 years, still a significant problem. Five years after LTX none of the patients retain normal kidney function and 20% of the long term survivors will end with a kidney transplant as well.

Calcineurin inhibitor treatment (Tacrolimus, Cyclosporin A) is the corner-stone in the immune-suppressive treatment strategy for successful solid organ transplantation. The limiting factor in using calcineurin inhibitors is the acute and chronic irreversible nephrotoxicity. Recent data indicate that kidney function (measured as reduction in GFR) is reduced with 40% within the first 14 days after LTX and that this reduction is irreversible.

Heart transplantation (HTX) is the ultimate treatment modality for end-stage heart failure. As for LTX the major challenges associated with HTX are scarcity of donors, acute and chronic rejection of the transplanted hearts and side-effects of immune suppressive treatment including development of CRF. Like for LTX the number of patients with retained kidney function over time is limited or absent and like LTX a major reduction in kidney function is present already two to four weeks post transplantation.

This dramatic effect on kidney function seen after LTX and HTX is probably not caused by calcineurin inhibitor treatment alone, but is the final result of the surgical and anesthesiological trauma in combination with the organ ischemia and side effects of antibiotic, antiviral, antifungal and immunsuppressive drugs. Consequently, in one embodiment pharmacological intervention by employment of the polypeptides or polypeptide analogues of the present disclosure will reduce the degree of renal impairment associated with organ transplantation, such as LTX and HTX.

Surgery, as is outlined herein above in detail, including organ transplantation, may thus be the cause of secondary ischemia.

The present disclosure is thus in one embodiment directed to a polypeptide or polypeptide analogue as disclosed herein for use in the treatment of an ischemic and/or inflammatory condition in the tissue of one or more organs of a mammal, wherein said ischemic and/or inflammatory condition is associated with surgery. In one embodiment of the present disclosure said surgery is major surgery or major surgical intervention.

In one embodiment of the present disclosure, said surgery is selected from the group consisting of cardiothoracic surgery, abdominal surgery, surgery on the aorta and/or other major blood vessels, repair of one or more cardiac valves, cardiac artery bypass grafting (CABG), surgery on the aortic root or the aortic branch including the common carotic arteries, and combined cardiac surgery such as valve(s) replacement and CABG and/or aortic root surgery.

In one embodiment of the present disclosure, said surgery encompasses surgical insertion transplants, devices, grafts, prostheses or other biomedical compounds or devices inserted by surgical operations.

In one embodiment of the present disclosure, said major surgery comprises organ transplantation. It follows that the present disclosure in one embodiment is directed to a polypeptide or polypeptide analogue as disclosed herein for use in the treatment of an ischemic and/or inflammatory condition in the tissue of one or more organs of a mammal, wherein said ischemic and/or inflammatory condition is associated with organ transplantation. In one embodiment of the present disclosure, said organ transplantation is solid organ transplantation.

In one embodiment of the present disclosure said solid organ transplantation is heart transplantation, lung transplantation, combined heart and lung transplantation, liver transplantation or kidney (renal) transplantation.

The present disclosure in another embodiment is directed to a polypeptide or polypeptide analogue as disclosed herein for use in the treatment of post-surgical systemic inflammatory response syndrome (SIRS), post-surgical organ dysfunction and/or post-surgical renal failure such as acute kidney injury (AKI), neprotoxicity and/or chronic renal failure (CRF).

The present disclosure is in one embodiment directed to a polypeptide or polypeptide analogue as disclosed herein for reducing the degree of renal impairment associated with major surgery, in one embodiment organ transplantation.

Reperfusion injury is tissue damage caused when blood supply returns to the tissue after a period of ischemia or lack of oxygen. The absence of oxygen and nutrients from blood during the ischemic period creates a condition in which the restoration of circulation results in inflammation and oxidative damage through the induction of oxidative stress rather than restoration of normal function.

Reperfusion injuries may occur in connection with surgery, such as major surgical interventions including organ transplantations. It is a primary concern when performing liver transplantations, and also during cardiac surgery.

In a particular embodiment of the present disclosure, said ischemic and/or inflammatory condition in the tissue of one or more organs is associated with reperfusion injury. Thus, in one embodiment the present disclosure is directed to a polypeptide or polypeptide analogue as disclosed herein for use in the treatment of an ischemic and/or inflammatory condition in the tissue of one or more organs of a mammal, wherein said ischemic and/or inflammatory condition is associated with reperfusion injury.

In some embodiments of the present disclosure, the polypeptides, polypeptide analogues or compositions of the present disclosure are to be administered before and/or during surgery and/or organ transplantation.

In some embodiments of the present disclosure the ischemic and/or inflammatory condition in the tissue of one or more organs as described herein is caused by toxin- or drug-induced cell, tissue or organ failure.

### Toxins and Drugs

The present disclosure is in one embodiment directed to a polypeptide or polypeptide analogue as disclosed herein for use in the treatment of an ischemic and/or inflammatory condition in the tissue of one or more organs of a mammal, wherein said ischemic and/or inflammatory condition is caused (or induced) by toxin- or drug-induced cell, tissue or organ failure.

Said drug includes but are not restricted to cancer chemotherapeutics including cisplatin, carboplatin, dacarbezine, procarbazine, altretamine, semustine, lomustine, carmustine, busulfan, thiotepa, melphalan, cyclophosphamide, chlorambucil, mechlorethamine, azadtidine, cladrrbine, cytorabine, fludarabine, fluorouracil, mercaptopurine, metrotrexate, thioguanine, allopurinol, bleomycin, dactinomycin, daunorubicin, docetaxel, doxorubicin (adriamycin), etoposide, idarubicin, irinotecan, mitomycin, paclitaxel, plicamycin, topotecan, vinblastine, vincristine, vinorelbine, amasacrine, asparaginase, hydroxyurea, mititane, mitoxantrone; Antibiotics as aminoglycosides including streptomycin, neomycin, kanamycin, amikacin, gentamicin, tobramycin, sisomicin and nitilmicin; immunodepressive compounds as cyclosporine; tricyclic antidepressants, lithium salts, prenylamine and phenothizine derivatives.

### Inflammatory Conditions

Inflammation is a localized defensive response of the body against pathogens and injury. Immune cells and soluble factors take part in this process to neutralize the injurious agent and initiate tissue repair to restore homeostasis. Loss of regulation of these mechanisms can prevent the final resolution of the inflammatory process, leading to chronic inflammation. Chronic inflammation is extremely relevant in today's modern medicine, as it contributes to the pathogenesis of the most important diseases of the industrialized societies including atherosclerosis, acute and chronic heart failure, cancer, diabetes, and obesity-associated diseases. Recent insight into endogenous anti-inflammatory pathways have identified a number of natural anti-inflammatory and pro-resolving molecules and pathways suitable for pharmacological intervention that would make it possible to develop drugs that mimic the natural course of resolving inflammation. Among these natural anti-inflammatory and pro-resolving pathways is Annexin A1 acting through FPR2 stimulation.

The immune modulating effects of FPR-2 agonists are exerted through inhibition of inflammatory mediators and by inhibition of inflammatory cell migration. FPR-2 agonists exert these effects in a variety of cells including monocytes, macrophages, subtypes of T-cells, endothelial cells and epithelial cells.

Joint diseases such as rheumatoid arthritis (RA) and gout are characterized by episodes with acute exacerbations, in RA the exacerbations (often described as flairs) typically develop on top of chronic symptoms and develop despite intense pharmacological treatment. A similar pattern can be seen in gout, with the major difference that most gout patients are without symptom between the exacerbations. In both conditions significant neutrophil infiltration into the synovial membrane and joint fluid are the primary pathological hallmark of the exacerbations. The most important pro-inflammatory effectors involved include IL-1B, TNF-α, IL-6, IL-8, and COX-2. Resolution of the acute exacerbations to avoid development or deterioration of chronic inflammation involves activation of macrophages to phagocyte the apoptotic neutrophils.

Consequently in one embodiment of the present disclosure it would be attractive to apply treatment with a polypeptide or polypeptide analogue as disclosed herein to joint diseases, not at least in order to reduce the severity of exacerbations in existing disease as flairs in rheumatoid arthritis would have major clinical impact. However, not only joint diseases are associated with exacerbations of symptoms. Neurodegenerative diseases such as multiple sclerosis have flair-like exacerbations where treatment with a polypeptide or polypeptide analogue as disclosed herein in one embodiment of the present disclosure could reduce the symptoms and eventually as for joint diseases reduce the overall deterioration of the patients' functional level.

The present disclosure is in one embodiment directed to a polypeptide or polypeptide analogue as disclosed herein for use in the treatment of an inflammatory condition in the tissue of one or more organs of a mammal, wherein said ischemic and/or inflammatory condition is an inflammatory disease.

In one embodiment of the present disclosure, said inflammatory disease is Arthritis. In one embodiment of the present disclosure, said inflammatory disease is selected from the group consisting of an arthropathy (a disease of a joint, Arthritis (including diseases associated with arthritis), osteoartritis, rheumatoid arthritis; spondylarthropathies (e.g. ankylosing spondilitis), reactive arthritis (including arthritis following rheumatic fever), Henoch-Schonlein purpura, Reiter's disease, Juvenile Chronic arthritis including Still's disease, juvenile rheumatoid arthritis, juvenile ankylosing spondylitis, psoriasis, osteoarthritis, osteoarthritis secondary to hypermobilty, congenital dysplasias, slipped femoral epiphysis, Perthes' disease, intra-articular fractures, meniscectomy, obesity, recurrent dislocation, repetitive actions, crystal depositions and diseases and metabolic abnormalities of cartilage including pyrophosphate arthropathy, ochronosis, haemochromatosis, avascular necrosis including Sickle Cell disease, therapy with corticoids or other drugs, Caisson disease, septic or infectious arthitis (including tuberculous arthritis, meningococcal arthritis, gonococcal arthritis, salmonella arthritis), infective endocarditis, viral arthritis, recurrent haemarthrosis, and all kinds of deposition diseases such as Gout, pyrophosphate arthopathy and acute calcific periarthritis.

In one embodiment of the present disclosure, said inflammatory disease is a connective tissue disorder; in one embodiment selected from the group consisting of systemic lupus erythematosus, polymyositis/dermatomyositis, systemic sclerosis, mixed connective tissue disease, sarcoidosis and primary Sjogrens syndrome including keratoconjunctivitis sicca, polymyalgia rheumatica, and other types of vasculitis, crystal deposition diseases (including gout), pyrophosphate arthropathy, and acute calcific periarthritis.

In one embodiment of the present disclosure, said inflammatory disease is a soft-tissue rheumatism including bursitis, tenosynovitis or peritendonitis, enthesitis, nerve compression, periarthritis or capsulitis, muscle tension and muscle dysfunction.

In one embodiment of the present disclosure, said inflammatory disease is selected from the group consisting of vasculitis including vasculitis secondary to rheumatoid arthritis, infective vasculitis due to infections with bacterial species including spirochaetal diseses as Lyme disease, syphilis, rickettsial and mycobacterial infections, fungal, viral or protozoal infections, non-infective vasculitis secondary to hypersensibility and leucocytoplastic vasculitis including Serum Sickness and Henoch-Schonlein purpura, Drug induced vasculitis, essential mixed cryoglobulinaemia, hypocomplentaemia, Vasculitis associated with other kinds of malignancy, non-infective vascultitis including Takayasu's arteritis/disease, Giant Cell Arteritis (Temporal arteritis and polymyalgia rheumatica), Buerger's disease, polyarteritis nodosa, microscopic polyarteritis, Wegener's granulomatose, Churg-Strauss syndrome, and vasculitis secondary to connective tissue diseases including Systemic Lupus Erythematosus, Polymyositis/Dermatomyositis, Systemic Sclerosis, Mixed Connetive Tissue Disease, sarcoidosis and Primary Sjogrens syndrome.

In one embodiment of the present disclosure, said inflammatory disease is inflammatory diseases of the gastrointestinal system. Said inflammatory diseases of the gastrointestinal system may be selected from the group consisting of inflammatory bowel disease, coeliac disease, gluten sensitive enteropathy, eosinophilic gastroenteritis, intestinal lympangiectasia, inflammatory bowel disease (including Chrohn's disease and ulcerative colitis), diverticular disease of the colon, radiation enteritis, irritable bowel syndrome, Whipple's diease, stomatitis of all kinds, salivary gland diseases (such as sarcoidosis, salivary duct obstruction and Sjogrens syndrome), inflammaton of the oesophagus (e.g. due to gastro-oesophagel reflux or infections with Candida species, herpes simplex and cytomegalus virus), inflammatory diseases of the stomach (including acute and chronic gastritis, Helicobacter pylori infection and Mentriers disease), and inflammation of the small intestine.

In one embodiment of the present disclosure, said inflammatory disease is a neurodegenerative disease, such as a neurodegenerative disease having an inflammatory component, such as multiple sclerosis (MS).

In one embodiment of the present disclosure, said inflammatory disease is selected from the group consisting of dermatitis, pemfigus, bulloid pemphigoid, benign mucous membrane pemphigoid, dermatitis herpitiformis, tropical sprue, systemic amyloidosis, primary biliary cirrhosis, Goodpasture syndrome, all kinds of deposition diseases as Gout, pyrophosphate arthopathy and acute calcific periarthritis, pancreatitis, septic discitis, tuberculosis, malignancies (such as matastases, myeloma and others), spinal tumours, ancylosing spondylitis, acute disc prolapse, chronic disc disease/osteoarthritis, osteoporosis, and osteomalacia, Pagets disease, hyperparathyroidism, renal osteodystrophy, spondylolisthesis, spinal senosis congenital abnormalities and fibromyalgia.

In one embodiment of the present disclosure, said inflammatory disease is selected from the group consisting of upper and lower airway diseases such as chronic obstructive pulmonary diseases (COPD), allergic and non-allergic asthma, allergic rhinitis, allergic and non-allergic conjunctivitis, allergic and non-allergic dermatitis and lung inflammation.

### Further Active Ingredients

In some embodiments of the present disclosure, the polypeptides or polypeptide analogues disclosed herein are combined with or comprise one or more further active ingredients which are understood as other therapeutic compounds or pharmaceutically acceptable derivatives thereof.

Methods for treatment according to the present disclosure in one embodiment thus further comprise one or more steps of administration of one or more further active ingredients, either concomitantly or sequentially, and in any suitable ratios.

Methods of treatment according to the present disclosure in one embodiment include a step wherein the pharmaceutical composition or the polypeptide or polypeptide analogue as defined herein is administered simultaneously, sequentially or separately in combination with one or more further active ingredients.

### Administration and Dosage

According to the present disclosure, a composition comprising a polypeptide or polypeptide analogue selected from the group consisting of SEQ ID NO:2 to SEQ ID NO: 24, SEQ ID NO:25 to SEQ ID NO:47, and SEQ ID NO:48 to SEQ ID NO:70, or a functional variant thereof, is in one embodiment administered to individuals in need thereof in pharmaceutically effective doses or a therapeutically effective amount.

A therapeutically effective amount of a polypeptide or polypeptide analogue according to the present disclosure is in one embodiment an amount sufficient to cure, prevent, reduce the risk of, alleviate or partially arrest the clinical manifestations of a given disease or disorder and its complications. The amount that is effective for a particular therapeutic purpose will depend on the severity and the sort of the disorder as well as on the weight and general state of the subject. An amount adequate to accomplish this is defined as a “therapeutically effective amount”.

In one embodiment the composition is administered in doses of from 1 μg/day to 100 mg/day; such as from 1 μg/day to 10 μg/day, such as 10 μg/day to 100 μg/day, such as 100 μg/day to 250 μg/day, such as 250 μg/day to 500 μg/day, such as 500 μg/day to 750 μg/day, such as 750 μg/day to 1 mg/day, such as 1 mg/day to 2 mg/day, such as 2 mg/day to 5 mg/day, or such as 5 mg/day to 10 mg/day, such as 10 mg/day to 20 mg/day, such as 20 mg/day to 30 mg/day, such as 30 mg/day to 40 mg/day, such as 40 mg/day to 50 mg/day, such as 50 mg/day to 75 mg/day, or such as 75 mg/day to 100 mg/day.

In one embodiment of the present disclosure, one single dose of the composition is administered and may comprise of from 1 μg/kg body weight to 100 mg/kg body weight; such as from 1 to 10 μg/kg body weight, such as 10 to 100 μg/day, such as 100 to 250 μg/kg body weight, such as 250 to 500 μg/kg body weight, such as 500 to 750 μg/kg body weight, such as 750 μg/kg body weight to 1 mg/kg body weight, such as 1 mg/kg body weight to 2 mg/kg body weight, such as 2 to 5 mg/kg body weight, such as 5 to 10 mg/kg body weight, such as 10 to 20 mg/kg body weight, such as 20 to 30 mg/kg body weight, such as 30 to 40 mg/kg body weight, such as 40 to 50 mg/kg body weight, such as 50 to 75 mg/kg body weight, or such as 75 to 100 mg/kg body weight.

In one embodiment, a dose according to the present disclosure is administered one or several times per day, such as from 1 to 6 times per day, such as from 1 to 5 times per day, such as from 1 to 4 times per day, such as from 1 to 3 times per day, such as from 1 to 2 times per day, such as from 2 to 4 times per day, such as from 2 to 3 times per day.

### Routes of Administration

It will be appreciated that the preferred route of administration will depend on the general condition and age of the subject to be treated, the nature of the condition to be treated, the location of the tissue to be treated in the body and the active ingredient chosen.

In one embodiment of the present disclosure, the route of administration allows for introducing the polypeptide or polypeptide analogue into the blood stream to ultimately target the sites of desired action.

In one embodiment of the present disclosure the routes of administration is any suitable routes, such as an enteral route (including the oral, rectal, nasal, pulmonary, buccal, sublingual, transdermal, intracisternal and intraperitoneal administration), and/or a parenteral route (including subcutaneous, intramuscular, intrathecal, intravenous and intradermal administration). Appropriate dosage forms may be prepared by conventional techniques.

Parenteral administration is any administration route not being the oral/enteral route whereby the medicament avoids first-pass degradation in the liver. Accordingly, parenteral administration includes any injections and infusions, for example bolus injection or continuous infusion, such as intravenous administration, intramuscular administration or subcutaneous administration. Furthermore, parenteral administration includes inhalations and topical administration.

Accordingly, the polypeptide or polypeptide analogue or composition as disclosed herein is in one embodiment of the present disclosure administered topically to cross any mucosal membrane of an animal to which the substance or peptide is to be given, e.g. in the nose, vagina, eye, mouth, genital tract, lungs, gastrointestinal tract, or rectum, for example the mucosa of the nose, or mouth, and accordingly, parenteral administration may also include buccal, sublingual, nasal, rectal, vaginal and intraperitoneal administration as well as pulmonary and bronchial administration by inhalation or installation. In some embodiments, the polypeptide or polypeptide analogue or composition as disclosed herein is administered topically to cross the skin.

In one embodiment of the present disclosure, the intravenous, subcutaneous and intramuscular forms of parenteral administration are employed.

In one embodiment of the present disclosure, the polypeptide or polypeptide analogue or composition as disclosed herein is used as a local treatment, i.e. is introduced directly to the site(s) of action. Accordingly, the polypeptide or polypeptide analogue or composition may be applied to the skin or mucosa directly, or the polypeptide or polypeptide analogue or composition may be injected into the site of action, for example into the diseased tissue or to an end artery leading directly to the diseased tissue.

### Pharmaceutical Formulations

In one embodiment of the present disclosure the polypeptides or polypeptide analogues or pharmaceutically acceptable derivatives thereof are administered alone or in combination with pharmaceutically acceptable carriers or excipients, in either single or multiple doses. The pharmaceutical compositions or polypeptides according to the disclosure may be formulated with pharmaceutically acceptable carriers or diluents as well as any other known adjuvants and excipients in accordance with conventional techniques, such as those disclosed in Remington: The Science and Practice of Pharmacy, 20th Edition, Gennaro, Ed., Mack Publishing Co., Easton, PA, 2000.

The term “pharmaceutically acceptable derivative” in the present context includes pharmaceutically acceptable salts, which indicate a salt which is not harmful to the patient. Such salts include pharmaceutically acceptable basic or acid addition salts as well as pharmaceutically acceptable metal salts, ammonium salts and alkylated ammonium salts. A pharmaceutically acceptable derivative further includes pharmaceutically acceptable esters, prodrugs, or other precursors of a compound which may be biologically metabolized into the active compound, or crystal forms of a compound.

The pharmaceutical composition or pharmaceutically acceptable composition may be specifically formulated for administration by any suitable route, such as an enteral route, the oral, rectal, nasal, pulmonary, buccal, sublingual, transdermal, intracisternal, intraperitoneal, and parenteral (including subcutaneous, intramuscular, intrathecal, intravenous and intradermal) route.

Pharmaceutical compositions for oral administration include solid dosage forms such as hard or soft capsules, tablets, troches, dragees, pills, lozenges, powders and granules. Where appropriate, they can be prepared with coatings such as enteric coatings, or they can be formulated so as to provide controlled release of the active ingredient, such as sustained or prolonged release, according to methods well known in the art. In the same solid dosage form two active ingredients may be combined so as to provide controlled release of one active ingredient and immediate release of another active ingredient.

Liquid dosage forms for oral administration include solutions, emulsions, aqueous or oily suspensions, syrups and elixirs.

Pharmaceutical compositions for parenteral administration include sterile aqueous and non-aqueous injectable solutions, dispersions, suspensions or emulsions, as well as sterile powders to be reconstituted in sterile injectable solutions or dispersions prior to use. Depot injectable formulations are also regarded as being within the scope of the present disclosure. Other suitable administration forms include suppositories, sprays, ointments, cremes/lotions, gels, inhalants, dermal patches, implants, etc.

In one embodiment of the present disclosure, a polypeptide or polypeptide analogue as disclosed herein is generally utilized as a free substance or as a pharmaceutically derivative such as a pharmaceutically acceptable ester or such as a salt thereof. Examples of the latter are: an acid addition salt of a compound having a free base functionality, and a base addition salt of a compound having a free acid functionality. The term “pharmaceutically acceptable salt” refers to a non-toxic salt of a polypeptide or polypeptide analogue for use according to the present disclosure, which salts are generally prepared by reacting a free base with a suitable organic or inorganic acid, or by reacting an acid with a suitable organic or inorganic base. When a polypeptide or polypeptide analogue according to the present disclosure contains a free base functionality, such salts are prepared in a conventional manner by treating a solution or suspension of the compound with a chemical equivalent of a pharmaceutically acceptable acid. When a polypeptide or polypeptide analogue according to the present disclosure contains a free acid functionality, such salts are prepared in a conventional manner by treating a solution or suspension of the compound with a chemical equivalent of a pharmaceutically acceptable base. Physiologically acceptable salts of a polypeptide or polypeptide analogue with a hydroxy group include the anionic form of the compound in combination with a suitable cation, such as sodium or ammonium ion. Other salts which are not pharmaceutically acceptable may be useful in the preparation of polypeptide or polypeptide analogues, and they form a further aspect of the disclosure. Pharmaceutically acceptable acid addition salts include, but are not limited to, hydrochloride, hydrobromide, hydroiodide, nitrate, sulfate, bisulfate, phosphate, acid phosphate, isonicotinate, acetate, trifluoroacetate, trichloroacetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts.

In one embodiment of the present disclosure, the polypeptides or polypeptide analogues disclosed herein are in crystalline forms, for example co-crystallized forms or hydrates of crystalline forms.

The term “prodrug” refers to peptides that are rapidly transformed in vivo to yield the parent compound of the above formulae, for example, by hydrolysis in blood or by metabolism in cells, such as for example the cells of the basal ganglia. A thorough discussion is provided in T. Higuchi and V Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are hereby incorporated by reference. Examples of prodrugs include pharmaceutically acceptable, non-toxic esters of the compounds of the present disclosure. Esters of the compounds of the present disclosure may be prepared according to conventional methods “March's Advanced Organic Chemistry, 5^{th} Edition”. M. B. Smith & J. March, John Wiley & Sons, 2001.

In one embodiment, for parenteral administration, solutions of polypeptides or polypeptide analogues according to the present disclosure in sterile aqueous solution, in aqueous propylene glycol or in sesame or peanut oil are employed. Aqueous solutions should be suitably buffered where appropriate, and the liquid diluent rendered isotonic with, e.g., sufficient saline or glucose. Aqueous solutions are particularly suitable for intravenous, intramuscular, subcutaneous and intraperitoneal administration. The sterile aqueous media to be employed are all readily available by standard techniques known to those skilled in the art.

Suitable pharmaceutical carriers include inert solid diluents or fillers, sterile aqueous solutions and various organic solvents. Examples of solid carriers are lactose, terra alba, sucrose, cyclodextrin, talc, gelatine, agar, pectin, acacia, magnesium stearate, stearic acid and lower alkyl ethers of cellulose. Examples of liquid carriers are syrup, peanut oil, olive oil, phospholipids, fatty acids, fatty acid amines, polyoxyethylene and water. Moreover, the carrier or diluent may include any sustained release material known in the art, such as glyceryl monostearate or glyceryl distearate, alone or mixed with a wax. The pharmaceutical compositions formed by combining the compounds according to the present disclosure and the pharmaceutically acceptable carriers are then readily administered in a variety of dosage forms suitable for the disclosed routes of administration. The formulations may conveniently be presented in unit dosage form by methods known in the art of pharmacy.

Formulations of the present disclosure suitable for oral administration may be presented as discrete units, such as capsules or tablets, which each contain a predetermined amount of the active ingredient, and which may include a suitable excipient. Furthermore, the orally available formulations may be in the form of a powder or granules, a solution or suspension in an aqueous or non-aqueous liquid, or an oil-in-water or water-in-oil liquid emulsion.

Compositions intended for oral use may be prepared according to any known method, and such compositions may contain one or more agents selected from the group consisting of sweetening agents, flavouring agents, colouring agents and preserving agents in order to provide pharmaceutically elegant and palatable preparations. Tablets may contain the active ingredient(s) in admixture with non-toxic pharmaceutically acceptable excipients which are suitable for the manufacture of tablets. These excipients may, for example, be: inert diluents, such as calcium carbonate, sodium carbonate, lactose, calcium phosphate or sodium phosphate; granulating and disintegrating agents, for example corn starch or alginic acid; binding agents, for example, starch, gelatine or acacia; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

Formulations for oral use may also be presented as hard gelatine capsules where the active ingredient is mixed with an inert solid diluent, for example, calcium carbonate, calcium phosphate or kaolin, or a soft gelatine capsules wherein the active ingredient is mixed with water or an oil medium, for example peanut oil, liquid paraffin, or olive oil. Aqueous suspensions may contain the compound for use according to the present disclosure in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide such as lecithin, or condensation products of an alkylene oxide with fatty acids, for example polyoxyethylene stearate, or condensation products of ethylene oxide with long chain aliphatic alcohols, for example, heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more colouring agents, one or more flavouring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as a liquid paraffin. The oily suspensions may contain a thickening agent, for example beeswax, hard paraffin or cetyl alcohol. Sweetening agents such as those set forth above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above. Additional excipients, for example, sweetening, flavouring, and colouring agents may also be present.

The pharmaceutical compositions comprising polypeptides or polypeptide analogues according to the present disclosure may also be in the form of oil-in-water emulsions. The oily phase may be a vegetable oil, for example, olive oil or arachis oil, or a mineral oil, for example a liquid paraffin, or a mixture thereof. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative and flavouring and colouring agent. The pharmaceutical compositions may be in the form of a sterile injectable aqueous or oleaginous suspension. This suspension may be formulated according to the known methods using suitable dispersing or wetting agents and suspending agents described above. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution, and isotonic sodium chloride solution. In addition, sterile, fixed oils are conveniently employed as solvent or suspending medium. For this purpose, any bland fixed oil may be employed using synthetic mono- or diglycerides. In addition, fatty acids such as oleic acid find use in the preparation of injectables.

The compositions may also be in the form of suppositories for rectal administration of the compounds of the disclosure. These compositions can be prepared by mixing the polypeptides or polypeptide analogues disclosed herein with a suitable non-irritating excipient which is solid at ordinary temperatures but liquid at the rectal temperature and will thus melt in the rectum to release the drug. Such materials include, for example, cocoa butter and polyethylene glycols.

Polypeptides and polypeptide analogues of the present disclosure may also be administered in the form of liposome delivery systems, such as small unilamellar vesicles, large unilamellar vesicles, and multilamellar vesicles. Liposomes may be formed from a variety of phospholipids, such as but not limited to cholesterol, stearylamine or phosphatidylcholines.

In addition, some polypeptides and polypeptide analogues of the present disclosure may form solvates with water or common organic solvents. Such solvates are also encompassed within the scope of the disclosure.

Thus, a further embodiment of the present disclosure provides a pharmaceutical composition comprising a polypeptide or polypeptide analogue as disclosed herein, or a pharmaceutically acceptable salt, solvate, or prodrug thereof, and one or more pharmaceutically acceptable carriers, excipients, or diluents.

### EXAMPLES

### Example 1

### Functional Assessment of Annexin A1 (AnxA1) N-Terminal Fragments: Monocyte

### Phagocytosis Method:

AnxA1 N-terminal fragments were assessed in whole blood phagocytosis assay. Whole blood from human subjects (45 μL) was incubated for 1 h with control peptide Anxa1 2-50 (100 nM) and test peptides Compound 1 (100 nM), Compound 2 (100 nM) and Compound 3 (30 nM), or vehicle in a 96-well plate. 50 μl pHrodo E. coli BioParticles Conjugate (5 μg/well) was added to the wells, and the plate was incubated at 37° C., 5% CO₂ for 30 min. 150 μl of 4% PFA was added, and the plate incubated at 4° C. for 30 min. 30 μL from each well of the 96-well plate was transferred into flow cytometry tubes with 500 μL RPMI 1640 and 1 μL of Vybrant DyeCycle dye. Tubes were incubated for 30 min at 37° C. and 5% CO₂, followed by addition of 2.5 mL of RPMI 1640 Medium in each tube.

Samples were acquired on the Attune NxT Flow Cytometer. Monocyte phagocytosis was determined as an increase in pHrodo Green fluorescence relative to control sample.

Control peptide:
Control peptide 1 (AnxA1 (2-50)):
(SEQ ID NO: 74)
AMVSEFLKQAWFIENEEQEYVQTVKSSKGGPGSAVSPYPTFNPSSDVAA
Test peptides:
Compound 1 (AnxA1 2-29 (V24L)):
(SEQ ID NO: 50)
AMVSEFLKQAWFIENEEQEYVQTLKSSK
Compound 2 (AnxA1 2-34 (V24L)):
(SEQ ID NO: 55)
AMVSEFLKQAWFIENEEQEYVQTLKSSKGGPGS
Compound 3 (AnxA1 2-39 (V24L)):
(SEQ ID NO: 60)
AMVSEFLKQAWFIENEEQEYVQTLKSSKGGPGSAVSPY

### Results:

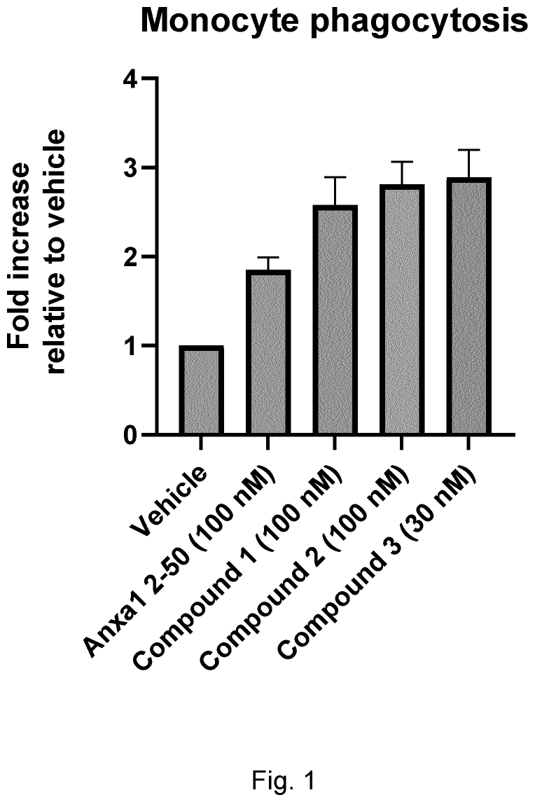

The results are shown in Table 1 below and FIG. 1. AnxA1 2-50 induces monocyte phagocytosis at a concentration of 100 nM. AnxA1 2-50 induces an almost 2-fold increase in monocyte phagocytosis compared to vehicle. Surprisingly, the shorter AnxA1 N-terminal fragments not only retained the ability to induce monocyte phagocytosis, but induce phagocytosis to a higher degree than AnxA1 2-50 (2.5 to 3-fold increase compared to vehicle).

TABLE 1
Fold increase relative to vehicle.

| | Fold increase relative to vehicle |
|---|---|
| Anxa1 2-50 | 1.9 |
| Compound 1 (AnxA1 2-29 (V24L)) | 2.6 |
| Compound 2 (AnxA1 2-34 (V24L)) | 2.8 |
| Compound 3 (AnxA1 2-39 (V24L)) | 2.9 |

### Example 2

### Functional Assessment of Annexin A1 (AnxA1) N-Terminal Fragments: Neutrophil Chemotaxis

### Method:

AnxA1 N-terminal fragments were assessed in polymorphonuclear leukocytes (PMNs) isolated from buffy coats of healthy volunteers. PMNs were resuspended in migration medium (RPMI1640 (ATCC, Cat. No. 30-2001)+0.05% BSA-FAF) to a concentration of 5.5×10⁶ cells/mL.

Receiver wells of a Transwell plate were filled with 200 μL of migration medium with 10 uM of compound 1, 2 or 3. Donor plate was inserted and 75 μL of cell suspension was added to upper wells (375,000 cells/well). Plates were incubated for 1 h (37° C., 5% CO₂, 95% humidity). The number of migrated cells was assessed by removing Transwell insert with cells from the plate in order to stop migration, followed by addition of 200 μL CellTiter Glo reagent to lower wells. Plates were incubated for 10 minutes in the dark (at room temperature), and 200 μL was transferred to white 96 well plates (Lumitrac 200). Luminescence was measured by use of EnVision 2104 (Perkin Elmer) (exposition time 0.1 s)

The chemotaxis response was estimated by calculating fold change compared to negative control (vehicle).

Test peptides:
Compound 1 (AnxA1 2-29 (V24L)):
(SEQ ID NO: 50)
AMVSEFLKQAWFIENEEQEYVQTLKSSK
Compound 2 (AnxA1 2-34 (V24L)):
(SEQ ID NO: 55)
AMVSEFLKQAWFIENEEQEYVQTLKSSKGGPGS
Compound 3 (AnxA1 2-39 (V24L)):
(SEQ ID NO: 60)
AMVSEFLKQAWFIENEEQEYVQTLKSSKGGPGSAVSPY

### Results:

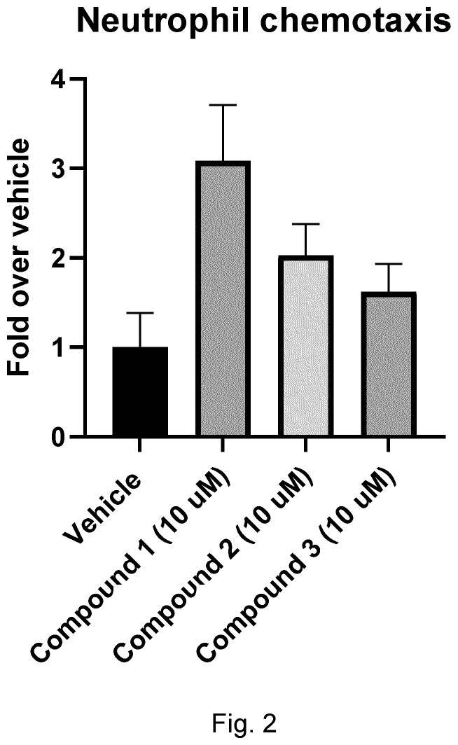

The results are shown in Table 2 below and FIG. 2. All three tested AnxA1 N-terminal fragments increase PMN chemotaxis compared to vehicle at a concentration of 10 μM. The effect was greatest with Compound 1 (AnxA1 2-29 (V24L)) which induces a 3-fold increase in chemotaxis compared to vehicle. Compound 2 (AnxA1 2-34 (V24L)) and Compound 3 (AnxA1 2-39 (V24L)) induce a 2-fold and 1.6-fold increase in PMN chemotaxis, respectively.

TABLE 2
Fold increase relative to vehicle

| | Fold increase relative to vehicle |
|---|---|
| Compound 1 (AnxA1 2-29 (V24L)) | 3.1 |
| Compound 2 (AnxA1 2-34 (V24L)) | 2.0 |
| Compound 3 (AnxA1 2-39 (V24L)) | 1.6 |

### Example 3

### Pharmacological Characterization of AnxA1 N-Terminal Fragments

### Method:

Recombinant cells co-expressing mitochondrial apoaequrin and recombinant human type 2 Formyl peptide receptor (FPR2) are grown for 18 hours prior to testing in media without antibiotics. The cells are then detached by gentle flushing with PBS-EDTA (5 mM EDTA), recovered by centrifugation and re-suspended in assay buffer (DHEM/HAM's F12 with HEPES and 0.1% protease free BSA). Following re-suspension in assay buffer the cells are then incubated for at least 4 hours at room temperature with coelenterazine h (from Molecular Probes) before dose response studies on receptor efficacy is conducted.

50 μl of cell suspension is then injected on 50 μl of the test or control compound (solubilized in PBS/0.5% BSA and finally diluted from a stock solution of 1 mM) at increasing concentrations in 96 wells plates and incubated for 30 min at room temperature. The resulting light emission is recorded using the Hamamatsu functional drug Screening system 6000 (FDSS6000). For standardization of emission of recorded light across plates and between experiments 100 μM digitonin or 20 μM ATP is added to some of the wells.

Agonist activity is expressed as % of the maximal activity obtained with an internal control compound. The test compounds are tested in a concentration range from 10-11 to 10-5 M. Data is presented as mean values. EC₅₀ (ie the concentration induced 50% of max response) is determined by best fit analyses after logarithmic transformation using the graph pad software (version 6.0).

Control peptides
Control peptide 1 (AnxA1 (2-26)):
(SEQ ID NO: 71)
Ac-Ala-Met-Val-Ser-Glu-Phe-Leu-Lys-Gln-Ala-Trp-
Phe-Ile-Glu-Asn-Glu-Glu-Gln-Glu-Tyr-Val-Gln-Thr-
Val-Lys-OH
Control peptide 2 (AnxA1 (2-12)):
(SEQ ID NO: 72)
Ac-Ala-Met-Val-Ser-Glu-Phe-Leu-Lys-Gln-Ala-Trp-NH₂
Control peptide 3 (W-peptide):
(SEQ ID NO: 73)
Trp-Lys-Tyr-Met-Val-Met
Test peptide analogues:
Analogue 1 (AnxA1 (2-27)):
(SEQ ID NO: 2)
Ac-Ala Met Val Ser Glu Phe Leu Lys Gln Ala Trp Phe
Ile Glu Asn Glu Glu Gln Glu Tyr Val Gln Thr Val
Lys Ser
Analogue 2 (AnxA1 (2-31)):
(SEQ ID NO: 6)
Ac-Ala Met Val Ser Glu Phe Leu Lys Gln Ala Trp Phe
Ile Glu Asn Glu Glu Gln Glu Tyr Val Gln Thr Val
Lys Ser Ser Lys Gly Gly
Analogue 3 (AnxA1 (2-35)):
(SEQ ID NO: 10)
Ac-Ala Met Val Ser Glu Phe Leu Lys Gln Ala Trp Phe
Ile Glu Asn Glu Glu Gln Glu Tyr Val Gln Thr Val
Lys Ser Ser Lys Gly Gly Pro Gly Ser Ala
Analogue 4 (AnxA1 (2-39)):
(SEQ ID NO: 14)
Ac-Ala Met Val Ser Glu Phe Leu Lys Gln Ala Trp Phe
Ile Glu Asn Glu Glu Gln Glu Tyr Val Gln Thr Val
Lys Ser Ser Lys Gly Gly Pro Gly Ser Ala Val Ser
Pro Tyr
Analogue 5 (AnxA1 (2-43)):
(SEQ ID NO: 18)
Ac-Ala Met Val Ser Glu Phe Leu Lys Gln Ala Trp Phe
Ile Glu Asn Glu Glu Gln Glu Tyr Val Gln Thr Val
Lys Ser Ser Lys Gly Gly Pro Gly Ser Ala Val Ser
Pro Tyr Pro Thr Phe Asn
Analogue 6 (AnxA1 (2-47)):
(SEQ ID NO: 21)
Ac-Ala Met Val Ser Glu Phe Leu Lys Gln Ala Trp Phe
Ile Glu Asn Glu Glu Gln Glu Tyr Val Gln Thr Val
Lys Ser Ser Lys Gly Gly Pro Gly Ser Ala Val Ser
Pro Tyr Pro Thr Phe Asn Pro Ser Ser Asp
Analogue 7 (AnxA1 (2-54)):
(SEQ ID NO: 24)
Ac-Ala Met Val Ser Glu Phe Leu Lys Gln Ala Trp Phe
Ile Glu Asn Glu Glu Gln Glu Tyr Val Gln Thr Val
Lys Ser Ser Lys Gly Gly Pro Gly Ser Ala Val Ser
Pro Tyr Pro Thr Phe Asn Pro Ser Ser Asp Val Ala
Ala Leu His Lys Ala

1-63. (canceled)

64. A polypeptide or polypeptide analogue comprising at least the sequence AMVSEFLKQAWFIENEEQEYVQTVKS (SEQ ID NO: 2), or a functional variant thereof having one, two, three, four or five amino acid substitutions,
wherein said polypeptide consists of a polypeptide selected from:
(SEQ ID NO: 2)
AMVSEFLKQAWFIENEEQEYVQTVKS,
(SEQ ID NO: 3)
AMVSEFLKQAWFIENEEQEYVQTVKSS,
(SEQ ID NO: 4)
AMVSEFLKQAWFIENEEQEYVQTVKSSK,
(SEQ ID NO: 5)
AMVSEFLKQAWFIENEEQEYVQTVKSSKG,
(SEQ ID NO: 6)
AMVSEFLKQAWFIENEEQEYVQTVKSSKGG,
(SEQ ID NO: 7)
AMVSEFLKQAWFIENEEQEYVQTVKSSKGGP,
(SEQ ID NO: 8)
AMVSEFLKQAWFIENEEQEYVQTVKSSKGGPG,
(SEQ ID NO: 9)
AMVSEFLKQAWFIENEEQEYVQTVKSSKGGPGS,
(SEQ ID NO: 10)
AMVSEFLKQAWFIENEEQEYVQTVKSSKGGPGSA,
(SEQ ID NO: 11)
AMVSEFLKQAWFIENEEQEYVQTVKSSKGGPGSAV,
(SEQ ID NO: 12)
AMVSEFLKQAWFIENEEQEYVQTVKSSKGGPGSAVS,
(SEQ ID NO: 13)
AMVSEFLKQAWFIENEEQEYVQTVKSSKGGPGSAVSP,
(SEQ ID NO: 14)
AMVSEFLKQAWFIENEEQEYVQTVKSSKGGPGSAVSPY,
(SEQ ID NO: 15)
AMVSEFLKQAWFIENEEQEYVQTVKSSKGGPGSAVSPYP,
(SEQ ID NO: 16)
AMVSEFLKQAWFIENEEQEYVQTVKSSKGGPGSAVSPYPT,
(SEQ ID NO: 17)
AMVSEFLKQAWFIENEEQEYVQTVKSSKGGPGSAVSPYPTF,
(SEQ ID NO: 18)
AMVSEFLKQAWFIENEEQEYVQTVKSSKGGPGSAVSPYPTFN,
(SEQ ID NO: 19)
AMVSEFLKQAWFIENEEQEYVQTVKSSKGGPGSAVSPYPTFNP,
(SEQ ID NO: 20)
AMVSEFLKQAWFIENEEQEYVQTVKSSKGGPGSAVSPYPTFNPS,
(SEQ ID NO: 21)
AMVSEFLKQAWFIENEEQEYVQTVKSSKGGPGSAVSPYPTFNPSSD,
(SEQ ID NO: 22)
AMVSEFLKQAWFIENEEQEYVQTVKSSKGGPGSAVSPYPTFNPSSDVAAL
H,
(SEQ ID NO: 23)
AMVSEFLKQAWFIENEEQEYVQTVKSSKGGPGSAVSPYPTFNPSSDVAAL
HK,
(SEQ ID NO: 24)
AMVSEFLKQAWFIENEEQEYVQTVKSSKGGPGSAVSPYPTFNPSSDVAAL
HKA,
and
a functional variant of any one of SEQ ID NO:2 to SEQ ID NO:24 having one, two, three, four or five amino acid substitutions.

65. The polypeptide or polypeptide analogue of claim 64, wherein said functional variant is a polypeptide selected from any one of SEQ ID NO:2 to SEQ ID NO:24 having one amino acid substitution.

66. The polypeptide or polypeptide analogue of claim 64, wherein said functional variant is a polypeptide selected from any one of SEQ ID NO:2 to SEQ ID NO:24 having two amino acid substitutions.

67. The polypeptide or polypeptide analogue of claim 64, wherein said functional variant is a polypeptide selected from any one of SEQ ID NO:2 to SEQ ID NO:24 having three amino acid substitutions.

68. The polypeptide or polypeptide analogue of claim 64, wherein the valine residue at position 24 of any one of SEQ ID NO: 2 to SEQ ID NO: 24 is substituted with an amino acid residue independently selected from leucine, aspartic acid, methionine, glutamic acid, isoleucine, arginine and lysine.

69. The polypeptide or polypeptide analogue of claim 64, wherein the valine residue at position 24 of any one of SEQ ID NO: 2 to SEQ ID NO: 24 is substituted with leucine.

70. The polypeptide or polypeptide analogue of claim 64, selected from:
(SEQ ID NO: 25)
AMVSEFLKQX₁WFIENEEQEYX₂QTX₃KS,
(SEQ ID NO: 26)
AMVSEFLKQX₁WFIENEEQEYX₂QTX₃KSS,
(SEQ ID NO: 27)
AMVSEFLKQX₁WFIENEEQEYX₂QTX₃KSSK,
(SEQ ID NO: 28)
AMVSEFLKQX₁WFIENEEQEYX₂QTX₃KSSKG,
(SEQ ID NO: 29)
AMVSEFLKQX₁WFIENEEQEYX₂QTX₃KSSKGG,
(SEQ ID NO: 30)
AMVSEFLKQX₁WFIENEEQEYX₂QTX₃KSSKGGP,
(SEQ ID NO: 31)
AMVSEFLKQX₁WFIENEEQEYX₂QTX₃KSSKGGPG,
(SEQ ID NO: 32)
AMVSEFLKQX₁WFIENEEQEYX₂QTX₃KSSKGGPGS,
(SEQ ID NO: 33)
AMVSEFLKQX₁WFIENEEQEYX₂QTX₃KSSKGGPGSA,
(SEQ ID NO: 34)
AMVSEFLKQX₁WFIENEEQEYX₂QTX₃KSSKGGPGSAX₄,
(SEQ ID NO: 35)
AMVSEFLKQX₁WFIENEEQEYX₂QTX₃KSSKGGPGSAX₄S,
(SEQ ID NO: 36)
AMVSEFLKQX₁WFIENEEQEYX₂QTX₃KSSKGGPGSAX₄SP,
(SEQ ID NO: 37)
AMVSEFLKQX₁WFIENEEQEYX₂QTX₃KSSKGGPGSAX₄SPY,
(SEQ ID NO: 38)
AMVSEFLKQX₁WFIENEEQEYX₂QTX₃KSSKGGPGSAX₄SPYP,
(SEQ ID NO: 39)
AMVSEFLKQX₁WFIENEEQEYX₂QTX₃KSSKGGPGSAX₄SPYPT,
(SEQ ID NO: 40)
AMVSEFLKQX₁WFIENEEQEYX₂QTX₃KSSKGGPGSAX₄SPYPTF,
(SEQ ID NO: 41)
AMVSEFLKQX₁WFIENEEQEYX₂QTX₃KSSKGGPGSAX₄SPYPTFN,
(SEQ ID NO: 42)
AMVSEFLKQX₁WFIENEEQEYX₂QTX₃KSSKGGPGSAX₄SPYPTFNP,
(SEQ ID NO: 43)
AMVSEFLKQX₁WFIENEEQEYX₂QTX₃KSSKGGPGSAX₄SPYPTFNPS,
(SEQ ID NO: 44)
AMVSEFLKQX₁WFIENEEQEYX₂QTX₃KSSKGGPGSAX₄SPYPTFNPSS
D,
(SEQ ID NO: 45)
AMVSEFLKQX₁WFIENEEQEYX₂QTX₃KSSKGGPGSAX₄SPYPTFNPSSD
VAAX₅H,
(SEQ ID NO: 46)
AMVSEFLKQX₁WFIENEEQEYX₂QTX₃KSSKGGPGSAX₄SPYPTFNPSSD
VAAX₅HK,
and
(SEQ ID NO: 47)
AMVSEFLKQX₁WFIENEEQEYX₂QTX₃KSSKGGPGSAX₄SPYPTFNPSSD
VAAX₅HKA,
wherein X₁ is selected from alanine, leucine, aspartic acid, methionine, glutamic acid, isoleucine and arginine,
wherein X₂ is selected from valine, leucine, aspartic acid, methionine, glutamic acid, isoleucine and lysine,
wherein X₃ is selected from valine, glycine, alanine, serine, threonine, cysteine, leucine, isoleucine, methionine, proline, phenylalanine, tyrosine, tryptophan, aspartic acid, glutamic acid, asparagine, glutamine, histidine, lysine and arginine,
wherein X₄ is selected from valine, glycine, alanine, serine, threonine, cysteine, leucine, isoleucine, methionine, proline, phenylalanine, tyrosine, tryptophan, aspartic acid, glutamic acid, asparagine, glutamine, histidine, lysine and arginine, and
wherein X₅ is selected from leucine, glycine, alanine, serine, threonine, cysteine, valine, isoleucine, methionine, proline, phenylalanine, tyrosine, tryptophan, aspartic acid, glutamic acid, asparagine, glutamine, histidine, lysine and arginine.

71. The polypeptide or polypeptide analogue of claim 64, selected from:
SEQ ID NO: 48)
AMVSEFLKQAWFIENEEQEYVQTLKS,
(SEQ ID NO: 49)
AMVSEFLKQAWFIENEEQEYVQTLKSS,
(SEQ ID NO: 50)
AMVSEFLKQAWFIENEEQEYVQTLKSSK,
(SEQ ID NO: 51)
AMVSEFLKQAWFIENEEQEYVQTLKSSKG,
(SEQ ID NO: 52)
AMVSEFLKQAWFIENEEQEYVQTLKSSKGG,
(SEQ ID NO: 53)
AMVSEFLKQAWFIENEEQEYVQTLKSSKGGP,
(SEQ ID NO: 54)
AMVSEFLKQAWFIENEEQEYVQTLKSSKGGPG,
(SEQ ID NO: 55)
AMVSEFLKQAWFIENEEQEYVQTLKSSKGGPGS,
(SEQ ID NO: 56)
AMVSEFLKQAWFIENEEQEYVQTLKSSKGGPGSA,
(SEQ ID NO: 57)
AMVSEFLKQAWFIENEEQEYVQTLKSSKGGPGSAV,
(SEQ ID NO: 58)
AMVSEFLKQAWFIENEEQEYVQTLKSSKGGPGSAVS,
(SEQ ID NO: 59)
AMVSEFLKQAWFIENEEQEYVQTLKSSKGGPGSAVSP,
(SEQ ID NO: 60)
AMVSEFLKQAWFIENEEQEYVQTLKSSKGGPGSAVSPY,
(SEQ ID NO: 61)
AMVSEFLKQAWFIENEEQEYVQTLKSSKGGPGSAVSPYP,
(SEQ ID NO: 62)
AMVSEFLKQAWFIENEEQEYVQTLKSSKGGPGSAVSPYPT,
(SEQ ID NO: 63)
AMVSEFLKQAWFIENEEQEYVQTLKSSKGGPGSAVSPYPTF,
(SEQ ID NO: 64)
AMVSEFLKQAWFIENEEQEYVQTLKSSKGGPGSAVSPYPTFN,
(SEQ ID NO: 65)
AMVSEFLKQAWFIENEEQEYVQTLKSSKGGPGSAVSPYPTFNP,
(SEQ ID NO: 66)
AMVSEFLKQAWFIENEEQEYVQTLKSSKGGPGSAVSPYPTFNPS,
(SEQ ID NO: 67)
AMVSEFLKQAWFIENEEQEYVQTLKSSKGGPGSAVSPYPTFNPSSD,
(SEQ ID NO: 68)
AMVSEFLKQAWFIENEEQEYVQTLKSSKGGPGSAVSPYPTFNPSSDVAAL
H,
(SEQ ID NO: 69)
AMVSEFLKQAWFIENEEQEYVQTLKSSKGGPGSAVSPYPTFNPSSDVAAL
HK,
and
(SEQ ID NO: 70)
AMVSEFLKQAWFIENEEQEYVQTLKSSKGGPGSAVSPYPTFNPSSDVAAL
HKA,
and
a functional variant of any one of SEQ ID NO:48 to SEQ ID NO:70 having 1, 2, 3 or 4 amino acid substitutions.

72. The polypeptide or polypeptide analogue of claim 64, selected from:
(SEQ ID NO: 50)
AMVSEFLKQAWFIENEEQEYVQTLKSSK,
(SEQ ID NO: 51)
AMVSEFLKQAWFIENEEQEYVQTLKSSKG,
(SEQ ID NO: 52)
AMVSEFLKQAWFIENEEQEYVQTLKSSKGG,
(SEQ ID NO: 53)
AMVSEFLKQAWFIENEEQEYVQTLKSSKGGP,
(SEQ ID NO: 54)
AMVSEFLKQAWFIENEEQEYVQTLKSSKGGPG,
(SEQ ID NO: 55)
AMVSEFLKQAWFIENEEQEYVQTLKSSKGGPGS,
(SEQ ID NO: 56)
AMVSEFLKQAWFIENEEQEYVQTLKSSKGGPGSA,
(SEQ ID NO: 57)
AMVSEFLKQAWFIENEEQEYVQTLKSSKGGPGSAV,
(SEQ ID NO: 58)
AMVSEFLKQAWFIENEEQEYVQTLKSSKGGPGSAVS,
(SEQ ID NO: 59)
AMVSEFLKQAWFIENEEQEYVQTLKSSKGGPGSAVSP,
and
(SEQ ID NO: 60)
AMVSEFLKQAWFIENEEQEYVQTLKSSKGGPGSAVSPY,
and
a functional variant of any one of SEQ ID NO:50 to SEQ ID NO:60 having 1, 2, 3, or 4 amino acid substitutions.

73. The polypeptide or polypeptide analogue of claim 64, selected from:
(SEQ ID NO: 50)
AMVSEFLKQAWFIENEEQEYVQTLKSSK,
(SEQ ID NO: 55)
AMVSEFLKQAWFIENEEQEYVQTLKSSKGGPGS,
and
(SEQ ID NO: 60)
AMVSEFLKQAWFIENEEQEYVQTLKSSKGGPGSAVSPY,
and
a functional variant of any one of SEQ ID NO:50, SEQ ID NO:55 and SEQ ID NO:60 having 1, 2, 3 or 4 amino acid substitutions.

74. The polypeptide or polypeptide analogue of claim 71, wherein said functional variant is a polypeptide selected from any one of SEQ ID NO:48 to SEQ ID NO:70 having one, two or three amino acid substitutions.

75. The polypeptide or polypeptide analogue of claim 64, wherein the polypeptide or polypeptide analogue is acetylated and/or wherein the C-terminal amino acid residue is amidated (—NH₂).

76. The polypeptide or polypeptide analogue of claim 64, wherein said polypeptide or functional variant thereof
a. binds to one or more of the formyl peptide receptors, including Formyl Peptide Receptor 1 (FPR1), Formyl Peptide Receptor 2 (FPR2) and Formyl Peptide Receptor 3 (FPR3), and/or
b. activates and/or stimulates one or more of the formyl peptide receptors, including Formyl Peptide Receptor 1 (FPR1), Formyl Peptide Receptor 2 (FPR2) and Formyl Peptide Receptor 3 (FPR3), and/or
c. is a ligand and/or agonist of one or more of the formyl peptide receptors, including Formyl Peptide Receptor 1 (FPR1), Formyl Peptide Receptor 2 (FPR2) and Formyl Peptide Receptor 3 (FPR3), and/or
d. binds, activates and/or is an agonist for FPR2, and/or
e. activates immune cells, and/or
f. activates leukocytes, and/or
g. activates phagocytic leukocytes or polymorphonuclear leukocytes (PMNs), and/or
h. activates neutrophils and/or monocytes, and/or
i. activates leukocytes' effector functions, and/or
j. Induces phagocytosis in leukocytes, and/or
k. induces chemotaxis in leukocytes.

77. A polypeptide conjugate comprising a polypeptide or polypeptide analogue of claim 64 and one or more branched amino acid probes,
wherein said branched amino acid probe comprises a first amino alkyl amino acid residue,
wherein the side chain of one or more of said first, second and/or third amino alkyl amino acid residues are each modified by attaching to the side chain amino group a molecule independently selected AAAq-AAA; (aa3)p-AAAq; AAAq-(aa3)p; [(aa3)-AAA]p and [AAA-(aa3)]p;
wherein q is a number selected from 0, 1, 2 and 3; p is a number selected from 1, 2 and 3; AAA is an amino alkyl amino acid residue; and (aa₃) is an amino acid residue independently selected from Arg, His, Gly and Ala,
wherein said first amino alkyl amino acid residue is covalently linked to the N-terminus of said polypeptide, covalently linked to the C-terminus of said polypeptide, and/or covalently linked to the side chain amino group of an amino alkyl amino acid residue within said polypeptide,
with the proviso that said branched amino acid probe consists of 2 to 9 amino acid residues.

78. A pharmaceutical composition comprising the polypeptide or polypeptide analogue of claim 64.

79. A method for treatment of an ischemic condition and/or an inflammatory condition comprising administering an effective amount of the polypeptide or polypeptide analogue of claim 64 to an individual in need thereof.

80. The method according to claim 79, wherein said ischemic condition is secondary ischemia.

81. The method according to claim 79, wherein said ischemic condition is due to stroke, injury, septic shock, systemic hypotension, cardiac arrest due to heart attack, cardiac arrhythmia, atheromatous disease with thrombosis, embolism from the heart or from blood vessel from any organ, vasospasm, aortic aneurysm or aneurisms in other organs, coronary stenosis, myocardial infarction, angina pectoris, pericarditis, myocarditis, myxodemia, or endocarditis.

82. The method according to claim 79, wherein said ischemic condition and/or inflammatory condition is associated with surgery or is associated with organ transplantation.

83. The method according to claim 79, wherein said ischemic condition and/or inflammatory condition is reperfusion injury.


SEQUENCE LISTING (76 sequences)

SEQ ID NO: 1 - protein, 346 aa, Homo sapiens
MAMVSEFLKQAWFIENEEQEYVQTVKSSKGGPGSAVSPYPTFNPSSDVAALHKAIMVKGVDEATIIDILTKRNNAQRQQIKAAYLQETGKPLDETLKKALTGHLEEVVLALLKTPAQFDADELRAAMKGLGTDEDTLIEILASRTNKEIRDINRVYREELKRDLAKDITSDTSGDFRNALLSLAKGDRSEDFGVNEDLADSDARALYEAGERRKGTDVNVFNTILTTRSYPQLRRVFQKYTKYSKHDMNKVLDLELKGDIEKCLTAIVKCATSKPAFFAEKLHQAMKGVGTRHKALIRIMVSRSEIDMNDIKAFYQKMYGISLCQAILDETKGDYEKILVALCGGN

SEQ ID NO: 2 - protein, 26 aa, Homo sapiens
AMVSEFLKQAWFIENEEQEYVQTVKS

SEQ ID NO: 3 - protein, 27 aa, Homo sapiens
AMVSEFLKQAWFIENEEQEYVQTVKSS

SEQ ID NO: 4 - protein, 28 aa, Homo sapiens
AMVSEFLKQAWFIENEEQEYVQTVKSSK

SEQ ID NO: 5 - protein, 29 aa, Homo sapiens
AMVSEFLKQAWFIENEEQEYVQTVKSSKG

SEQ ID NO: 6 - protein, 30 aa, Homo sapiens
AMVSEFLKQAWFIENEEQEYVQTVKSSKGG

SEQ ID NO: 7 - protein, 31 aa, Homo sapiens
AMVSEFLKQAWFIENEEQEYVQTVKSSKGGP

SEQ ID NO: 8 - protein, 32 aa, Homo sapiens
AMVSEFLKQAWFIENEEQEYVQTVKSSKGGPG

SEQ ID NO: 9 - protein, 33 aa, Homo sapiens
AMVSEFLKQAWFIENEEQEYVQTVKSSKGGPGS

SEQ ID NO: 10 - protein, 34 aa, Homo sapiens
AMVSEFLKQAWFIENEEQEYVQTVKSSKGGPGSA

SEQ ID NO: 11 - protein, 35 aa, Homo sapiens
AMVSEFLKQAWFIENEEQEYVQTVKSSKGGPGSAV

SEQ ID NO: 12 - protein, 36 aa, Homo sapiens
AMVSEFLKQAWFIENEEQEYVQTVKSSKGGPGSAVS

SEQ ID NO: 13 - protein, 37 aa, Homo sapiens
AMVSEFLKQAWFIENEEQEYVQTVKSSKGGPGSAVSP

SEQ ID NO: 14 - protein, 38 aa, Homo sapiens
AMVSEFLKQAWFIENEEQEYVQTVKSSKGGPGSAVSPY

SEQ ID NO: 15 - protein, 39 aa, Homo sapiens
AMVSEFLKQAWFIENEEQEYVQTVKSSKGGPGSAVSPYP

SEQ ID NO: 16 - protein, 40 aa, Homo sapiens
AMVSEFLKQAWFIENEEQEYVQTVKSSKGGPGSAVSPYPT

SEQ ID NO: 17 - protein, 41 aa, Homo sapiens
AMVSEFLKQAWFIENEEQEYVQTVKSSKGGPGSAVSPYPTF

SEQ ID NO: 18 - protein, 42 aa, Homo sapiens
AMVSEFLKQAWFIENEEQEYVQTVKSSKGGPGSAVSPYPTFN

SEQ ID NO: 19 - protein, 43 aa, Homo sapiens
AMVSEFLKQAWFIENEEQEYVQTVKSSKGGPGSAVSPYPTFNP

SEQ ID NO: 20 - protein, 44 aa, Homo sapiens
AMVSEFLKQAWFIENEEQEYVQTVKSSKGGPGSAVSPYPTFNPS

SEQ ID NO: 21 - protein, 46 aa, Homo sapiens
AMVSEFLKQAWFIENEEQEYVQTVKSSKGGPGSAVSPYPTFNPSSD

SEQ ID NO: 22 - protein, 51 aa, Homo sapiens
AMVSEFLKQAWFIENEEQEYVQTVKSSKGGPGSAVSPYPTFNPSSDVAALH

SEQ ID NO: 23 - protein, 52 aa, Homo sapiens
AMVSEFLKQAWFIENEEQEYVQTVKSSKGGPGSAVSPYPTFNPSSDVAALHK

SEQ ID NO: 24 - protein, 53 aa, Homo sapiens
AMVSEFLKQAWFIENEEQEYVQTVKSSKGGPGSAVSPYPTFNPSSDVAALHKA

SEQ ID NO: 25 - protein, 26 aa, synthetic construct
  REGION 1..26: note = AnxA1 fragment variant
  SITE 10: note = MISC_FEATURE - Xaa is Ala, Leu, Asp, Met, Glu, Ile or Arg
  SITE 21: note = MISC_FEATURE - Xaa is Val, Leu, Asp, Met, Glu, Ile, or Lys
  SITE 24: note = MISC_FEATURE - Xaa is Val, Gly, Ala, Ser, Thr, Cys, Leu, Ile, Met, Pro, Phe, Tyr, Trp, Asp, Glu, Asn, Gln, His, Lys, or Arg
AMVSEFLKQXWFIENEEQEYXQTXKS

SEQ ID NO: 26 - protein, 27 aa, synthetic construct
  REGION 1..27: note = AnxA1 fragment variant
  SITE 10: note = MISC_FEATURE - Xaa is Ala, Leu, Asp, Met, Glu, Ile or Arg
  SITE 21: note = MISC_FEATURE - Xaa is Val, Leu, Asp, Met, Glu, Ile, or Lys
  SITE 24: note = MISC_FEATURE - Xaa is Val, Gly, Ala, Ser, Thr, Cys, Leu, Ile, Met, Pro, Phe, Tyr, Trp, Asp, Glu, Asn, Gln, His, Lys, or Arg
AMVSEFLKQXWFIENEEQEYXQTXKSS

SEQ ID NO: 27 - protein, 28 aa, synthetic construct
  REGION 1..28: note = AnxA1 fragment variant
  SITE 10: note = MISC_FEATURE - Xaa is Ala, Leu, Asp, Met, Glu, Ile or Arg
  SITE 21: note = MISC_FEATURE - Xaa is Val, Leu, Asp, Met, Glu, Ile, or Lys
  SITE 24: note = MISC_FEATURE - Xaa is Val, Gly, Ala, Ser, Thr, Cys, Leu, Ile, Met, Pro, Phe, Tyr, Trp, Asp, Glu, Asn, Gln, His, Lys, or Arg
AMVSEFLKQXWFIENEEQEYXQTXKSSK

SEQ ID NO: 28 - protein, 29 aa, synthetic construct
  REGION 1..29: note = AnxA1 fragment variant
  SITE 10: note = MISC_FEATURE - Xaa is Ala, Leu, Asp, Met, Glu, Ile or Arg
  SITE 21: note = MISC_FEATURE - Xaa is Val, Leu, Asp, Met, Glu, Ile, or Lys
  SITE 24: note = MISC_FEATURE - Xaa is Val, Gly, Ala, Ser, Thr, Cys, Leu, Ile, Met, Pro, Phe, Tyr, Trp, Asp, Glu, Asn, Gln, His, Lys, or Arg
AMVSEFLKQXWFIENEEQEYXQTXKSSKG

SEQ ID NO: 29 - protein, 30 aa, synthetic construct
  REGION 1..30: note = AnxA1 fragment variant
  SITE 10: note = MISC_FEATURE - Xaa is Ala, Leu, Asp, Met, Glu, Ile or Arg
  SITE 21: note = MISC_FEATURE - Xaa is Val, Leu, Asp, Met, Glu, Ile, or Lys
  SITE 24: note = MISC_FEATURE - Xaa is Val, Gly, Ala, Ser, Thr, Cys, Leu, Ile, Met, Pro, Phe, Tyr, Trp, Asp, Glu, Asn, Gln, His, Lys, or Arg
AMVSEFLKQXWFIENEEQEYXQTXKSSKGG

SEQ ID NO: 30 - protein, 31 aa, synthetic construct
  REGION 1..31: note = AnxA1 fragment variant
  SITE 10: note = MISC_FEATURE - Xaa is Ala, Leu, Asp, Met, Glu, Ile or Arg
  SITE 21: note = MISC_FEATURE - Xaa is Val, Leu, Asp, Met, Glu, Ile, or Lys
  SITE 24: note = MISC_FEATURE - Xaa is Val, Gly, Ala, Ser, Thr, Cys, Leu, Ile, Met, Pro, Phe, Tyr, Trp, Asp, Glu, Asn, Gln, His, Lys, or Arg
AMVSEFLKQXWFIENEEQEYXQTXKSSKGGP

SEQ ID NO: 31 - protein, 32 aa, synthetic construct
  REGION 1..32: note = AnxA1 fragment variant
  SITE 10: note = MISC_FEATURE - Xaa is Ala, Leu, Asp, Met, Glu, Ile or Arg
  SITE 21: note = MISC_FEATURE - Xaa is Val, Leu, Asp, Met, Glu, Ile, or Lys
  SITE 24: note = MISC_FEATURE - Xaa is Val, Gly, Ala, Ser, Thr, Cys, Leu, Ile, Met, Pro, Phe, Tyr, Trp, Asp, Glu, Asn, Gln, His, Lys, or Arg
AMVSEFLKQXWFIENEEQEYXQTXKSSKGGPG

SEQ ID NO: 32 - protein, 33 aa, synthetic construct
  REGION 1..33: note = AnxA1 fragment variant
  SITE 10: note = MISC_FEATURE - Xaa is Ala, Leu, Asp, Met, Glu, Ile or Arg
  SITE 21: note = MISC_FEATURE - Xaa is Val, Leu, Asp, Met, Glu, Ile, or Lys
  SITE 24: note = MISC_FEATURE - Xaa is Val, Gly, Ala, Ser, Thr, Cys, Leu, Ile, Met, Pro, Phe, Tyr, Trp, Asp, Glu, Asn, Gln, His, Lys, or Arg
AMVSEFLKQXWFIENEEQEYXQTXKSSKGGPGS

SEQ ID NO: 33 - protein, 34 aa, synthetic construct
  REGION 1..34: note = AnxA1 fragment variant
  SITE 10: note = MISC_FEATURE - Xaa is Ala, Leu, Asp, Met, Glu, Ile or Arg
  SITE 21: note = MISC_FEATURE - Xaa is Val, Leu, Asp, Met, Glu, Ile, or Lys
  SITE 24: note = MISC_FEATURE - Xaa is Val, Gly, Ala, Ser, Thr, Cys, Leu, Ile, Met, Pro, Phe, Tyr, Trp, Asp, Glu, Asn, Gln, His, Lys, or Arg
AMVSEFLKQXWFIENEEQEYXQTXKSSKGGPGSA

SEQ ID NO: 34 - protein, 35 aa, synthetic construct
  REGION 1..35: note = AnxA1 fragment variant
  SITE 10: note = MISC_FEATURE - Xaa is Ala, Leu, Asp, Met, Glu, Ile or Arg
  SITE 21: note = MISC_FEATURE - Xaa is Val, Leu, Asp, Met, Glu, Ile, or Lys
  SITE 24: note = MISC_FEATURE - Xaa is Val, Gly, Ala, Ser, Thr, Cys, Leu, Ile, Met, Pro, Phe, Tyr, Trp, Asp, Glu, Asn, Gln, His, Lys, or Arg
  SITE 35: note = MISC_FEATURE - Xaa is Val, Gly, Ala, Ser, Thr, Cys, Leu, Ile, Met, Pro, Phe, Tyr, Trp, Asp, Glu, Asn, Gln, His, Lys, or Arg
AMVSEFLKQXWFIENEEQEYXQTXKSSKGGPGSAX

SEQ ID NO: 35 - protein, 36 aa, synthetic construct
  REGION 1..36: note = AnxA1 fragment variant
  SITE 10: note = MISC_FEATURE - Xaa is Ala, Leu, Asp, Met, Glu, Ile or Arg
  SITE 21: note = MISC_FEATURE - Xaa is Val, Leu, Asp, Met, Glu, Ile, or Lys
  SITE 24: note = MISC_FEATURE - Xaa is Val, Gly, Ala, Ser, Thr, Cys, Leu, Ile, Met, Pro, Phe, Tyr, Trp, Asp, Glu, Asn, Gln, His, Lys, or Arg
  SITE 35: note = MISC_FEATURE - Xaa is Val, Gly, Ala, Ser, Thr, Cys, Leu, Ile, Met, Pro, Phe, Tyr, Trp, Asp, Glu, Asn, Gln, His, Lys, or Arg
AMVSEFLKQXWFIENEEQEYXQTXKSSKGGPGSAXS

SEQ ID NO: 36 - protein, 37 aa, synthetic construct
  REGION 1..37: note = AnxA1 fragment variant
  SITE 10: note = MISC_FEATURE - Xaa is Ala, Leu, Asp, Met, Glu, Ile or Arg
  SITE 21: note = MISC_FEATURE - Xaa is Val, Leu, Asp, Met, Glu, Ile, or Lys
  SITE 24: note = MISC_FEATURE - Xaa is Val, Gly, Ala, Ser, Thr, Cys, Leu, Ile, Met, Pro, Phe, Tyr, Trp, Asp, Glu, Asn, Gln, His, Lys, or Arg
  SITE 35: note = MISC_FEATURE - Xaa is Val, Gly, Ala, Ser, Thr, Cys, Leu, Ile, Met, Pro, Phe, Tyr, Trp, Asp, Glu, Asn, Gln, His, Lys, or Arg
AMVSEFLKQXWFIENEEQEYXQTXKSSKGGPGSAXSP

SEQ ID NO: 37 - protein, 38 aa, synthetic construct
  REGION 1..38: note = AnxA1 fragment variant
  SITE 10: note = MISC_FEATURE - Xaa is Ala, Leu, Asp, Met, Glu, Ile or Arg
  SITE 21: note = MISC_FEATURE - Xaa is Val, Leu, Asp, Met, Glu, Ile, or Lys
  SITE 24: note = MISC_FEATURE - Xaa is Val, Gly, Ala, Ser, Thr, Cys, Leu, Ile, Met, Pro, Phe, Tyr, Trp, Asp, Glu, Asn, Gln, His, Lys, or Arg
  SITE 35: note = MISC_FEATURE - Xaa is Val, Gly, Ala, Ser, Thr, Cys, Leu, Ile, Met, Pro, Phe, Tyr, Trp, Asp, Glu, Asn, Gln, His, Lys, or Arg
AMVSEFLKQXWFIENEEQEYXQTXKSSKGGPGSAXSPY

SEQ ID NO: 38 - protein, 39 aa, synthetic construct
  REGION 1..39: note = AnxA1 fragment variant
  SITE 10: note = MISC_FEATURE - Xaa is Ala, Leu, Asp, Met, Glu, Ile or Arg
  SITE 21: note = MISC_FEATURE - Xaa is Val, Leu, Asp, Met, Glu, Ile, or Lys
  SITE 24: note = MISC_FEATURE - Xaa is Val, Gly, Ala, Ser, Thr, Cys, Leu, Ile, Met, Pro, Phe, Tyr, Trp, Asp, Glu, Asn, Gln, His, Lys, or Arg
  SITE 35: note = MISC_FEATURE - Xaa is Val, Gly, Ala, Ser, Thr, Cys, Leu, Ile, Met, Pro, Phe, Tyr, Trp, Asp, Glu, Asn, Gln, His, Lys, or Arg
AMVSEFLKQXWFIENEEQEYXQTXKSSKGGPGSAXSPYP

SEQ ID NO: 39 - protein, 40 aa, synthetic construct
  REGION 1..40: note = AnxA1 fragment variant
  SITE 10: note = MISC_FEATURE - Xaa is Ala, Leu, Asp, Met, Glu, Ile or Arg
  SITE 21: note = MISC_FEATURE - Xaa is Val, Leu, Asp, Met, Glu, Ile, or Lys
  SITE 24: note = MISC_FEATURE - Xaa is Val, Gly, Ala, Ser, Thr, Cys, Leu, Ile, Met, Pro, Phe, Tyr, Trp, Asp, Glu, Asn, Gln, His, Lys, or Arg
  SITE 35: note = MISC_FEATURE - Xaa is Val, Gly, Ala, Ser, Thr, Cys, Leu, Ile, Met, Pro, Phe, Tyr, Trp, Asp, Glu, Asn, Gln, His, Lys, or Arg
AMVSEFLKQXWFIENEEQEYXQTXKSSKGGPGSAXSPYPT

SEQ ID NO: 40 - protein, 41 aa, synthetic construct
  REGION 1..41: note = AnxA1 fragment variant
  SITE 10: note = MISC_FEATURE - Xaa is Ala, Leu, Asp, Met, Glu, Ile or Arg
  SITE 21: note = MISC_FEATURE - Xaa is Val, Leu, Asp, Met, Glu, Ile, or Lys
  SITE 24: note = MISC_FEATURE - Xaa is Val, Gly, Ala, Ser, Thr, Cys, Leu, Ile, Met, Pro, Phe, Tyr, Trp, Asp, Glu, Asn, Gln, His, Lys, or Arg
  SITE 35: note = MISC_FEATURE - Xaa is Val, Gly, Ala, Ser, Thr, Cys, Leu, Ile, Met, Pro, Phe, Tyr, Trp, Asp, Glu, Asn, Gln, His, Lys, or Arg
AMVSEFLKQXWFIENEEQEYXQTXKSSKGGPGSAXSPYPTF

SEQ ID NO: 41 - protein, 42 aa, synthetic construct
  REGION 1..42: note = AnxA1 fragment variant
  SITE 10: note = MISC_FEATURE - Xaa is Ala, Leu, Asp, Met, Glu, Ile or Arg
  SITE 21: note = MISC_FEATURE - Xaa is Val, Leu, Asp, Met, Glu, Ile, or Lys
  SITE 24: note = MISC_FEATURE - Xaa is Val, Gly, Ala, Ser, Thr, Cys, Leu, Ile, Met, Pro, Phe, Tyr, Trp, Asp, Glu, Asn, Gln, His, Lys, or Arg
  SITE 35: note = MISC_FEATURE - Xaa is Val, Gly, Ala, Ser, Thr, Cys, Leu, Ile, Met, Pro, Phe, Tyr, Trp, Asp, Glu, Asn, Gln, His, Lys, or Arg
AMVSEFLKQXWFIENEEQEYXQTXKSSKGGPGSAXSPYPTFN

SEQ ID NO: 42 - protein, 43 aa, synthetic construct
  REGION 1..43: note = AnxA1 fragment variant
  SITE 10: note = MISC_FEATURE - Xaa is Ala, Leu, Asp, Met, Glu, Ile or Arg
  SITE 21: note = MISC_FEATURE - Xaa is Val, Leu, Asp, Met, Glu, Ile, or Lys
  SITE 24: note = MISC_FEATURE - Xaa is Val, Gly, Ala, Ser, Thr, Cys, Leu, Ile, Met, Pro, Phe, Tyr, Trp, Asp, Glu, Asn, Gln, His, Lys, or Arg
  SITE 35: note = MISC_FEATURE - Xaa is Val, Gly, Ala, Ser, Thr, Cys, Leu, Ile, Met, Pro, Phe, Tyr, Trp, Asp, Glu, Asn, Gln, His, Lys, or Arg
AMVSEFLKQXWFIENEEQEYXQTXKSSKGGPGSAXSPYPTFNP

SEQ ID NO: 43 - protein, 44 aa, synthetic construct
  REGION 1..44: note = AnxA1 fragment variant
  SITE 10: note = MISC_FEATURE - Xaa is Ala, Leu, Asp, Met, Glu, Ile or Arg
  SITE 21: note = MISC_FEATURE - Xaa is Val, Leu, Asp, Met, Glu, Ile, or Lys
  SITE 24: note = MISC_FEATURE - Xaa is Val, Gly, Ala, Ser, Thr, Cys, Leu, Ile, Met, Pro, Phe, Tyr, Trp, Asp, Glu, Asn, Gln, His, Lys, or Arg
  SITE 35: note = MISC_FEATURE - Xaa is Val, Gly, Ala, Ser, Thr, Cys, Leu, Ile, Met, Pro, Phe, Tyr, Trp, Asp, Glu, Asn, Gln, His, Lys, or Arg
AMVSEFLKQXWFIENEEQEYXQTXKSSKGGPGSAXSPYPTFNPS

SEQ ID NO: 44 - protein, 46 aa, synthetic construct
  REGION 1..46: note = AnxA1 fragment variant
  SITE 10: note = MISC_FEATURE - Xaa is Ala, Leu, Asp, Met, Glu, Ile or Arg
  SITE 21: note = MISC_FEATURE - Xaa is Val, Leu, Asp, Met, Glu, Ile, or Lys
  SITE 24: note = MISC_FEATURE - Xaa is Val, Gly, Ala, Ser, Thr, Cys, Leu, Ile, Met, Pro, Phe, Tyr, Trp, Asp, Glu, Asn, Gln, His, Lys, or Arg
  SITE 35: note = MISC_FEATURE - Xaa is Val, Gly, Ala, Ser, Thr, Cys, Leu, Ile, Met, Pro, Phe, Tyr, Trp, Asp, Glu, Asn, Gln, His, Lys, or Arg
AMVSEFLKQXWFIENEEQEYXQTXKSSKGGPGSAXSPYPTFNPSSD

SEQ ID NO: 45 - protein, 51 aa, synthetic construct
  REGION 1..51: note = AnxA1 fragment variant
  SITE 10: note = MISC_FEATURE - Xaa is Ala, Leu, Asp, Met, Glu, Ile or Arg
  SITE 21: note = MISC_FEATURE - Xaa is Val, Leu, Asp, Met, Glu, Ile, or Lys
  SITE 24: note = MISC_FEATURE - Xaa is Val, Gly, Ala, Ser, Thr, Cys, Leu, Ile, Met, Pro, Phe, Tyr, Trp, Asp, Glu, Asn, Gln, His, Lys, or Arg
  SITE 35: note = MISC_FEATURE - Xaa is Val, Gly, Ala, Ser, Thr, Cys, Leu, Ile, Met, Pro, Phe, Tyr, Trp, Asp, Glu, Asn, Gln, His, Lys, or Arg
  SITE 50: note = MISC_FEATURE - Xaa is Leu, Gly, Ala, Ser, Thr, Cys, Val, Ile, Met, Pro, Phe, Tyr, Trp, Asp, Glu, Asn, Gln, His, Lys, or Arg
AMVSEFLKQXWFIENEEQEYXQTXKSSKGGPGSAXSPYPTFNPSSDVAAXH

SEQ ID NO: 46 - protein, 52 aa, synthetic construct
  REGION 1..52: note = AnxA1 fragment variant
  SITE 10: note = misc_feature - Xaa can be any naturally occurring amino acid
  SITE 21: note = misc_feature - Xaa can be any naturally occurring amino acid
  SITE 24: note = misc_feature - Xaa can be any naturally occurring amino acid
  SITE 35: note = misc_feature - Xaa can be any naturally occurring amino acid
  SITE 50: note = misc_feature - Xaa can be any naturally occurring amino acid
AMVSEFLKQXWFIENEEQEYXQTXKSSKGGPGSAXSPYPTFNPSSDVAAXHK

SEQ ID NO: 47 - protein, 53 aa, synthetic construct
  REGION 1..53: note = AnxA1 fragment variant
  SITE 10: note = MISC_FEATURE - Xaa is Ala, Leu, Asp, Met, Glu, Ile or Arg
  SITE 21: note = MISC_FEATURE - Xaa is Val, Leu, Asp, Met, Glu, Ile, or Lys
  SITE 24: note = MISC_FEATURE - Xaa is Val, Gly, Ala, Ser, Thr, Cys, Leu, Ile, Met, Pro, Phe, Tyr, Trp, Asp, Glu, Asn, Gln, His, Lys, or Arg
  SITE 35: note = MISC_FEATURE - Xaa is Val, Gly, Ala, Ser, Thr, Cys, Leu, Ile, Met, Pro, Phe, Tyr, Trp, Asp, Glu, Asn, Gln, His, Lys, or Arg
  SITE 50: note = MISC_FEATURE - Xaa is Leu, Gly, Ala, Ser, Thr, Cys, Val, Ile, Met, Pro, Phe, Tyr, Trp, Asp, Glu, Asn, Gln, His, Lys, or Arg
AMVSEFLKQXWFIENEEQEYXQTXKSSKGGPGSAXSPYPTFNPSSDVAAXHKA

SEQ ID NO: 48 - protein, 26 aa, synthetic construct
  REGION 1..26: note = Val24Leu variant of AnxA1 fragment
  REGION 1..26: note = MISC_FEATURE - Val24Leu variant of AnxA1 fragment
AMVSEFLKQAWFIENEEQEYVQTLKS

SEQ ID NO: 49 - protein, 27 aa, synthetic construct
  REGION 1..27: note = Val24Leu variant of AnxA1 fragment
  REGION 1..27: note = MISC_FEATURE - Val24Leu variant of AnxA1 fragment
AMVSEFLKQAWFIENEEQEYVQTLKSS

SEQ ID NO: 50 - protein, 28 aa, synthetic construct
  REGION 1..28: note = Val24Leu variant of AnxA1 fragment
  REGION 1..28: note = MISC_FEATURE - Val24Leu variant of AnxA1 fragment
AMVSEFLKQAWFIENEEQEYVQTLKSSK

SEQ ID NO: 51 - protein, 29 aa, synthetic construct
  REGION 1..29: note = Val24Leu variant of AnxA1 fragment
  REGION 1..29: note = MISC_FEATURE - Val24Leu variant of AnxA1 fragment
AMVSEFLKQAWFIENEEQEYVQTLKSSKG

SEQ ID NO: 52 - protein, 30 aa, synthetic construct
  REGION 1..30: note = Val24Leu variant of AnxA1 fragment
  REGION 1..30: note = MISC_FEATURE - Val24Leu variant of AnxA1 fragment
AMVSEFLKQAWFIENEEQEYVQTLKSSKGG

SEQ ID NO: 53 - protein, 31 aa, synthetic construct
  REGION 1..31: note = Val24Leu variant of AnxA1 fragment
  REGION 1..31: note = MISC_FEATURE - Val24Leu variant of AnxA1 fragment
AMVSEFLKQAWFIENEEQEYVQTLKSSKGGP

SEQ ID NO: 54 - protein, 32 aa, synthetic construct
  REGION 1..32: note = Val24Leu variant of AnxA1 fragment
  REGION 1..32: note = MISC_FEATURE - Val24Leu variant of AnxA1 fragment
AMVSEFLKQAWFIENEEQEYVQTLKSSKGGPG

SEQ ID NO: 55 - protein, 33 aa, synthetic construct
  REGION 1..33: note = Val24Leu variant of AnxA1 fragment
  REGION 1..33: note = MISC_FEATURE - Val24Leu variant of AnxA1 fragment
AMVSEFLKQAWFIENEEQEYVQTLKSSKGGPGS

SEQ ID NO: 56 - protein, 34 aa, synthetic construct
  REGION 1..34: note = Val24Leu variant of AnxA1 fragment
  REGION 1..34: note = MISC_FEATURE - Val24Leu variant of AnxA1 fragment
AMVSEFLKQAWFIENEEQEYVQTLKSSKGGPGSA

SEQ ID NO: 57 - protein, 35 aa, synthetic construct
  REGION 1..35: note = Val24Leu variant of AnxA1 fragment
  REGION 1..35: note = MISC_FEATURE - Val24Leu variant of AnxA1 fragment
AMVSEFLKQAWFIENEEQEYVQTLKSSKGGPGSAV

SEQ ID NO: 58 - protein, 36 aa, synthetic construct
  REGION 1..36: note = Val24Leu variant of AnxA1 fragment
  REGION 1..36: note = MISC_FEATURE - Val24Leu variant of AnxA1 fragment
AMVSEFLKQAWFIENEEQEYVQTLKSSKGGPGSAVS

SEQ ID NO: 59 - protein, 37 aa, synthetic construct
  REGION 1..37: note = Val24Leu variant of AnxA1 fragment
  REGION 1..37: note = MISC_FEATURE - Val24Leu variant of AnxA1 fragment
AMVSEFLKQAWFIENEEQEYVQTLKSSKGGPGSAVSP

SEQ ID NO: 60 - protein, 38 aa, synthetic construct
  REGION 1..38: note = Val24Leu variant of AnxA1 fragment
  REGION 1..38: note = MISC_FEATURE - Val24Leu variant of AnxA1 fragment
AMVSEFLKQAWFIENEEQEYVQTLKSSKGGPGSAVSPY

SEQ ID NO: 61 - protein, 39 aa, synthetic construct
  REGION 1..39: note = Val24Leu variant of AnxA1 fragment
  REGION 1..39: note = MISC_FEATURE - Val24Leu variant of AnxA1 fragment
AMVSEFLKQAWFIENEEQEYVQTLKSSKGGPGSAVSPYP

SEQ ID NO: 62 - protein, 40 aa, synthetic construct
  REGION 1..40: note = Val24Leu variant of AnxA1 fragment
  REGION 1..40: note = MISC_FEATURE - Val24Leu variant of AnxA1 fragment
AMVSEFLKQAWFIENEEQEYVQTLKSSKGGPGSAVSPYPT

SEQ ID NO: 63 - protein, 41 aa, synthetic construct
  REGION 1..41: note = Val24Leu variant of AnxA1 fragment
  REGION 1..41: note = MISC_FEATURE - Val24Leu variant of AnxA1 fragment
AMVSEFLKQAWFIENEEQEYVQTLKSSKGGPGSAVSPYPTF

SEQ ID NO: 64 - protein, 42 aa, synthetic construct
  REGION 1..42: note = Val24Leu variant of AnxA1 fragment
  REGION 1..42: note = MISC_FEATURE - Val24Leu variant of AnxA1 fragment
AMVSEFLKQAWFIENEEQEYVQTLKSSKGGPGSAVSPYPTFN

SEQ ID NO: 65 - protein, 43 aa, synthetic construct
  REGION 1..43: note = Val24Leu variant of AnxA1 fragment
  REGION 1..43: note = MISC_FEATURE - Val24Leu variant of AnxA1 fragment
AMVSEFLKQAWFIENEEQEYVQTLKSSKGGPGSAVSPYPTFNP

SEQ ID NO: 66 - protein, 44 aa, synthetic construct
  REGION 1..44: note = Val24Leu variant of AnxA1 fragment
  REGION 1..44: note = MISC_FEATURE - Val24Leu variant of AnxA1 fragment
AMVSEFLKQAWFIENEEQEYVQTLKSSKGGPGSAVSPYPTFNPS

SEQ ID NO: 67 - protein, 46 aa, synthetic construct
  REGION 1..46: note = Val24Leu variant of AnxA1 fragment
  REGION 1..46: note = MISC_FEATURE - Val24Leu variant of AnxA1 fragment
AMVSEFLKQAWFIENEEQEYVQTLKSSKGGPGSAVSPYPTFNPSSD

SEQ ID NO: 68 - protein, 51 aa, synthetic construct
  REGION 1..51: note = Val24Leu variant of AnxA1 fragment
  REGION 1..51: note = MISC_FEATURE - Val24Leu variant of AnxA1 fragment
AMVSEFLKQAWFIENEEQEYVQTLKSSKGGPGSAVSPYPTFNPSSDVAALH

SEQ ID NO: 69 - protein, 52 aa, synthetic construct
  REGION 1..52: note = Val24Leu variant of AnxA1 fragment
  REGION 1..52: note = MISC_FEATURE - Val24Leu variant of AnxA1 fragment
AMVSEFLKQAWFIENEEQEYVQTLKSSKGGPGSAVSPYPTFNPSSDVAALHK

SEQ ID NO: 70 - protein, 53 aa, synthetic construct
  REGION 1..53: note = Val24Leu variant of AnxA1 fragment
  REGION 1..53: note = MISC_FEATURE - Val24Leu variant of AnxA1 fragment
AMVSEFLKQAWFIENEEQEYVQTLKSSKGGPGSAVSPYPTFNPSSDVAALHKA

SEQ ID NO: 71 - protein, 25 aa, synthetic construct
  REGION 1..25: note = Control peptide 1
  REGION 1..25: note = MISC_FEATURE - AnxA1 fragment
  SITE 1: note = MISC_FEATURE - N-terminal acetylation
AMVSEFLKQAWFIENEEQEYVQTVK

SEQ ID NO: 72 - protein, 11 aa, synthetic construct
  REGION 1..11: note = Control peptide 2
  REGION 1..11: note = MISC_FEATURE - AnxA1 fragment
  SITE 1: note = MISC_FEATURE - N-terminal acetylation
  SITE 11: note = MISC_FEATURE - C-terminal amidation
AMVSEFLKQAW

SEQ ID NO: 73 - protein, 6 aa, synthetic construct
  REGION 1..6: note = Control peptide 3
  REGION 1..6: note = MISC_FEATURE - W-peptide
WKYMVM

SEQ ID NO: 74 - protein, 49 aa, synthetic construct
  REGION 1..49: note = Synthetic sequence having some homology with human annexin 1
AMVSEFLKQAWFIENEEQEYVQTVKSSKGGPGSAVSPYPTFNPSSDVAA

SEQ ID NO: 75 - protein, 4 aa, synthetic construct
LLLL

SEQ ID NO: 76 - protein, 4 aa, synthetic construct
  SITE 1: note = Acetylation of the N terminus
LLLL